(19) United States
(12) Patent Application Publication
(10) Pub. No.: US 2022/0347312 A1
(43) Pub. Date: Nov. 3, 2022

(54) Immunoconjugate Synthesis Method

(71) Applicant: Bolt Biotherapeutics, Inc., Redwood City, CA (US)

(72) Inventors: Puneet Anand, Redwood City, CA (US); David Dornan, Redwood City, CA (US); Romas Kudirka, Redwood City, CA (US); Richard P. Laura, Redwood City, CA (US); Arthur Lee, Redwood City, CA (US); Brian Safina, Redwood City, CA (US); Matthew Zhou, Redwood City, CA (US)

(73) Assignee: Bolt Biotherapeutics, Inc., Redwood City, CA (US)

(21) Appl. No.: 17/639,620

(22) PCT Filed: Sep. 4, 2020
(86) PCT No.: PCT/US2020/049401

§ 371 Date: Mar. 2, 2022

Related U.S. Application Data
(60) Provisional application No. 62/895,801, filed on Sep. 4, 2019.
(60) Provisional application No. 62/907,136, filed on Sep. 27, 2019.

(51) Int. Cl. A61K 47/68

(52) U.S. Cl. CPC A61K 47/6849; A61K 47/6845; A61K 47/6869; A61K 47/6865; A61K 47/6889

(57) ABSTRACT

The invention provides a method for producing an immunoconjugate, the method comprising combining one or more compounds of Formula I and an antibody construct of Formula II to provide the immunoconjugate of Formula III, wherein TA is a therapeutic agent, L is a linker, r is an integer from 1 to 50, Ar is an aromatic moiety comprising a substituent selected from PEG, —SO₂CX₃, —NR₃⁺, —NO₂, —SO₃R, —SO₂R, —CN, —CX₃, —PO₃R₂, —OPO₃R₂, and salts thereof, each R independently is H, CX₃, or C₁-C₄ alkyl, each X independently is hydrogen or a halogen, Y is CH₂, PEG, or a bond, n is an integer from 1 to 4, and PEG has the formula: —(CH₂CH₂O)m-(CH₂)ₚ—, where p is an integer from 1 to 5 and m is an integer from 2 to 50. The invention also provides an immunoconjugate and a composition of immunoconjugates formed from said method.

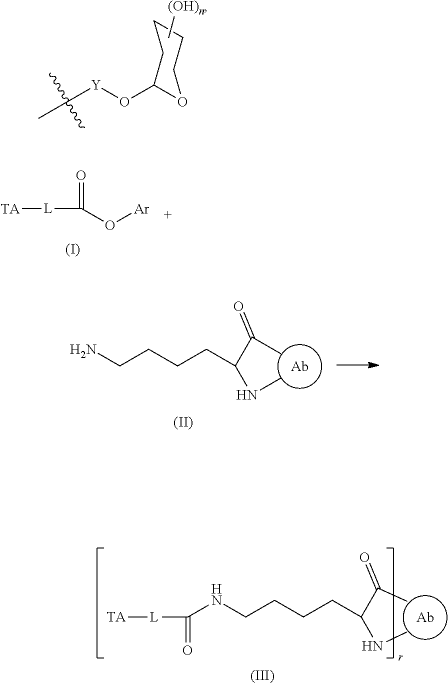

### CROSS-REFERENCE TO RELATED APPLICATIONS

This application claims the benefit of priority to U.S. Provisional Patent Application No. 62/895,801 filed Sep. 4, 2019, and U.S. Provisional Patent Application No. 62/907,136 filed Sep. 27, 2019, each of which is hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 102,888 Byte ASCII (Text) file named “750533_ST25.txt,” created on Sep. 3, 2020.

### BACKGROUND OF THE INVENTION

Unfortunately, the immune system is often not capable of controlling the growth and spread of cancer and other diseases and conditions. Antibodies and immune therapeutic agents have been shown to be effective treatments that assist the immune system in cancer and disease control. The simultaneous delivery of anti-tumor antibodies and therapeutic agents can be effective to treat tumors and to expand treatment options for cancer patients and other subjects. In addition, the simultaneous delivery of antibodies and therapeutic agents (i.e., immune agonists or immune antagonists) can be effective to treat diseases, conditions, and disorders, such as infections caused by viruses, bacteria, or parasites, and autoimmune diseases.

An effective way to simultaneously deliver antibodies and immune therapeutic agents is by conjugating the antibodies and therapeutic agents to form immunoconjugates. There are several methods that can be used to produce such immunoconjugates.

Examples of methods that can be used to produce immunoconjugates are well known in the art. Therapeutic agents can be covalently bonded to antibodies using various chemistries for protein modification, in which linking moieties result from the reaction of protein functional groups (i.e., amino acid side chains) with reagents having reactive linker groups. A wide variety of such reagents are known in the art. Examples of such reagents include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include but are not limited to the reagents described in Hermanson, Bioconjugate Techniques 2nd Edition, Academic Press, 2008.

One particular reference, namely, U.S. Pat. No. 8,741,291 (the '291 patent), discloses halogenated esters (e.g., TFP and PFP esters) for use in the conjugation of proteins or peptides to antibodies. The '291 patent describes that the conjugation method provides an added benefit of site selectivity, resulting in at least about 50% conjugation to the side chain of K⁸⁰ of the light chain kappa domain constant region (CLκ) (K¹⁸⁸ according to Kabat numbering). However, the method disclosed in the '291 patent may suffer from drawbacks (e.g., rate of reaction, selectivity, yield, etc.) associated with solubility, when extended beyond small peptides and proteins.

Thus, there remains a need for new methods for preparing immunoconjugates. The invention addresses this and other needs.

### BRIEF SUMMARY OF THE INVENTION

The invention provides a method for producing an immunoconjugate of a therapeutic agent and an antibody construct. The method comprises combining one or more compounds of Formula I:

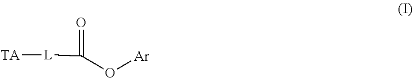

or salts thereof,
and an antibody construct of Formula II:

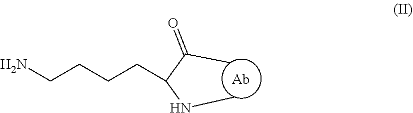

or salt thereof,

wherein Formula II is an antibody construct with residue

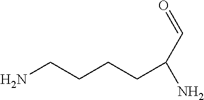

representing one or more lysine residues of the antibody construct (such that Ab represents the remainder of the antibody construct),

to provide the immunoconjugate of Formula III:

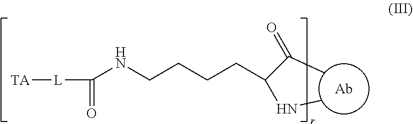

or salt thereof,
wherein TA is therapeutic agent, L is a linker, r is an integer from 1 to 50, Ar is an aromatic moiety comprising a substituent selected from PEG, —SO₂CX₃, —NR₃⁺, —NO₂, —SO₃R, —SO₂R, —CN, —CX₃, —PO₃R₂, —OPO₃R₂,

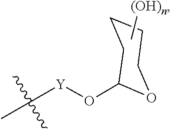

and salts thereof, each R independently is H, CX₃, or C₁-C₄ alkyl, each X independently is hydrogen or a halogen, Y is CH₂, PEG, or a bond, n is an integer from 1 to 4, and PEG has the formula: —(CH₂CH₂O)ₘ—(CH₂)ₚ—, where p is an integer from 1 to 5 and m is an integer from 2 to 50.

The invention also provides immunoconjugates prepared in accordance with the inventive production method, as well as compositions comprising such immunoconjugates.

The invention further provides a method for treating or preventing cancer comprising administering a therapeutically effective amount of an immunoconjugate or composition according to the invention to a subject in need thereof.

### BRIEF DESCRIPTION OF THE DRAWINGS

FIG. 1 is a graph of Time (hours) versus Area (%), which shows the hydrolytic stability of a therapeutic agent linker compound having a sulfo-tetrafluorophenyl ester (S-TFP Linker TA) and a therapeutic agent linker compound having a tetrflurophenyl ester (TFP Linker TA) in a DMA buffer solution, as described in Example 2.

FIG. 2 is a bar graph that shows the conjugation profile, as measured in percent conjugated to the light chain (LC), heavy chain (HC), and Lysine¹⁸⁸ of the light chain (LC K188), for immunoconjugates formed from conjugation with a tetrafluorophenyl ester (TFP), N-hydroxysuccinimide ester (NHS), sulfo tetrafluorophenyl ester (S-TFP), and sulfo dichlorophenyl ester (SDP), as described in Example 3.

### DETAILED DESCRIPTION OF THE INVENTION

### General

The invention provides a method for producing an immunoconjugate, the method comprising combining one or more compounds of Formula I:

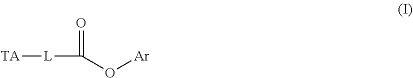

or salts thereof,
and an antibody construct of Formula II:

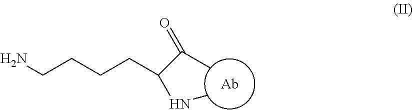

or salt thereof,

wherein Formula II is an antibody construct with residue

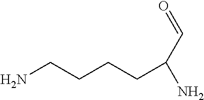

representing one or more lysine residues of the antibody construct (such that Ab represents the remainder of the antibody construct),

to provide the immunoconjugate of Formula III:

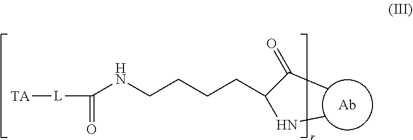

or salt thereof,
wherein

TA is a therapeutic agent,

L is a linker,

r is an integer from 1 to 50,

Ar is an aromatic moiety comprising a substituent selected from PEG, —SO₂CX₃, —NR₃⁺, —NO₂, —SO₃R, —SO₂R, —CN, —CX₃, —PO₃R₂, —OPO₃R₂,

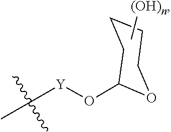

and salts thereof,

each R independently is H, CX₃, or C₁-C₄ alkyl,

each X independently is hydrogen or a halogen,

Y is CH₂, PEG, or a bond,

n is an integer from 1 to 4, and

PEG has the formula:

—(CH₂CH₂O)ₘ—(CH₂)ₚ—,

where p is an integer from 1 to 5 and m is an integer from 2 to 50.

The effectiveness of the method of making immunoconjugates described herein can be considered in terms of the efficiency and/or selectivity by which a therapeutic agent can be conjugated to an antibody construct via a linker. The ester moieties utilized to facilitate the method have particular electronic and/or steric properties that allow for a preferred combination of reactivity, solubility, and/or stability to provide a desired immunoconjugate or composition of immunoconjugates.

### Definitions

As used herein, the term “immunoconjugate” refers to an antibody construct, or antibody, that is covalently bonded to a non-naturally occurring chemical moiety as described herein. The terms “immunoconjugate” and are used interchangeably herein.

As used herein, the phrase “antibody construct” refers to polypeptide comprising an antigen binding domain and an Fc domain. An antibody construct can comprise or be an antibody.

As used herein, the phrase “antigen binding domain” refers to a protein, or a portion of a protein, that specifically binds a specified antigen (e.g., a paratope), for example, that portion of an antigen-binding protein that contains the amino acid residues that interact with an antigen and confer on the antigen-binding protein its specificity and affinity for the antigen.

As used herein, the phrase “Fc domain” refers to the fragment crystallizable region, or the tail region of an antibody. The Fc domain interacts with Fc receptors on cell surfaces.

As used herein, the phrase “targeting binding domain” refers to a protein, or a portion of a protein, that specifically binds a second antigen that is distinct from the antigen bound by the antigen binding domain of the immunoconjugates. The targeting binding domain can be conjugated to the antibody construct at a C-terminal end of the Fc domain.

As used herein, the term “antibody” refers to a polypeptide comprising an antigen binding region (including the complementarity determining region (CDRs)) from an immunoglobulin gene or fragments thereof that specifically binds and recognizes an antigen. The recognized immunoglobulin genes include the kappa, lambda, alpha, gamma, delta, epsilon, and mu constant region genes, as well as numerous immunoglobulin variable region genes. “Antibody” is used in the broadest sense and specifically encompasses monoclonal antibodies (including full length monoclonal antibodies), polyclonal antibodies, and multispecific antibodies (e.g., bispecific antibodies).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively. Light chains are classified as either kappa or lambda. Heavy chains are classified as gamma, mu, alpha, delta, or epsilon, which in turn define the immunoglobulin classes IgG, IgM, IgA, IgD, and IgE, respectively.

IgG antibodies are large molecules of about 150 kDa composed of four peptide chains. IgG antibodies contain two identical class γ heavy chains of about 50 kDa and two identical light chains of about 25 kDa, forming a tetrameric quaternary structure. The two heavy chains are linked to each other and to a light chain each by disulfide bonds. The resulting tetramer has two identical halves, which together form the Y-like shape. Each end of the fork contains an identical antigen binding site. There are four IgG subclasses (IgG1, 2, 3, and 4) in humans, named in order of their abundance in serum (IgG1 being the most abundant). Typically, the antigen-binding region of an antibody will be most critical in specificity and affinity of binding.

Dimeric IgA antibodies are about 320 kDa. IgA has two subclasses (IgA1 and IgA2) and can be produced as a monomeric as well as a dimeric form. The IgA dimeric form (secretory or sIgA) is the most abundant.

Antibodies can exist, for example, as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see, e.g., Fundamental Immunology (Paul, editor, 7th edition, 2012)). While various antibody fragments are defined in terms of the digestion of an intact antibody, such fragments may be synthesized de novo either chemically or by using recombinant DNA methodology. Thus, the term antibody, as used herein, also includes antibody fragments produced by the modification of whole antibodies, synthesized de novo using recombinant DNA methodologies (e.g., single chain Fv), or identified using phage display libraries (see, e.g., McCafferty et al., Nature, 348: 552-554 (1990)).

The term “antibody fragment” and all grammatical variants thereof as used herein are defined as a portion of an intact antibody comprising the antigen binding site or variable region of the intact antibody, wherein the portion is free of the constant heavy chain domains (i.e., CH₂, CH₃, and CH₄, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used herein, the term “biosimilar” in reference to a biological product means that the biological product is highly similar to the reference product notwithstanding minor differences in clinically inactive components, and there are no clinically meaningful differences between the biological product and the reference product in terms of the safety, purity, and potency of the product.

As used herein, the term “biobetter” refers to an approved antibody construct that is an improvement of a previously approved antibody construct (e.g., trastuzumab or pertuzumab). The biobetter can have one or more modifications (e.g., an altered glycan profile, or a unique epitope) over the previously approved antibody construct.

As used herein, the term “epitope” means any antigenic determinant on an antigen to which binds the antigen-binding site, also referred to as the paratope, of an antibody. Epitopic determinants usually consist of chemically active surface groupings of molecules such as amino acids or sugar side chains and usually have specific three-dimensional structural characteristics, as well as specific charge characteristics.

The terms “polypeptide,” “peptide,” and “protein” are used interchangeably herein to refer to a polymer of amino acid residues. The terms also apply to amino acid polymers in which one or more amino acid residues are artificial chemical mimetics of a corresponding naturally occurring amino acids, as well as to naturally occurring amino acid polymers and non-naturally occurring amino acid polymers.

As used herein, the phrase “therapeutic agent” refers to an immune modulatory agent that is covalently bonded to an antibody construct as described herein. Thus, the terms “therapeutic agent” and “immune modulatory agent” can be used interchangeably here. The therapeutic agent can elicit the immune response (i.e., stimulation or suppression) while bonded to the antibody construct or after cleavage (e.g., enzymatic cleavage) from the antibody construct following administration of an immunoconjugate to the subject. The therapeutic agent can be an immune agonist or antagonist.

As used herein, the phrase “pattern recognition receptor” and term “PRR” refer to any member of a class of conserved mammalian proteins, which recognize pathogen-associated molecular patterns (PAMPs) or damage-associated molecular patterns (DAMPs), and act as key signaling elements in innate immunity. Pattern recognition receptors are divided into membrane-bound PRRs, cytoplasmic PRRs, and secreted PRRs. Examples of membrane-bound PRRs include Toll-like receptors (TLRs) and C-type lectin receptors (CLRs). Examples of cytoplasmic PRRs include NOD-like receptors (NLRs), such as NLRP3, Rig-I-like receptors (RLR), and STING (STimulator of INterferon Genes).

As used herein, the phrase “Toll-like receptor” and term “TLR” refer to any member of a family of highly conserved mammalian proteins, which recognize pathogen-associated molecular patterns and act as key signaling elements in innate immunity. TLR polypeptides share a characteristic structure that includes an extracellular domain that has leucine-rich repeats, a transmembrane domain, and an intracellular domain that is involved in TLR signaling.

The phrase “Toll-like receptor 1” and term “TLR1” refer to nucleic acids or polypeptides sharing at least 70%; 80%, 90%, 95%, 96%, 97%, 98%, 99%, or more sequence identity to a publicly-available TLR1 sequence, e.g., GenBank accession number AAY85643 for human TLR1 polypeptide, or GenBank accession number AAG37302 for murine TLR1 polypeptide.

The phrase “Toll-like receptor 2” and term “TLR2” refer to nucleic acids or polypeptides sharing at least 70%; 80%, 90%, 95%, 96%, 97%, 98%, 99%, or more sequence identity to a publicly-available TLR2 sequence, e.g., GenBank accession number AAY85648 for human TLR2 polypeptide, or GenBank accession number AAD49335 for murine TLR2 polypeptide.

The phrase “Toll-like receptor 3” and term “TLR3” refer to nucleic acids or polypeptides sharing at least 70%; 80%, 90%, 95%, 96%, 97%, 98%, 99%, or more sequence identity to a publicly-available TLR3 sequence, e.g., GenBank accession number AAC34134 for human TLR3 polypeptide, or GenBank accession number AAK26117 for murine TLR3 polypeptide.

The phrase “Toll-like receptor 4” and term “TLR4” refer to nucleic acids or polypeptides sharing at least 70%; 80%, 90%, 95%, 96%, 97%, 98%, 99%, or more sequence identity to a publicly-available TLR4 sequence, e.g., GenBank accession number AAY82270 for human TLR4 polypeptide, or GenBank accession number AAD29272 for murine TLR4 polypeptide.

The phrase “Toll-like receptor 5” and term “TLR5” refer to nucleic acids or polypeptides sharing at least 70%; 80%, 90%, 95%, 96%, 97%, 98%, 99%, or more sequence identity to a publicly-available TLR5 sequence, e.g., GenBank accession number ACM69034 for human TLR5 polypeptide, or GenBank accession number AAF65625 for murine TLR5 polypeptide.

The phrase “Toll-like receptor 6” and term “TLR6” refer to nucleic acids or polypeptides sharing at least 70%; 80%, 90%, 95%, 96%, 97%, 98%, 99%, or more sequence identity to a publicly-available TLR6 sequence, e.g., GenBank accession number ABY67133 for human TLR6 polypeptide, or GenBank accession number AAG38563 for murine TLR6 polypeptide.

The phrase “Toll-like receptor 7” and term “TLR7” refer to nucleic acids or polypeptides sharing at least 70%; 80%, 90%, 95%, 96%, 97%, 98%, 99%, or more sequence identity to a publicly-available TLR7 sequence, e.g., GenBank accession number AAZ99026 for human TLR7 polypeptide, or GenBank accession number AAK62676 for murine TLR7 polypeptide.

The phrase “Toll-like receptor 8” and term “TLR8” refer to nucleic acids or polypeptides sharing at least 70%; 80%, 90%, 95%, 96%, 97%, 98%, 99%, or more sequence identity to a publicly-available TLR8 sequence, e.g., GenBank accession number AAZ95441 for human TLR8 polypeptide, or GenBank accession number AAK62677 for murine TLR8 polypeptide.

The phrase “Toll-like receptor 7/8” and term “TLR7/8” refer to nucleic acids or polypeptides that are both TLR7 agonists and TLR8 agonists.

The phrase “Toll-like receptor 9” and term “TLR9” refer to nucleic acids or polypeptides sharing at least 70%; 80%, 90%, 95%, 96%, 97%, 98%, 99%, or more sequence identity to a publicly-available TLR9 sequence, e.g., GenBank accession number AAF78037 for human TLR9 polypeptide, or GenBank accession number AAK28488 for murine TLR9 polypeptide.

The phrase “Toll-like receptor 10” and term “TLR10” refer to nucleic acids or polypeptides sharing at least 70%; 80%, 90%, 95%, 96%, 97%, 98%, 99%, or more sequence identity to a publicly-available TLR10 sequence, e.g., GenBank accession number AAK26744 for human TLR10 polypeptide.

The phrase “Toll-like receptor 11” and term “TLR11” refer to nucleic acids or polypeptides sharing at least 70%; 80%, 90%, 95%, 96%, 97%, 98%, 99%, or more sequence identity to a publicly-available TLR11 sequence, e.g., GenBank accession number AAS83531 for murine TLR11 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as NK-κB, in the association of certain components (such as IRAK) with other proteins or intracellular structures, or in the biochemical activity of components such as kinases (such as MAPK).

As used herein, the term “amino acid” refers to any monomeric unit that can be incorporated into a peptide, polypeptide, or protein. Amino acids include naturally occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally occurring amino acid. Amino acids may be referred to herein by either the commonly known three letter symbols or by the one-letter symbols recommended by the IUPAC-IUB Biochemical Nomenclature Commission.

As used herein, the term “immune checkpoint inhibitors” refers to any modulator that inhibits the activity of the immune checkpoint molecule. Immune checkpoint inhibitors can include, but are not limited to, immune checkpoint molecule binding proteins, small molecule inhibitors, antibodies, antibody-derivatives (including Fc fusions, Fab fragments and scFvs), antibody-drug conjugates, antisense oligonucleotides, siRNA, aptamers, peptides and peptide mimetics.

Useful bonds for connecting linking moieties to proteins and other materials include, but are not limited to, amides, amines, esters, carbamates, ureas, thioethers, thiocarbamates, thiocarbonates, and thioureas. A “divalent” linking moiety contains two points of attachment for linking two functional groups; polyvalent linking moieties can have additional points of attachment for linking further functional groups. For example, divalent linking moieties include divalent polymer moieties such as divalent poly(ethylene glycol), divalent poly(propylene glycol), and divalent poly(vinyl alcohol).

As used herein, when the term “optionally present” is used to refer to a chemical structure (e.g., “X” or “Y”), if that chemical structure is not present, the bond originally made to the chemical structure is made directly to the adjacent atom.

As used herein, the term “linker” refers to a functional group that covalently bonds two or more moieties in a compound or material. For example, the linker can serve to covalently bond a therapeutic agent to an antibody construct in an immunoconjugate.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 30 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy. The term “alkylene” refers to a divalent alkyl radical.

As used herein, the term “heteroalkyl” refers to an alkyl group as described herein, wherein one or more carbon atoms are optionally and independently replaced with a heteroatom selected from N, O, and S. The term “heteroalkylene” refers to a divalent heteroalkyl radical.

As used herein, the term “cycloalkyl” refers to a saturated or partially unsaturated, monocyclic, fused bicyclic, or bridged polycyclic ring assembly containing from 3 to 12 ring atoms, or the number of atoms indicated. Cycloalkyls can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Cycloalkyl groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

Unsaturated carbocyclic groups also include aryl groups. The term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15, or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Aryl groups can be substituted or unsubstituted. “Substituted aryl” groups can be substituted with one or more groups selected from halo, hydroxy, amino, oxo (═O), alkylamino, amido, acyl, nitro, cyano, alkyl, and alkoxy.

A “divalent” cycloalkyl refers to a carbocyclic group having two points of attachment for covalently linking two moieties in a molecule or material. Cycloalkyl groups can be substituted or unsubstituted. “Substituted cycloalkyl” groups can be substituted with one or more groups selected from halo, hydroxy, amino, oxo (═O), alkylamino, amido, acyl, nitro, cyano, alkyl, and alkoxy.

As used herein, the term “heterocycle” refers to heterocycloalkyl groups and heteroaryl groups. “Heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O, or S. Additional heteroatoms can also be useful, including, but not limited to, B, Al, Si, and P. The heteroatoms can be oxidized to form moieties such as, but not limited to, —S(O)— and —S(O)₂-. Heteroaryl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. “Substituted heteroaryl” groups can be substituted with one or more groups selected from halo, hydroxy, amino, oxo (═O), alkylamino, amido, acyl, nitro, cyano, alkyl, and alkoxy.

As used herein, the term “aromatic moiety” refers to an aryl or heteroaryl group as described herein, which has been substituted as specified by the disclosure provided herein.

Heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

“Heterocycloalkyl,” by itself or as part of another substituent, refers to a saturated ring system having from 3 to 12 ring members and from 1 to 4 heteroatoms of N, O, and S. Additional heteroatoms can also be useful, including, but not limited to, B, Al, Si, and P. The heteroatoms can be oxidized to form moieties such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. “Substituted heterocycloalkyl” groups can be substituted with one or more groups selected from halo, hydroxy, amino, oxo (═O), alkylamino, amido, acyl, nitro, cyano, alkyl, and alkoxy.

Heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

As used herein, the terms “halo” and “halogen,” by themselves or as part of another substituent, refer to a fluorine, chlorine, bromine, or iodine atom.

As used herein, the term “carbonyl,” by itself or as part of another substituent, refers to —C(O)—, i.e., a carbon atom double-bonded to oxygen and bound to two other groups in the moiety having the carbonyl.

As used herein, the term “amino” refers to a moiety —NR₃, wherein each R group is H or alkyl. An amino moiety can be ionized to form the corresponding ammonium cation.

As used herein, the term “hydroxy” refers to the moiety —OH.

As used herein, the term “cyano” refers to a carbon atom triple-bonded to a nitrogen atom (i.e., the moiety —C≡N).

As used herein, the term “carboxy” refers to the moiety —C(O)OH. A carboxy moiety can be ionized to form the corresponding carboxylate anion.

As used herein, the term “amido” refers to a moiety —NRC(O)R or —C(O)NR₂, wherein each R group is H or alkyl.

As used herein, the term “nitro” refers to the moiety —NO₂.

As used herein, the term “oxo” refers to an oxygen atom that is double-bonded to a compound (i.e., O═).

As used herein, the phrase “salt” or “pharmaceutically acceptable salt” is intended to include salts derived from the parent compound which contains a basic or acidic moiety. Generally, such salts can be prepared by reacting the free acid or base forms of these compounds with a stoichiometric amount of the appropriate base or acid, respectively, in water or in an organic solvent, or in a mixture of the two. For example, an inorganic acid (e.g., hydrochloric acid, sulfuric acid, phosphoric acid, or hydrobromic acid), an organic acid (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base (e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typically utilized. Lists of suitable salts are found in Remington's Pharmaceutical Sciences, 18th ed., Mack Publishing Company, Easton, Pa., 1990, p. 1445, and Journal of Pharmaceutical Science, 66: 2-19 (1977). For example, suitable salts can be salts of alkali metals (e.g., sodium or potassium), alkaline earth metals (e.g., calcium), and ammonium.

As used herein, the terms “treat,” “treatment,” and “treating” refer to any indicia of success in the treatment or amelioration of an injury, pathology, condition, or symptom (e.g., cognitive impairment), including any objective or subjective parameter such as abatement; remission; diminishing of symptoms or making the symptom, injury, pathology or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

As used herein, the term “cancer” refers to conditions including solid cancers, lymphomas, and leukemias. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, stomach (gastric) cancer, cancer of the central nervous system, skin cancer (e.g., melanoma), choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma. Other types of cancer will be readily apparent from the disclosure provided herein.

As used herein, “disease or condition” refers to any disease or condition caused by or related to an autoimmune disease, inflammation, sepsis, allergy, asthma, graft rejection, graft-versus-host disease, immunodeficiency, or infectious disease (typically caused by an infectious pathogen, e.g., virus, bacteria, fungus, or parasite).

As used herein the phrases “effective amount” and “therapeutically effective amount” refer to a dose of a substance such as an immunoconjugate that produces one or more therapeutic effects for which the substance is administered. The particular dose will depend on the purpose of the treatment, and will be ascertainable by one skilled in the art using known techniques (see, e.g., Lieberman, Pharmaceutical Dosage Forms (volumes 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, ed., McGraw-Hill; and Remington: The Science and Practice of Pharmacy, 21st Edition, 2005, Hendrickson, Ed., Lippincott, Williams & Wilkins).

As used herein, the term “subject” refers to animals such as mammals, including, but not limited to, primates (e.g., humans), cows, sheep, goats, horses, dogs, cats, rabbits, rats, mice and the like. In certain embodiments, the subject is a human.

As used herein, the term “administering” refers to parenteral, intravenous, intraperitoneal, intramuscular, intratumoral, intralesional, intranasal or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a relatively close range surrounding that explicit value. If “X” were the value, “about X” or “around X” would indicate a value from 0.9X to 1.1X, e.g., from 0.95X to 1.05X or from 0.99X to 1.01X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.98X.”

Conjugation Method

The method for producing an immunoconjugate comprises combining one or more compounds of Formula I, or salts thereof, and an antibody construct of Formula II, or salt thereof, wherein Formula II is an antibody construct as described herein with residue

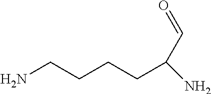

representing one or more lysine residues of the antibody construct, to provide the immunoconjugate of Formula III, or salt thereof: wherein TA is a therapeutic agent described herein, L is a linker described herein, r is an integer from 1 to 50, Ar is an aromatic moiety comprising a first substituent selected from PEG, —SO₂CX₃, —NR₃⁺, —NO₂, —SO₃R, —SO₂R, —CN, —CX₃, —PO₃R₂, —OPO₃R₂,

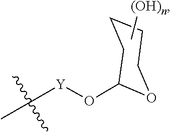

and salts thereof, each R independently is H, CX₃, or C₁-C₄ alkyl, each X independently is hydrogen or a halogen (e.g., —F, —Cl, —Br, or —I), Y is CH₂, PEG, or a bond, n is an integer from 1 to 4 (e.g., 1, 2, 3, or 4), and PEG has the formula: —(CH₂CH₂O)ₘ—(CH₂)ₚ—, where p is an integer from 1 to 5 (e.g., 1, 2, 3, 4, or 5) and m is an integer from 2 to 50 (e.g., 2 to 25, 2 to 10, or 2 to 6).

Ar is an aromatic moiety as described herein. Accordingly, Ar can be any aryl or heteroaryl group comprising a first substituent selected from PEG, —SO₂CX₃, —NR₃⁺, —NO₂, —SO₃R, —SO₂R, —CN, —CX₃, —PO₃R₂, —OPO₃R₂,

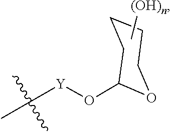

and salts thereof. In some embodiments, Ar is a phenyl, benzofuranyl, indoyl, or benzoimidazoyl group comprising a first substituent as described. In certain embodiments, Ar is a phenyl group comprising a first substituent as described.

The aromatic moiety comprises a first substituent selected from PEG, —SO₂CX₃, —NR₃⁺, —NO₂, —SO₃R, —SO₂R, —CN, —CX₃, —PO₃R₂, —OPO₃R₂,

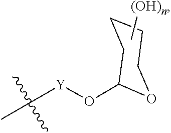

and salts thereof, each R independently is H, CX₃, or C₁-C₄ alkyl, each X independently is hydrogen or a halogen (e.g., —F, —Cl, —Br, or —I), Y is CH₂, PEG, or a bond, n is an integer from 1 to 4 (e.g., 1, 2, 3, or 4), and PEG has the formula: —(CH₂CH₂O)ₘ—(CH₂)ₚ—, where p is an integer from 1 to 5 (e.g., 1, 2, 3, 4, or 5), and m is an integer from 2 to 50 (e.g., 2 to 25, 2 to 10, or 2 to 6). In some embodiments, the first substituent is selected from —NO₂, —SO₃H, —CN, and salts thereof. In certain embodiments, the first substituent is —SO₃H or a salt thereof.

In some embodiments, the aromatic moiety (Ar) further comprises one or more additional substituents selected from —F, —Cl, —Br, —I, —CR₃, —OR, —C(O)R, —C(O)OR, (OH)˜PEG, —SO₂CX₃, —NR₃⁺, —NO₂, —SO₃R, —SO₂R, —CN, —CX₃, —PO₃R₂, —OPO₃R₂,

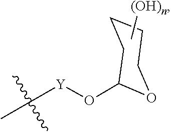

salts thereof, and combinations thereof, wherein each R independently is H, CX₃, or C₁-C₄ alkyl, each X independently is hydrogen or a halogen (e.g., —F, —Cl, —Br, or —I), Y is CH₂, PEG, or a bond, n is an integer from 1 to 4 (e.g., 1, 2, 3, or 4), and PEG has the formula: —(CH₂CH₂O)ₘ—(CH₂)ₚ—, where p is an integer from 1 to 5 (e.g., 1, 2, 3, 4, or 5) and m is an integer from 2 to 50 (e.g., 2 to 25, 2 to 10, or 2 to 6). The aromatic moiety can further comprise any number of additional substituents. For example, the aromatic moiety can further comprise from 1 to 10 additional substituents (e.g., 1, 2, 3, 4, 5, 6, 7, 8, 9, or 10), from 1 to 4 additional substituents (e.g., 1, 2, 3, or 4), or from 2 to 4 (e.g., 2, 3, or 4) additional substituents. In certain embodiments, the one or more additional substituents is selected from —F, —Cl, —Br, —I, —NO₂, —SO₃H, —CN, and salts thereof. In preferred embodiments, the one or more additional substituents is selected from —F, —Cl, —Br, and —I.

In some embodiments, Ar is of one of the following formulas Ar1 to Ar90:

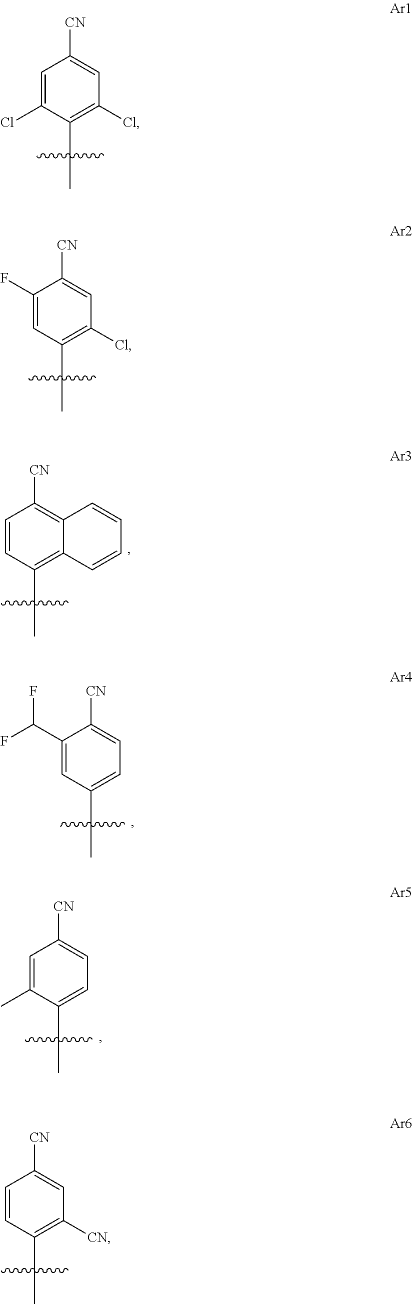

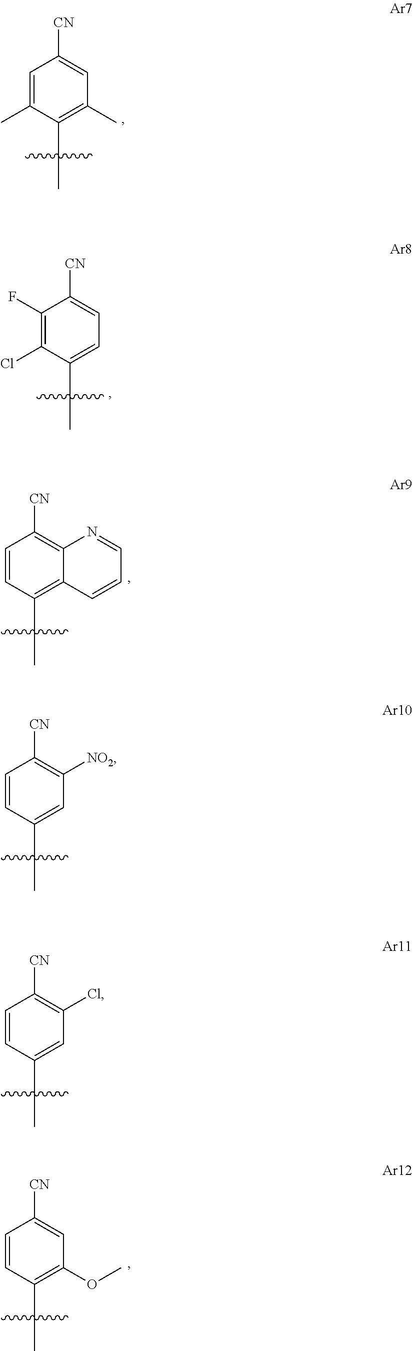

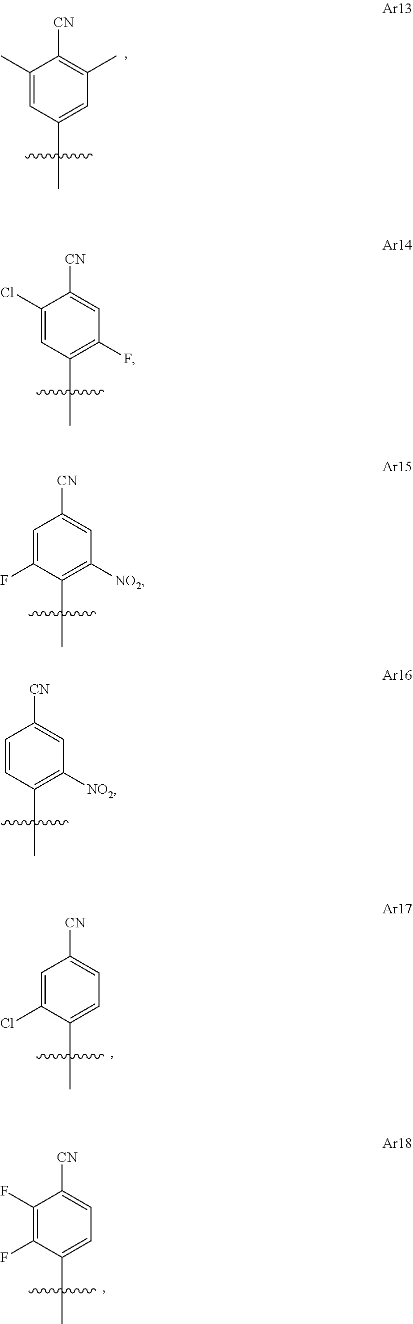

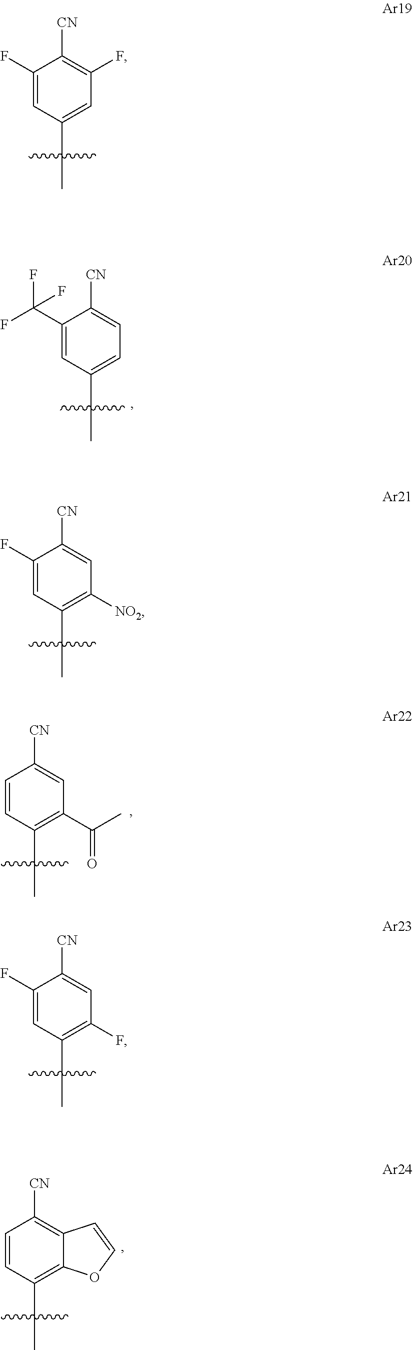 [SMILES: *c1cc(F)c(C#N)c(F)c1 ; *c1ccc(C#N)c(C(F)(F)F)c1 ; *c1cc(F)c(C#N)cc1[N+](=O)[O-] ; *c1ccc(C#N)cc1C(C)=O ; *c1cc(F)c(C#N)cc1F ; *c1ccc(C#N)c2ccoc12]

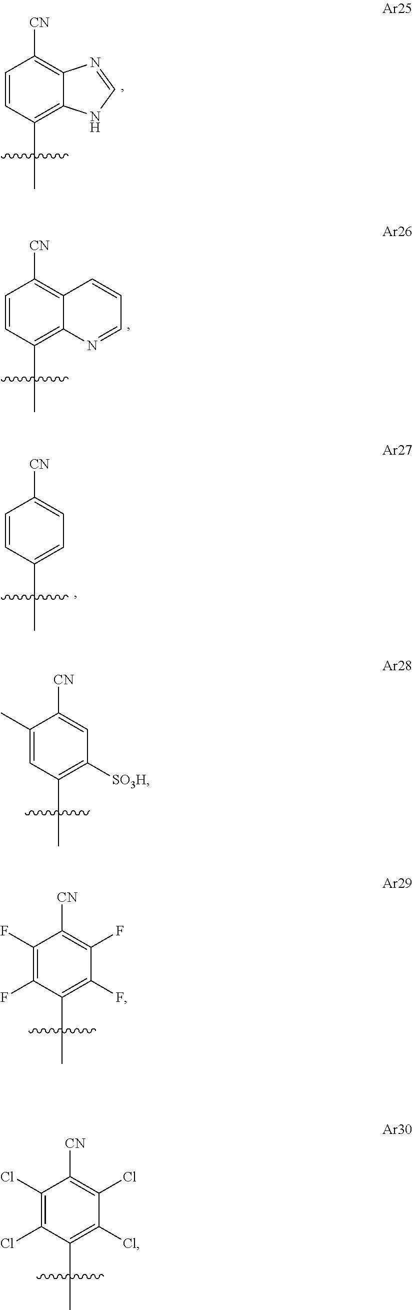 [SMILES: *c1ccc(C#N)c2nc[nH]c12 ; *c1ccc(C#N)c2cccnc12 ; *c1ccc(C#N)cc1 ; *c1cc(C)c(C#N)cc1S(=O)(=O)O ; *c1c(F)c(F)c(C#N)c(F)c1F ; *c1c(Cl)c(Cl)c(C#N)c(Cl)c1Cl]

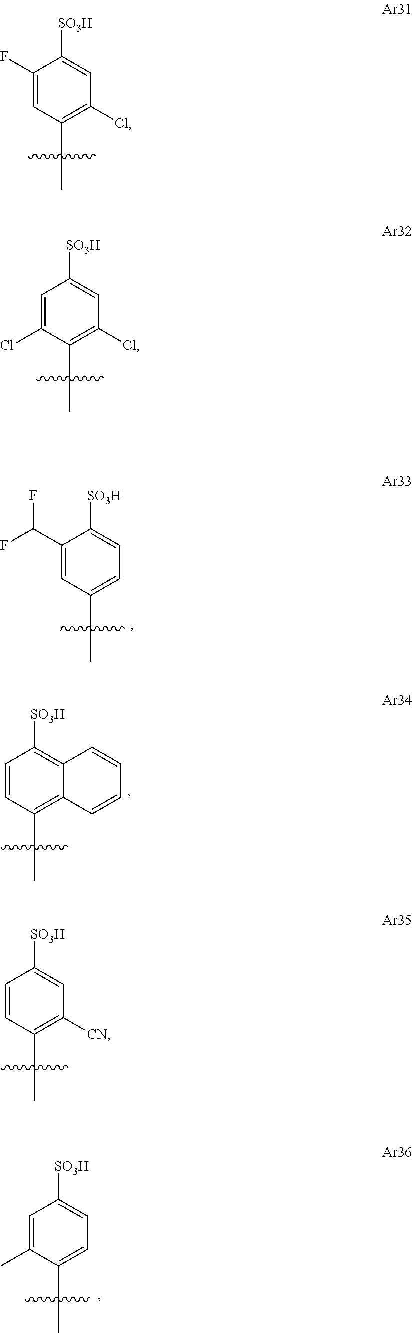 [SMILES: *c1cc(F)c(S(=O)(=O)O)cc1Cl ; *c1c(Cl)cc(S(=O)(=O)O)cc1Cl ; *c1ccc(S(=O)(=O)O)c(C(F)F)c1 ; *c1ccc(S(=O)(=O)O)c2ccccc12 ; *c1ccc(S(=O)(=O)O)cc1C#N ; *c1ccc(S(=O)(=O)O)cc1C]

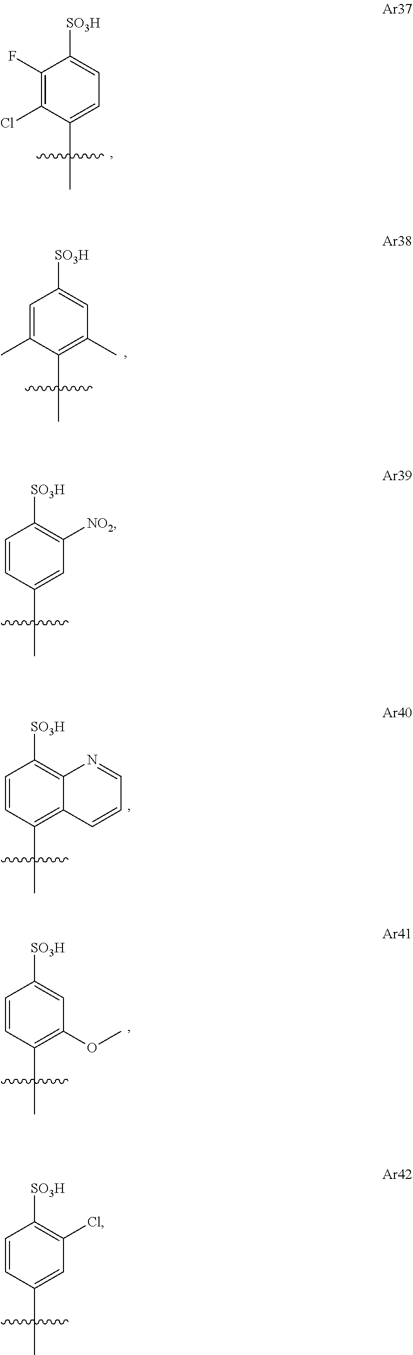

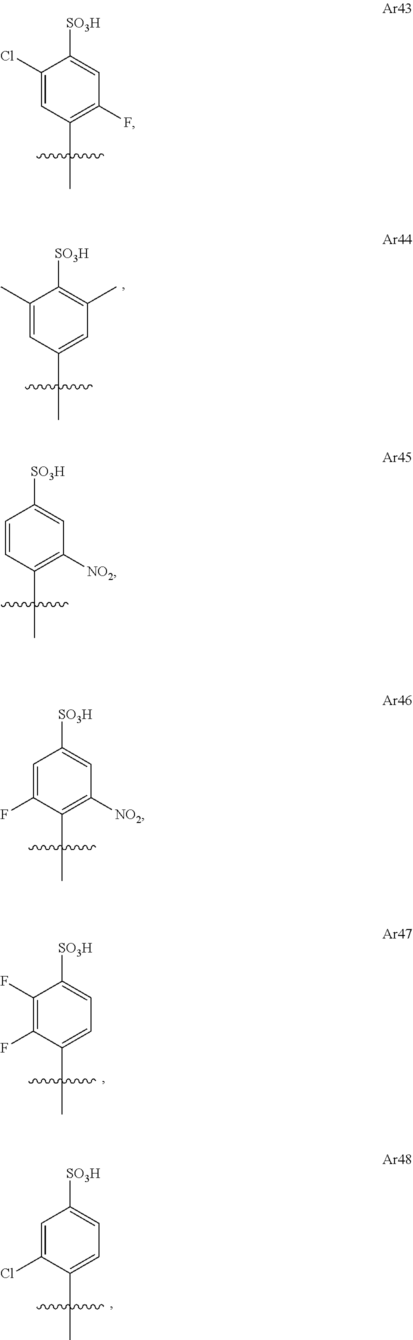

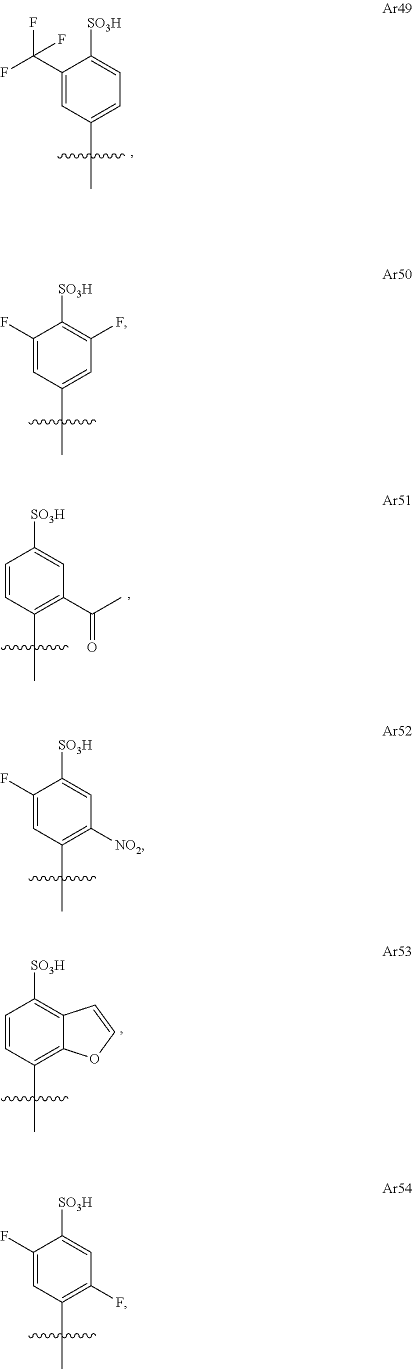 [SMILES: *c1ccc(S(=O)(=O)O)c(C(F)(F)F)c1 ; *c1cc(F)c(S(=O)(=O)O)c(F)c1 ; *c1ccc(S(=O)(=O)O)cc1C(C)=O ; *c1cc(F)c(S(=O)(=O)O)cc1[N+](=O)[O-] ; *c1ccc(S(=O)(=O)O)c2ccoc12 ; *c1cc(F)c(S(=O)(=O)O)cc1F]

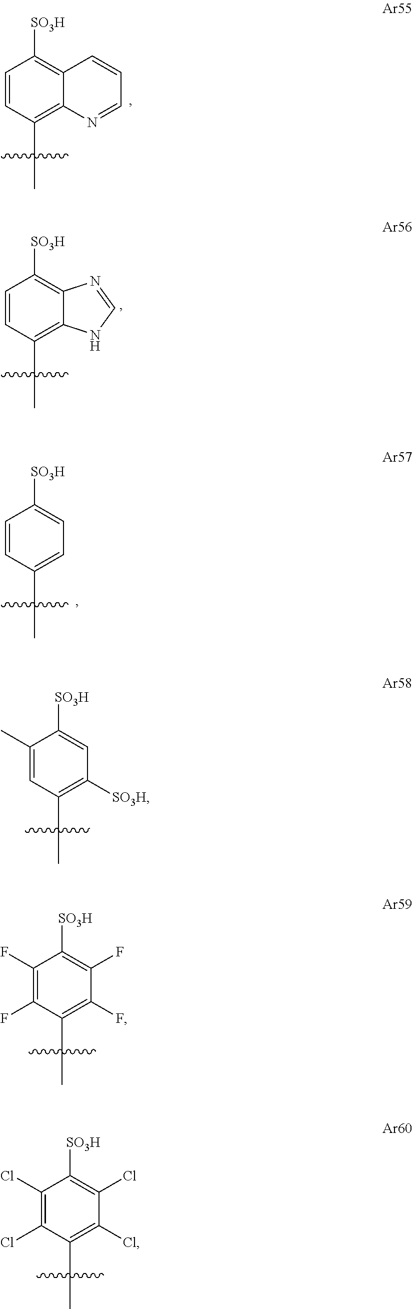 [SMILES: *c1ccc(S(=O)(=O)O)c2cccnc12 ; *c1ccc(S(=O)(=O)O)c2nc[nH]c12 ; *c1ccc(S(=O)(=O)O)cc1 ; *c1cc(C)c(S(=O)(=O)O)cc1S(=O)(=O)O ; *c1c(F)c(F)c(S(=O)(=O)O)c(F)c1F ; *c1c(Cl)c(Cl)c(S(=O)(=O)O)c(Cl)c1Cl]

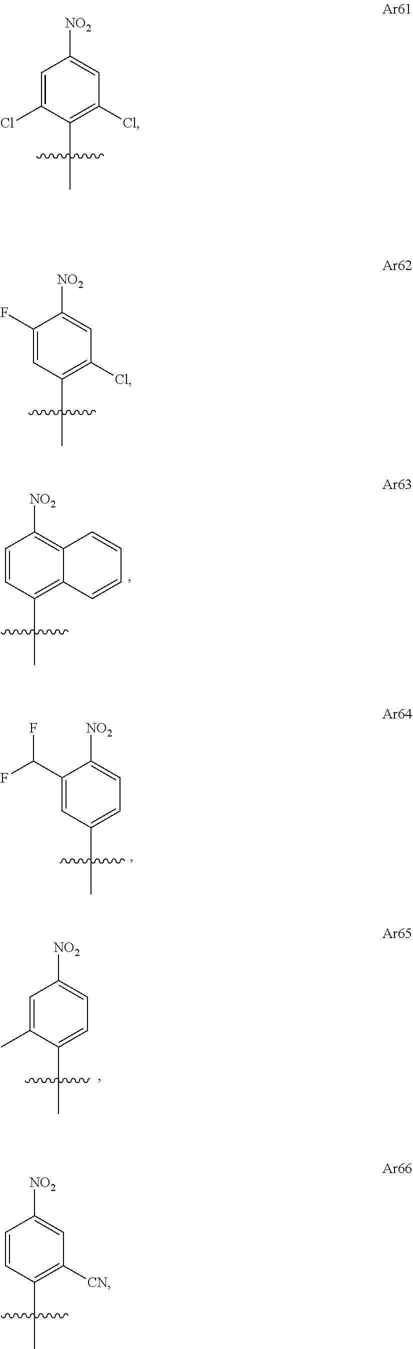 [SMILES: *c1c(Cl)cc([N+](=O)[O-])cc1Cl ; *c1cc(F)c([N+](=O)[O-])cc1Cl ; *c1ccc([N+](=O)[O-])c2ccccc12 ; *c1ccc([N+](=O)[O-])c(C(F)F)c1 ; *c1ccc([N+](=O)[O-])cc1C ; *c1ccc([N+](=O)[O-])cc1C#N]

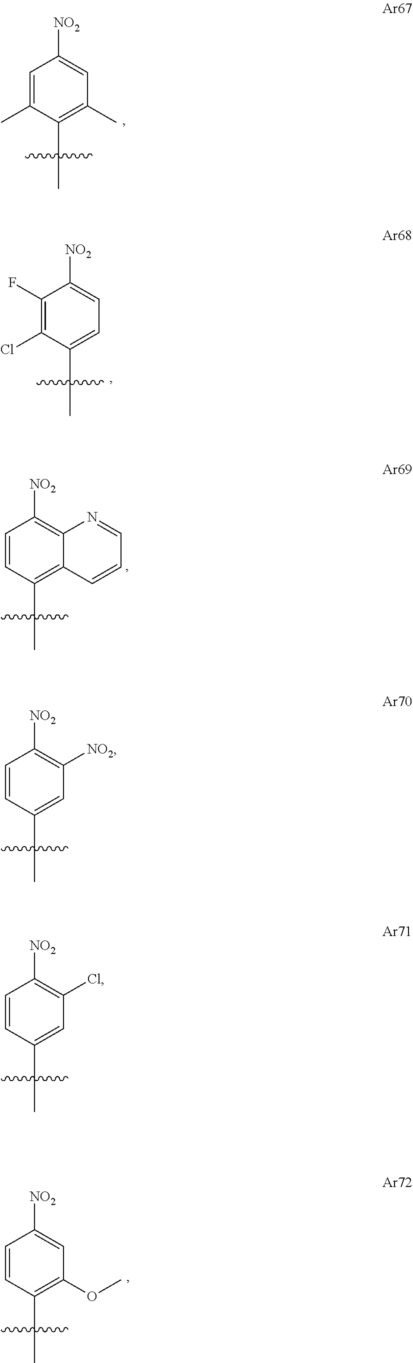

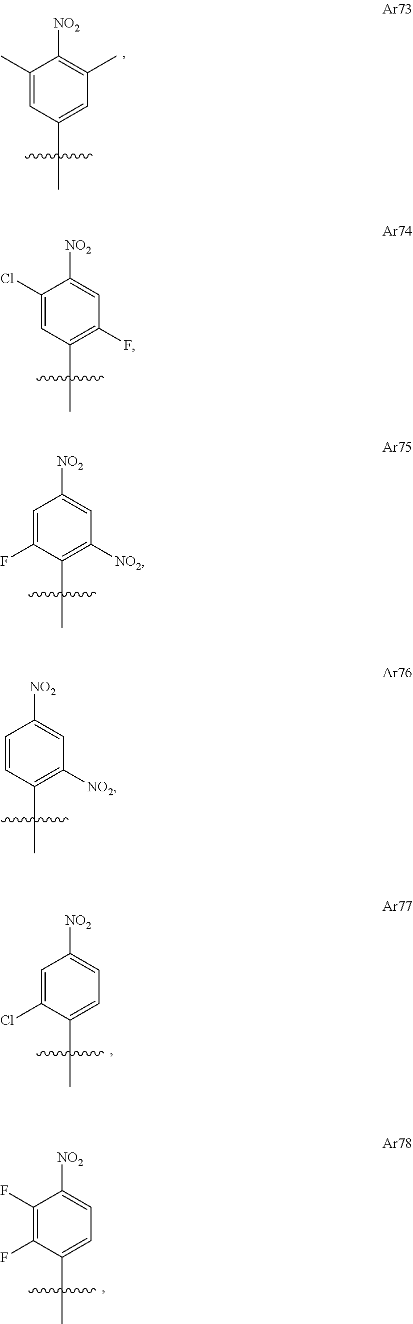

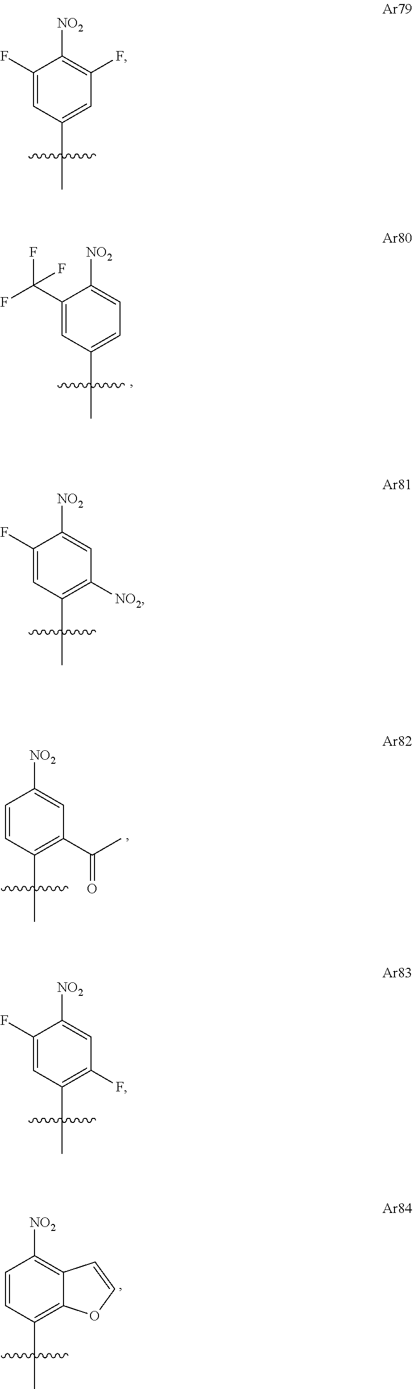 [SMILES: *c1cc(F)c([N+](=O)[O-])c(F)c1 ; *c1ccc([N+](=O)[O-])c(C(F)(F)F)c1 ; *c1cc(F)c([N+](=O)[O-])cc1[N+](=O)[O-] ; *c1ccc([N+](=O)[O-])cc1C(C)=O ; *c1cc(F)c([N+](=O)[O-])cc1F ; *c1ccc([N+](=O)[O-])c2ccoc12]

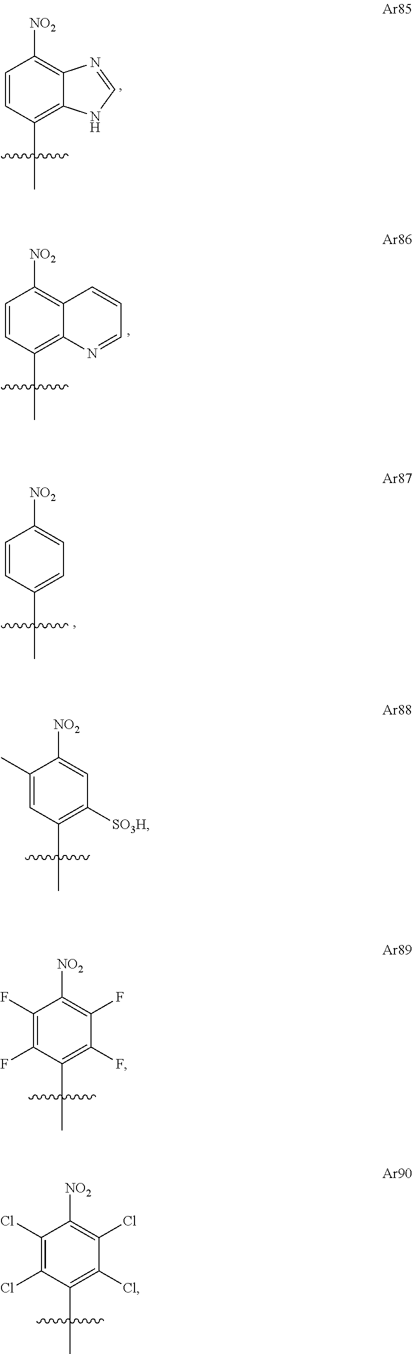 [SMILES: *c1ccc([N+](=O)[O-])c2nc[nH]c12 ; *c1ccc([N+](=O)[O-])c2cccnc12 ; *c1ccc([N+](=O)[O-])cc1 ; *c1cc(C)c([N+](=O)[O-])cc1S(=O)(=O)O ; *c1c(F)c(F)c([N+](=O)[O-])c(F)c1F ; *c1c(Cl)c(Cl)c([N+](=O)[O-])c(Cl)c1Cl]

or salts thereof. In certain embodiments, Ar is of formula Ar32, Ar59, or salts thereof (e.g., the lithium salt, the sodium salt, the potassium salt, or the ammonium salt).

In some embodiments, the method comprises combining the one or more compounds of Formula I, or salts thereof, and the antibody construct of Formula II, or salt thereof, until at least about 33 mol % (e.g., at least about 35 mol %, at least about 36 mol %, at least about 37 mol %, at least about 38 mol %, at least about 39 mol %, at least about 40 mol %, at least about 41 mol %, at least about 42 mol %, at least about 43 mol %, at least about 44 mol %, at least about 45 mol %, at least about 46 mol %, at least about 47 mol %, at least about 48 mol %, at least about 49 mol %, or at least about 50 mol %) of the one or more compounds of Formula I, or salts thereof, is conjugated to the antibody construct of Formula II, or salt thereof, to provide the immunoconjugate of Formula III, or salt thereof. In certain embodiments, the method comprises combining the one or more compounds of Formula I, or salts thereof, and the antibody construct of Formula II, or salt thereof, in the aqueous solution until at least 40 mol % of the one or more compounds of Formula I, or salts thereof, is conjugated to the antibody construct of Formula II, or salt thereof, to provide the immunoconjugate of Formula III, or salt thereof. In other embodiments, the method comprises combining the one or more compounds of Formula I, or salts thereof, and the antibody construct of Formula II, or salt thereof, in the aqueous solution until at least 50 mol % of the one or more compounds of Formula I, or salts thereof, is conjugated to the antibody construct of Formula II, or salt thereof, to provide the immunoconjugate of Formula III, or salt thereof.

In some embodiments, the method comprises combining the one or more compounds of Formula I, or salts thereof, and the antibody construct of Formula II, or salt thereof, for a period of at least about 1 hour (e.g., at least about 2 hours, at least about 3 hours, at least about 4 hours, at least about 5 hour, at least about 6 hours, at least about 8 hours, at least about 10 hours, at least about 12 hours, at least about 16 hours, at least about 20 hours, at least about 24 hours, or at least about 48 hours). Alternatively, or in addition, the method comprises combining the one or more compounds of Formula I, or salts thereof, and the antibody construct of Formula II, or salt thereof, for a period of not more than about 48 hours (e.g., not more than about 36 hours, not more than about 30 hours, not more than about 24 hours, not more than about 21 hours, not more than about 18 hour, not more than about 15 hours, or not more than about 12 hours). Thus, the method can comprise combining the one or more compounds of Formula I, or salts thereof, and the antibody construct of Formula II, or salt thereof, for a period bounded by any two of the aforementioned endpoints. For example, the method can comprise combining the one or more compounds of Formula I, or salts thereof, and the antibody construct of Formula II, or salt thereof, for a period of from about 1 hour to about 48 hours, from about 1 hour to about 36 hours, from about 1 hour to about 30 hours, from about 1 hour to about 24 hours, from about 1 hour to about 21 hours, from about 1 hour to about 18 hours, from about 1 hour to about 15 hours, from about 1 hour to about 12 hours, from about 2 hours to about 24 hours, from about 2 hours to about 15 hours, from about 2 hours to about 12 hours, from about 3 hours to about 24 hours, from about 3 hours to about 12 hours, from about 4 hours to about 24 hours, from about 5 hours to about 15 hours, from about 6 hours to about 48 hours, from about 6 hours to about 36 hours, from about 6 hours to about 30 hours, from about 6 hours to about 24 hours, from about 6 hours to about 21 hours, from about 6 hours to about 18 hours, from about 6 hours to about 15 hours, or from about 6 hours to about 12 hours.

In some embodiments, the method for producing an immunoconjugate of Formula III, or salt thereof, comprises combining the one or more compounds of Formula I, or salts thereof, and the antibody construct of Formula II, or salt thereof, in an alkaline aqueous solution (i.e., greater than a pH of 7). In certain embodiments, the method for producing an immunoconjugate of Formula III, or salt thereof, comprises combining the one or more compounds of Formula I, or salts thereof, and the antibody construct of Formula II, or salt thereof, in an aqueous solution that is buffered at a pH of about 7.5 to about 9, for example, about 7.6 to about 9, about 7.7 to about 9, about 7.8 to about to about 9, about 7.9 to about 9, about 8.0 to about 9, about 8 to about 8.9, about 8 to about 8.8, about 8 to about 8.7, about 8 to about 8.6, about 8.1 to about 8.6, about 8.2 to about 8.6, about 8.2 to about 8.5, or about 8.2 to about 8.4. Accordingly, the method for producing an immunoconjugate of Formula III, or salt thereof, can comprise combining the one or more compounds of Formula I, or salts thereof, and the antibody construct of Formula II, or salt thereof, in an aqueous solution that is buffered at a pH of about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In preferred embodiments, the method for producing an immunoconjugate of Formula III, or salt thereof, comprises combining the one or more compounds of Formula I, or salts thereof, and the antibody construct of Formula II, or salt thereof, in an aqueous solution that is buffered at a pH of about 8 to about 8.3.

The antibody construct of Formula II, or salt thereof, and the one or more compounds of Formula I, or salts thereof, can be combined in any suitable aqueous solution buffer such that the aqueous solution has an alkaline pH. An exemplary list of suitable aqueous solution buffers or first buffered aqueous solution is TES buffered saline, HEPES buffered saline, DIPSO buffered saline, MOBS buffered saline, acetamidoglycine buffered saline, TAPSO buffered saline, TEA buffered phosphate buffered saline, POPSO buffered saline, HEPPSO buffered saline, EPS buffered saline, HEPPS buffered saline, tricine buffered saline, glycinamide buffered saline, glycylglycine buffered saline, HEPBS buffered saline, bicine buffered saline, TAPS buffered saline, AMPB buffered saline, phosphate buffered saline, borate buffered saline, and tris buffered saline. In preferred embodiments, the aqueous solution buffer or first buffered aqueous solution is borate buffered saline. In another preferred embodiment, the aqueous solution buffer or first buffered aqueous solution is phosphate buffered saline.

In some embodiments, the aqueous solution buffer or first buffered aqueous solution further comprises a solubilizing agent. The solubilizing agent can be any compound (e.g., surfactant, dispersant, solvent, etc.) that improves the solubility of the compound of Formula I or salt thereof. For example, the solubilizing agent can be dimethylsulfoxide (DMSO), dimethyl acetamide (DMA), N-Methylpyrrolidone (NMP), ethylene glycol dimethyl ether, ethanol, methanol, or propylene glycol. In preferred embodiments, the solubilizing agent is DMA.

The solubilizing agent can be present in the aqueous buffer or first buffered aqueous solution in any suitable amount. For example, the solubilizing agent can be present in an amount from about 0.1 v/v % to about 40 v/v % of the aqueous buffer or first buffered aqueous solution, e.g., from about 0.1 v/v % to about 30 v/v %, from about 0.1 v/v % to about 20 v/v %, from about 1 v/v % to about 40 v/v %, from about 1 v/v % to about 30 v/v %, from about 1 v/v % to about 20 v/v %, from about 5 v/v % to about 40 v/v %, from about 5 v/v % to about 30 v/v %, or from about 5 v/v % to about 20 v/v %.

In some embodiments, the solubilizing agent increases the average therapeutic agent to antibody ratio of the immunoconjugate of Formula III or salt thereof. Accordingly, in some embodiments, the solubilizing agent increases the average therapeutic agent to antibody ratio of the immunoconjugate of Formula III, or salt thereof, as compared to a method without a solubilizing agent under otherwise identical reaction parameters. For example, a method using the solubilizing agent can produce an immunoconjugate of Formula III, or salt thereof, having an average therapeutic agent to antibody ratio of 0.2 or more (e.g., 0.4 or more, 0.6 or more, 0.8 or more, or 1 or more) greater than a method without a solubilizing agent under otherwise identical reaction parameters, as determined by liquid chromatography mass spectrometry analysis using a C4 reverse phase column on an ACQUITY™ UPLC H-class system (Waters Corporation) connected to a XEVO™ G2-XS TOF mass spectrometer (Waters Corporation).

In some embodiments, the solubilizing agent increases the yield of the immunoconjugate of Formula III or salt thereof. Accordingly, in some embodiments, the solubilizing agent increases the yield of the immunoconjugate of Formula III, or salt thereof, as compared to a method without a solubilizing agent under otherwise identical reaction parameters. For example, a method using the solubilizing agent can produce a 1% increase or more (e.g., 2% increase or more, 3% increase or more, 4% increase or more, 5% increase or more, or 10% increase or more) in yield of the immunoconjugate of Formula III, or salt thereof, than a method without a solubilizing agent under otherwise identical reaction parameters. The yield can be assessed by any suitable means, many of which are known to those skilled in the art.

The method for producing an immunoconjugate of Formula III, or salt thereof, comprises combining the one or more compounds of Formula I, or salts thereof, and the antibody construct of Formula II, or salt thereof, in an aqueous solution at any suitable temperature. In some embodiments, the method for producing an immunoconjugate of Formula III, or salt thereof, comprises combining the one or more compounds of Formula I, or salts thereof, and the antibody construct of Formula II, or salt thereof, in an aqueous solution at a temperature of about 0° C. to about 50° C., for example, about 0° C. to about 45° C., about 0° C. to about 40° C., about 5° C. to about to about 40° C., about 10° C. to about 40° C., about 15° C. to about 40° C., about 20° C. to about 40° C., about 25° C. to about 40° C., or about 25° C. to about 35° C. Accordingly, the method for producing an immunoconjugate of Formula III, or salt thereof, comprises combining the one or more compounds of Formula I, or salts thereof, and the antibody construct of Formula II, or salt thereof, in an aqueous solution at a temperature of about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In preferred embodiments, the method for producing an immunoconjugate of Formula III, or salt thereof, comprises combining the one or more compounds of Formula I, or salts thereof, and the antibody construct of Formula II, or salt thereof, in an aqueous solution at a temperature of about 30° C.

In some embodiments, the invention provides the immunoconjugate of Formula III, or salt thereof, in a first buffered aqueous solution. Typically, the first buffered aqueous solution is the same as the aqueous solution in which the antibody construct of Formula II, or salt thereof, and the one or more compounds of Formula I, or salts thereof, are combined. However, it will be understood by a person of ordinary skill in the art that the pH, temperature, and chemical composition of the first buffered aqueous solution may change slightly relative to the aqueous solution due to the combination of the antibody construct of Formula II, or salt thereof, and the one or more compounds of Formula I, or salts thereof, to form the immunoconjugate of Formula III, or salt thereof.

Any suitable number of equivalents of the one or more compounds of Formula I, or salts thereof, can be combined with the antibody construct of Formula II, or salt thereof, to achieve the desirable therapeutic agent to antibody ratio. Accordingly, about 0.1 equivalents or more of the one or more compounds of Formula I, or salts thereof, can be combined with the antibody construct of Formula II, or salt thereof, for example, about 0.5 equivalents or more, about 1 equivalent or more, about 1.5 equivalents or more, about 2 equivalents or more, about 2.5 equivalents or more, about 3 equivalents or more, about 3.5 equivalents or more, about 4 equivalents or more, about 4.5 equivalents or more, about 5 equivalents or more, about 5.5 equivalents or more, about 6 equivalents or more, about 6.5 equivalents or more, about 7 equivalents or more, about 7.5 equivalents or more, about 8 equivalents or more, about 8.5 equivalents or more, about 9 equivalents or more, about 9.5 equivalents or more, about 10 equivalents or more, about 11 equivalents or more, about 12 equivalents or more, about 13 equivalents or more, about 14 equivalents or more, about 15 equivalents or more, about 16 equivalents or more, about 17 equivalents or more, about 18 equivalents or more, about 19 equivalents or more, or about 20 equivalents or more. Alternatively, or in addition, about 50 equivalents or less of the one or more compounds of Formula I, or salts thereof, can be combined with the antibody construct of Formula II, or salt thereof, for example, about 45 equivalents or less, about 40 equivalent or less, about 35 equivalents or less, about 30 equivalents or less, about 25 equivalents or less, about 20 equivalents or less, about 18 equivalents or less, about 16 equivalents or less, about 14 equivalents or less, about 12 equivalents or less, about 10 equivalents or less, about 8 equivalents or less, about 6 equivalents or less, or about 4 equivalents or less. Thus, number of equivalents of the one or more compounds of Formula I, or salts thereof, combined with the antibody construct of Formula II, or salt thereof, can be bounded by any two of the aforementioned endpoints. For example, the number of equivalents of the one or more compounds of Formula I, or salts thereof, combined with the antibody construct of Formula II, or salt thereof, can be from about 0.1 to about 50, from about 1 to about 50, from about 1 to about 40, from about 1 to about 30, from about 1 to about 20, from about 2 to about 50, from about 2 to about 40, from about 2 to about 30, from about 2 to about 20, from about 3 to about 50, from about 3 to about 40, from about 3 to about 30, from about 3 to about 20, from about 4 to about 50, from about 4 to about 40, from about 4 to about 30, from about 4 to about 20, from about 6 to about 30, from about 6 to about 20, from about 8 to about 40, from about 8 to about 20, from about 10 to about 50, from about 10 to about 20, from about 12 to about 50, from about 12 to about 30, from about 12 to about 20, from about 4 to about 16, from about 8 to about 12, from about 1 to about 4, from about 1 to about 6, from about 1 to about 8, from about 1 to about 12, from about 1 to about 16, from about 2 to about 4, from about 2 to about 6, from about 2 to about 8, or from about 2 to about 12.

In some embodiments, the method further comprises purifying the immunoconjugate of Formula III, or salt thereof, in the first buffered aqueous solution and/or second buffered aqueous solution. Purification of the immunoconjugate of Formula III, or salt thereof, in the first buffered aqueous solution and/or second buffered aqueous solution can occur by any suitable means. For example, the immunoconjugate of Formula III, or salt thereof, in the first buffered aqueous solution and/or second buffered aqueous solution can be purified by column chromatography (e.g., anion exchange chromatography, cation exchange chromatography, hydrophobic interaction chromatography, or mixed-mode chromatography), centrifugation, filtration, or crystallization.

In some embodiments, the method for producing an immunoconjugate of Formula III, or salt thereof, comprises storing the immunoconjugate of Formula III, or salt thereof, at a lower pH than the pH at which the immunoconjugate was synthesized. Without wishing to be bound by any particular theory, it is believed that the immunoconjugate is more stable in neutral (i.e., a pH of about 6.5 to about 7.5) and/or acidic aqueous solutions (i.e., less than a pH of 7). Accordingly, the immunoconjugate of Formula III, or salt thereof, can be buffer exchanged to a second buffered aqueous solution that is buffered at a pH of about 7.5 or less, for example, about 7.4 or less, about 7.3 or less, about 7.2 or less, about 7.1 or less, about 7 or less, about 6.9 or less, about 6.8 or less, about 6.7 or less, about 6.6 or less, about 6.5 or less, about 6.4 or less, about 6.3 or less, about 6.2 or less, about 6.1 or less, or about 6 or less. In certain embodiments, the immunoconjugate of Formula III, or salt thereof, is synthesized in an alkaline first buffered aqueous solution, and stored in an acidic second buffered aqueous solution.

In some embodiments, the method further comprises performing a buffer exchange on the first buffered aqueous solution of the immunoconjugate of Formula III, or salt thereof, to provide a second buffered aqueous solution buffered at a pH of about 6 to about 7.5. In certain embodiments, the method further comprises performing a buffer exchange on the first buffered aqueous solution of the immunoconjugate of Formula III, or salt thereof, to provide a second buffered aqueous solution buffered at a pH of about 7 to about 7.5. In preferred embodiments, the method further comprises performing a buffer exchange on the first buffered aqueous solution of the immunoconjugate of Formula III, or salt thereof, to provide a second buffered aqueous solution buffered at a pH of about 7.2 to about 7.4.

The immunoconjugate of Formula III, or salt thereof, can be buffer exchanged to any suitable second aqueous solution buffer. In some embodiments, the second aqueous solution buffer is a neutral (i.e., a pH of about 6.5 to about 7.5) or acidic aqueous solution (i.e., less than a pH of 7). An exemplary list of suitable second aqueous solution buffers is MOPS buffered saline, cholamine chloride buffered saline, MOPSO buffered saline, ACES buffered saline, PIPES buffered saline, bis-tris propane buffered saline, ACES buffered saline, ADA buffered saline, bis-tris methane buffered saline, MES buffered saline, phosphate buffered saline, citrate buffered saline, and BES buffered saline. In preferred embodiments, the second aqueous solution is buffered with phosphate buffered saline.

In some embodiments, the aromatic moiety increases the solubility of the compound of Formula I or salt thereof. Accordingly, in some embodiments, the compound of Formula I, or salt thereof, has increased solubility in the aqueous buffer or first buffered aqueous solution as compared to an identical compound with tetrafluorophenyl as the aromatic moiety under otherwise identical reaction parameters. For example, the compound of Formula I, or salt thereof, can have a 1% reduction or more (e.g., 2% reduction or more, 3% reduction or more, 4% reduction or more, or 5% reduction or more) in turbidity relative to an identical compound with tetrafluorophenyl as the aromatic moiety under otherwise identical reaction parameters, as measured by the absorbance at 600 nm of a 0.1 M solution of the compound in a buffer containing 100 mM boric acid, 50 mM sodium chloride, and 1 mM ethylenediaminetetraacetic acid at pH 8.3.

In some embodiments, the aromatic moiety increases the stability of the compound of Formula I or salt thereof. Accordingly, in some embodiments, the compound of Formula I, or salt thereof, has increased stability in the aqueous buffer or first buffered aqueous solution as compared to an identical compound with succinimide in place of the aromatic moiety under otherwise identical reaction parameters. For example, the compound of Formula I, or salt thereof, can have a 1% reduction or more (e.g., 2% reduction or more, 3% reduction or more, 4% reduction or more, 5% reduction or more, or 10% reduction or more) in decomposition relative to an identical compound with succinimide in place of the aromatic moiety under otherwise identical reaction parameters, as measured by quantitative HPLC of a 0.1 M solution of the compound in a buffer containing 100 mM boric acid, 50 mM sodium chloride, and 1 mM ethylenediaminetetraacetic acid at pH 8.3, which has been prepared in a capped glass vial and incubated at 30° C. for 15 minutes.

Without wishing to be bound by any particular theory, it is believed that the combination of solubility, stability, and reactivity (i.e., as a result of the electronics and/or sterics) provided by the aromatic moiety beneficially impact one or more of reaction rate of the process, therapeutic agent to antibody ratio of the immunoconjugate of Formula III, yield of the immunoconjugate of Formula III, conjugation profile (i.e., the locations (amino acid residues) at which the therapeutic agent/linker is bound) of the immunoconjugate of Formula III, and purity of the immunoconjugate of Formula III.

In some embodiments, the aromatic moiety increases the rate (mol/L/s) of the formation of the immunoconjugate of Formula III, or salt thereof, using the steady state kinetics approximation. Accordingly, in some embodiments, the compound of Formula I, or salt thereof, increases the rate (mol/L/s) of formation of the immunoconjugate of Formula III, or salt thereof, as compared to a method using an identical compound with tetrafluorophenyl as the aromatic moiety under otherwise identical reaction parameters. For example, a method using the compound of Formula I, or salt thereof, can have a 1% increase or more (e.g., 2% increase or more, 3% increase or more, 4% increase or more, or 5% increase or more) in the rate (mol/L/s) of formation of the immunoconjugate of Formula III, or salt thereof, relative to a method using an identical compound with tetrafluorophenyl as the aromatic moiety under otherwise identical reaction parameters, as measured by steady state kinetics under otherwise identical reaction parameters.

In some embodiments, the aromatic moiety increases the average therapeutic agent to antibody ratio of the immunoconjugate of Formula III or salt thereof. Accordingly, in some embodiments, the compound of Formula I, or salt thereof, increases the average therapeutic agent to antibody ratio of the immunoconjugate of Formula III, or salt thereof, as compared to a method using an identical compound with tetrafluorophenyl as the aromatic moiety under otherwise identical reaction parameters. For example, a method using the compound of Formula I, or salt thereof, can produce an immunoconjugate of Formula III, or salt thereof, having an average therapeutic agent to antibody ratio of 0.2 or more (e.g., 0.4 or more, 0.6 or more, 0.8 or more, or 1 or more) greater than a method using an identical compound with tetrafluorophenyl as the aromatic moiety under otherwise identical reaction parameters, as determined by liquid chromatography mass spectrometry analysis using a C4 reverse phase column on an ACQUITY™ UPLC H-class system (Waters Corporation) connected to a XEVO™ G2-XS TOF mass spectrometer (Waters Corporation).

In some embodiments, the aromatic moiety increases the yield of the immunoconjugate of Formula III or salt thereof. Accordingly, in some embodiments, the compound of Formula I, or salt thereof, increases the yield of the immunoconjugate of Formula III, or salt thereof, as compared to a method using an identical compound with succinimide in place of the aromatic moiety under otherwise identical reaction parameters. For example, a method using the compound of Formula I, or salt thereof, can produce a 1% increase or more (e.g., 2% increase or more, 3% increase or more, 4% increase or more, 5% increase or more, or 10% increase or more) in yield of the immunoconjugate of Formula III, or salt thereof, than a method using an identical compound with succinimide in place of the aromatic moiety under otherwise identical reaction parameters. The yield can be assessed by any suitable means, many of which are known to those skilled in the art.

In some embodiments, the aromatic moiety modifies the conjugation profile of the immunoconjugate of Formula III or salt thereof. More particularly, the aromatic moiety can increase the amount of conjugation to the heavy chain of an antibody construct. Accordingly, in some embodiments, the compound of Formula I, or salt thereof, increases the amount of conjugation to the heavy chain of the antibody construct of the immunoconjugate of Formula III, or salt thereof, as compared to a method using an identical compound with tetrafluorophenyl as the aromatic moiety under otherwise identical reaction parameters. For example, a method using the compound of Formula I, or salt thereof, can increase the amount of conjugation to the heavy chain of the antibody construct of the immunoconjugate of Formula III, or salt thereof, by 5% or more (e.g., 10% or more, 15% or more, or 20% or more) relative to a method using an identical compound with tetrafluorophenyl as the aromatic moiety under otherwise identical reaction parameters. Peptide mapping of the light chain (LC) and the heavy chain (HC) can be carried out by injecting the reduced samples onto a C4 reverse phase column on an ACQUITY™ UPLC H-class system (Waters Corporation) connected to a XEVO™ G2-XS TOF mass spectrometer (Waters Corporation).

In some embodiments, the aromatic moiety increases the purity of the immunoconjugate of Formula III or salt thereof. Without wishing to be bound by any particular theory, it is believed that the increased solubility of the aromatic moiety allows for more efficient removal of the byproduct formed from the conjugation reaction (i.e., the resulting alcohol). Accordingly, in some embodiments, the compound of Formula I, or salt thereof, increases the purity of the immunoconjugate of Formula III, or salt thereof, as compared to a method using an identical compound with succinimide in place of the aromatic moiety under otherwise identical reaction parameters. For example, a method using the compound of Formula I, or salt thereof, can decrease the amount of detectable impurities present in the immunoconjugate of Formula III, or salt thereof, prior to column chromatography by 1% or more (e.g., 2% or more, 3% or more, 4% or more, or 5% or more) relative to a method using an identical compound with succinimide in place of the aromatic moiety under otherwise identical reaction parameters. The purity can be assessed by any means known by one of skill in the art.

Immunoconjugates

The methods described herein provide an immunoconjugate of Formula III:

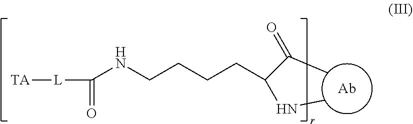

or salt thereof, wherein TA is a therapeutic agent described herein, L is a linker described herein, r is an integer from 1 to 50, and Ab is a portion of the antibody construct described herein. In some embodiments, the therapeutic agents, as defined by variable r, per immunoconjugate ranges from about 1 to about 10, for example, from about 1 to about 8, or from about 1 to about 6, or from about 1 to about 4. In some embodiments, the number of therapeutic agents per immunoconjugate is 1, 2, 3, 4, 5, or 6. In some embodiments, the number of therapeutic agents per immunoconjugate is 2. In some cases, the antibody construct is covalently bonded to a single therapeutic agent via a linker. In some cases, the antibody construct is covalently bonded to 2 or more therapeutic agents (e.g., 3 or more, 4 or more, or 5 or more therapeutic agents) via a linker. In some cases, the antibody construct is covalently bonded to 1-8 therapeutic agents (e.g., 1-5, 1-3, 2-8, 2-5, 2-3, or 3-8 therapeutic agents) via a linker. In some cases, the antibody construct is covalently bonded to 2-8 therapeutic agents (e.g., 2-5, 2-3, or 3-8 therapeutic agents).

It will be readily understood to a person skilled in the art that, while the methods described herein provide an immunoconjugate of Formula III, or any other immunoconjugate described herein, the methods also can provide a composition comprising a plurality of immunoconjugates of Formula III, or any other immunoconjugate described herein. In other words, the methods described herein generally provide a distribution of immunoconjugates such that the average number of therapeutic agents per immunoconjugate (i.e., the average of variable r in the definition of Formula III) ranges from about 1 to about 50. The average number of therapeutic agents per immunoconjugate can range, for example, from about 1 to about 10, from about 1 to about 8, or from about 1 to about 6, or from about 1 to about 4. The average number of therapeutic agents per immunoconjugate can be about 0.8, 1, 1.2, 1.4, 1.6, 1.8, 2, 2.2, 2.4, 2.6, 2.8, 3, 3.2, 3.4, 3.6, 3.8, 4.0, or 4.2. In some embodiments, the average number of therapeutic agents per immunoconjugate is about 4. In some embodiments, the average number of therapeutic agents per immunoconjugate is about 2.

Thus, the methods described herein can also provide a composition comprising a plurality of immunoconjugates of Formula III:

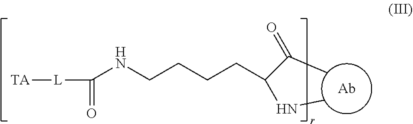

or salt thereof, wherein TA is a therapeutic agent described herein, L is a linker described herein, r is from about 1 to about 50, and Ab is a portion of the antibody construct described herein.

The method results in a therapeutic agent bound to the antibody construct via a linker at one or more lysine residues of the antibody construct. In embodiments, where the antibody construct is an IgG antibody, the therapeutic agent can be bound to the IgG at one or more locations on the light chain of the antibody, the heavy chain of the antibody, or a combination thereof. More particularly, the therapeutic agent can be bound at one or more of K¹⁰³, K¹⁰⁷, K¹⁴⁹, K¹⁶⁹, K¹⁸³, and K¹⁸⁸ of the light chain and/or one or more of K³⁰, K⁴³, K⁶⁵, K⁷⁶, K¹³⁶, K²¹⁶, K²¹⁷, K²²⁵, K²⁹³, K³²⁰, K³²³, K³³⁷, K³⁹⁵, and K⁴¹⁷ of the heavy chain according to Kabat numbering.

In some embodiments where the antibody construct is an IgG antibody, the method results in a plurality of immunoconjugates with the therapeutic agent bound to the IgG antibody at one or more locations (i.e., lysine residues). In such instances, the method can result in a composition comprising a plurality of immunoconjugates of Formula III, or salts thereof, said plurality of immunoconjugates of Formula III, or salts thereof, having greater than 60% (e.g., greater than 65% or greater than 70%) of the therapeutic agents bound to the heavy chain via a linker. In preferred embodiments, where the antibody construct is an IgG antibody, the method results in a composition comprising a plurality of immunoconjugates of Formula III, or salts thereof, said plurality of immunoconjugates of Formula III, or salts thereof, having greater than 65% of the therapeutic agents bound to the heavy chain via a linker. In some embodiments, where the antibody construct is an IgG antibody, the method results in a composition comprising a plurality of immunoconjugates of Formula III, or salts thereof, said plurality of immunoconjugates of Formula III, or salts thereof, having greater than 5% of the therapeutic agents bound via a linker to one or more of K⁴³, K⁶⁵, and K⁴¹⁷ of the heavy chain. In certain embodiments, where the antibody construct is an IgG antibody, the method results in a composition comprising a plurality of immunoconjugates of Formula III, or salts thereof, said plurality of immunoconjugates of Formula III, or salts thereof, having greater than 5% of the therapeutic agents bound via a linker to each of K⁴³, K⁶⁵, and K⁴¹⁷ of the heavy chain.

Other characteristics of the immunoconjugate of Formula III (e.g., antibody constructs, therapeutic agents, linkers, and compositions) will be readily apparent from the disclosure provided herein.

Antibody Constructs

Generally, the immunoconjugates of the invention comprise an antibody construct comprising (i) an antigen binding domain and (ii) an Fc domain. In some embodiments, the antibody construct further comprises a targeting binding domain. In certain embodiments, the antibody construct is an antibody. In certain embodiments, the antibody construct is a fusion protein.

The antibodies in the immunoconjugates can be allogeneic antibodies. The phrase “allogeneic antibody” and term “alloantibody” refer to an antibody that is not from the individual in question (e.g., an individual with a tumor and seeking treatment), but is from the same species, or is from a different species, but has been engineered to reduce, mitigate, or avoid recognition as a xeno-antibody (e.g., non-self). For example, the “allogeneic antibody” can be a humanized antibody. One skilled in the art is knowledgeable regarding how to engineer a non-human antibody to avoid recognition as a xeno-antibody. Unless specifically stated otherwise, “antibody” and “allogeneic antibodies” as used herein refer to immunoglobulin G (IgG) or immunoglobulin A (IgA).

If a cancer cell of a human individual is contacted with an antibody that was not generated by that same person (e.g., the antibody was generated by a second human individual, the antibody was generated by another species such as a mouse, the antibody is a humanized antibody that was generated by another species, etc.), then the antibody is considered to be allogeneic (relative to the first individual). A humanized mouse monoclonal antibody that recognizes a human antigen (e.g., a cancer-specific antigen, an antigen that is enriched in and/or on cancer cells, etc.) is considered to be an “alloantibody” (an allogeneic antibody).

In some embodiments, the antibody is a polyclonal allogeneic IgG antibody. In some embodiments, the antibody is present in a mixture of polyclonal IgG antibodies with a plurality of binding specificities. In some embodiments, the antibodies of the mixture specifically bind to different target molecules, and in some cases, the antibodies of the mixture specifically bind to different epitopes of the same target molecule. Thus, a mixture of antibodies can in some cases include more than one immunoconjugate of the invention (e.g., therapeutic agents can be covalently bonded to antibodies of a mixture, e.g., a mixture of polyclonal IgG antibodies, resulting in a mixture of immunoconjugates of the invention). A mixture of antibodies can be pooled from 2 or more individuals (e.g., 3 or more individuals, 4 or more individuals, 5 or more individuals, 6 or more individuals, 7 or more individuals, 8 or more individuals, 9 or more individuals, 10 or more individuals, etc.). In some cases, pooled serum is used as a source of alloantibody, where the serum can come from any number of individuals, none of whom are the first individual (e.g., the serum can be pooled from 2 or more individuals, 3 or more individuals, 4 or more individuals, 5 or more individuals, 6 or more individuals, 7 or more individuals, 8 or more individuals, 9 or more individuals, 10 or more individuals, etc.). In some cases, the antibodies are isolated or purified from serum prior to use. The purification can be conducted before or after pooling the antibodies from different individuals.

In some cases where the antibodies in the immunoconjugates comprise IgGs from serum, the target antigens for some (e.g., greater than 0% but less than 50%), half, most (greater than 50% but less than 100%), or even all of the antibodies (i.e., IgGs from the serum) will be unknown. However, the chances are high that at least one antibody in the mixture will recognize the target antigen of interest because such a mixture contains a wide variety of antibodies specific for a wide variety of target antigens.

In some embodiments, the antibody is a polyclonal allogeneic IgA antibody. In some embodiments, the antibody is present in a mixture of polyclonal IgA antibodies with a plurality of binding specificities. In some cases, the antibodies of the mixture specifically bind to different target molecules, and in some cases the antibodies of the mixture specifically bind to different epitopes of the same target molecule. Thus, a mixture of antibodies can in some cases include more than one immunoconjugate of the invention (e.g., therapeutic agents can be covalently bonded to antibodies of a mixture, e.g., a mixture of polyclonal IgA antibodies, resulting in a mixture of immunoconjugates of the invention). A mixture of antibodies can be pooled from 2 or more individuals (e.g., 3 or more individuals, 4 or more individuals, 5 or more individuals, 6 or more individuals, 7 or more individuals, 8 or more individuals, 9 or more individuals, 10 or more individuals, etc.). In some cases, pooled serum is used as a source of alloantibody, where the serum can come from any number of individuals, none of whom are the first individual (e.g., the serum can be pooled from 2 or more individuals, 3 or more individuals, 4 or more individuals, 5 or more individuals, 6 or more individuals, 7 or more individuals, 8 or more individuals, 9 or more individuals, 10 or more individuals, etc.). In some cases, the antibodies are isolated or purified from serum prior to use. The purification can be conducted before or after pooling the antibodies from different individuals.

In some cases where the antibodies in the immunoconjugates comprise IgAs from serum, the target antigens for some (e.g., greater than 0% but less than 50%), half, most (greater than 50% but less than 100%), or even all of the antibodies (i.e., IgAs from the serum) will be unknown. However, the chances are high that at least one antibody in the mixture will recognize the target antigen of interest because such a mixture contains a wide variety of antibodies specific for a wide variety of target antigens.

In some cases, the antibody in the immunoconjugates includes intravenous immunoglobulin (IVIG) and/or antibodies from (e.g., enriched from, purified from, e.g., affinity purified from) IVIG. IVIG is a blood product that contains IgG (immunoglobulin G) pooled from the plasma (e.g., in some cases without any other proteins) from many (e.g., sometimes over 1,000 to 60,000) normal and healthy blood donors. IVIG is commercially available. IVIG contains a high percentage of native human monomeric IVIG and has low IgA content. When administered intravenously, IVIG ameliorates several disease conditions. Therefore, the United States Food and Drug Administration (FDA) has approved the use of IVIG for a number of diseases including (1) Kawasaki disease; (2) immune-mediated thrombocytopenia; (3) primary immunodeficiencies; (4) hematopoietic stem cell transplantation (for those older than 20 years); (5) chronic B-cell lymphocytic leukemia; and (6) pediatric HIV type 1 infection. In 2004, the FDA approved the Cedars-Sinai IVIG Protocol for kidney transplant recipients so that such recipients could accept a living donor kidney from any healthy donor, regardless of blood type (ABO incompatible) or tissue match. These and other aspects of IVIG are described, for example, in U.S. Patent Application Publications 2010/0150942; 2004/0101909; 2013/0177574; 2013/0108619; and 2013/0011388; which are hereby incorporated by reference in their entireties.

In some cases, the antibody is a monoclonal antibody of a defined sub-class (e.g., IgG₁, IgG₂, IgG₃, IgG₄, IgA₁, or IgA₂). If combinations of antibodies are used, the antibodies can be from the same subclass or from different subclasses. For example, the antibodies can be IgG₁ antibodies. Various combinations of different subclasses, in different relative proportions, can be obtained by those of skill in the art. In some cases, a specific subclass, or a specific combination of different subclasses can be particularly effective at cancer treatment or tumor size reduction. Accordingly, some embodiments of the invention provide immunoconjugates wherein the antibody is a monoclonal antibody. In some embodiments, the monoclonal antibody is humanized.

In some embodiments, the antibody binds to an antigen of a cancer cell. For example, the antibody can bind to a target antigen that is present at an amount of at least 10; 100; 1,000; 10,000; 100,000; 1,000,000; 2.5×10⁶; 5×10⁶; or 1×10⁷ copies or more on the surface of a cancer cell.

In some embodiments, the antibody binds to an antigen on a cancer or immune cell at a higher affinity than a corresponding antigen on a non-cancer cell. For example, the antibody may preferentially recognize an antigen containing a polymorphism that is found on a cancer or immune cell as compared to recognition of a corresponding wild-type antigen on the non-cancer or non-immune cell. In some cases, the antibody binds a cancer or immune cell with greater avidity than a non-cancer or non-immune cell. For example, the cancer or immune cell can express a higher density of an antigen, thus providing for a higher affinity binding of a multivalent antibody to the cancer or immune cell.

In some cases, the antibody does not significantly bind non-cancer antigens (e.g., the antibody binds one or more non-cancer antigens with at least 10; 100; 1,000; 10,000; 100,000; or 1,000,000-fold lower affinity (higher Kd) than the target cancer antigen). In some cases, the target cancer antigen to which the antibody binds is enriched on the cancer cell. For example, the target cancer antigen can be present on the surface of the cancer cell at a level that is at least 2, 5, 10; 100; 1,000; 10,000; 100,000; or 1,000,000-fold higher than a corresponding non-cancer cell. In some cases, the corresponding non-cancer cell is a cell of the same tissue or origin that is not hyperproliferative or otherwise cancerous. In general, a subject IgG antibody that specifically binds to an antigen (a target antigen) of a cancer cell preferentially binds to that particular antigen relative to other available antigens. However, the target antigen need not be specific to the cancer cell or even enriched in cancer cells relative to other cells (e.g., the target antigen can be expressed by other cells). Thus, in the phrase “an antibody that specifically binds to an antigen of a cancer cell,” the term “specifically” refers to the specificity of the antibody and not to the uniqueness of the antigen in that particular cell type.

In some embodiments, the antibodies in the immunoconjugates contain a modified Fc region, wherein the modification modulates the binding of the Fc region to one or more Fc receptors.

In some embodiments, the antibody contains one or more modifications (e.g., amino acid insertion, deletion, and/or substitution) in the Fc region that result in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibody contains one or more modifications (e.g., amino acid insertion, deletion, and/or substitution) in the Fc region that reduce the binding of the Fc region of the antibody to FcγRIIB. In some embodiments, the antibody contains one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibody contains one of more modifications in the Fc region that increase the binding of the Fc region of the antibody to FcγRIIB. In some embodiments, the modifications substantially reduce or eliminate antibody effector functions.

In some embodiments, the modulated binding is provided by mutations in the Fc region of the antibody relative to the native Fc region of the antibody. The mutations can be in a CH₂ domain, a CH₃ domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody. Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (see, e.g., Jefferis et al., mAbs, 1(4): 332-338 (2009)).

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E233, G237, P238, H268, P271, L328 and A330. Additional Fc region modifications for modulating Fc receptor binding are described in, for example, U.S. Patent Application Publication 2016/0145350 and U.S. Pat. Nos. 7,416,726 and 5,624,821, which are hereby incorporated by reference in their entireties herein.

In some embodiments, the Fc region of the antibodies are modified to have an altered glycosylation pattern of the Fc region compared to the native non-modified Fc region.

Human immunoglobulin is glycosylated at the Asn297 residue in the Cy2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while defucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726 and U.S. Patent Application Publications 2007/0014795 and 2008/0286819, which are hereby incorporated by reference in their entireties.

In some embodiments, the antibodies are modified to contain an engineered Fab region with a non-naturally occurring glycosylation pattern. For example, hybridomas can be genetically engineered to secrete afucosylated mAb, desilylated mAb or deglycosylated Fc with specific mutations that enable increased FcRγIIIa binding and effector function. In some embodiments, the antibodies are engineered to be afucosylated.

In some embodiments, the entire Fc region of an antibody is exchanged with a different Fc region, so that the Fab region of the antibody is conjugated to a non-native Fc region. For example, the Fab region of atezolizumab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

In some embodiments, the Fc region is modified by attachment or inclusion of a transforming growth factor beta 1 (TGFβ1) receptor, or a fragment thereof, that is capable of binding TGFβ1. For example, the receptor can be TGFβ receptor II (TGFβRII) (see U.S. Pat. No. 9,676,863, which is incorporated herein in its entirety). In some embodiments, the TGFβ receptor is a human TGFβ receptor. In some embodiments, the Fc region (e.g., IgG) has a C-terminal fusion to a TGFβ receptor (e.g., TGFβRII) extracellular domain (ECD; e.g., amino acids 24-159 of SEQ ID NO: 9 of U.S. Pat. No. 9,676,863). An “Fc linker” may be used to attach the IgG to the TGFβR extracellular domain, for example, a G4S4G Fc linker. The Fc linker may be a short, flexible peptide that allows for the proper three-dimensional folding of the molecule while maintaining the binding-specificity to the targets. In some embodiments, the N-terminus of the TGFβ receptor is fused to the Fc region (with or without an Fc linker). In some embodiments, the C-terminus of the immunoglobulin heavy chain is fused to the TGFβ receptor (with or without an Fc linker). In some embodiments, the C-terminal lysine residue of the antibody heavy chain is mutated to alanine. In some embodiments, the antibody includes SEQ ID NO: 168.

Targets

In some embodiments, the antigen binding domain or antibody is capable of binding one or more targets or antigens selected from (e.g., specifically binds to a target selected from) 5T4, ABL, ABCF1, ACVR1, ACVR1B, ACVR2, ACVR2B, ACVRL1, ADORA2A, AFP, Aggrecan, AGR2, AICDA, AIF1, AIG1, AKAP1, AKAP2, ALCAM, ALK, AMH, AMHR2, AHMR2, ANGPT1, ANGPT2, ANGPTL3, ANGPTL4, ANPEP, APC, APOC1, AR, aromatase, ASPH, ATX, AX1, AXL, AZGP1 (zinc-a-glycoprotein), B4GALNT1, B7, B7.1, B7.2, B7-H1, B7-H3, B7-H4, B7-H6, BAD, BAFF, BAG1, BAI1, BCR, BCL2, BCL6, BCMA, BDNF, BLNK, BLR1 (MDR15), BIyS, BMP1, BMP2, BMP3B (GDFIO), BMP4, BMP6, BMP8, BMP10, BMPR1A, BMPR1B, BMPR2, BPAG1 (plectin), BRCA1, C19orflO (IL27w), C3, C4A, C5, C5R1, CA6, CA9, CANT1, CAPRIN-1, CASP1, CASP4, CAV1, CCBP2 (D6/JAB61), CCL1 (1-309), CCLI1 (eotaxin), CCL13 (MCP-4), CCL15 (MIP-Id), CCL16 (HCC-4), CCL17 (TARC), CCL18 (PARC), CCL19 (MIP-3b), CCL2 (MCP-1), MCAF, CCL20 (MIP-3a), CCL21 (MEP-2), SLC, exodus-2, CCL22(MDC/STC-I), CCL23 (MPIF-I), CCL24 (MPIF-2/eotaxin-2), CCL25 (TECK), CCL26(eotaxin-3), CCL27 (CTACK/ILC), CCL28, CCL3 (MIP-Ia), CCL4 (MIPIb), CCL5(RANTES), CCL7 (MCP-3), CCL8 (mcp-2), CCNA1, CCNA2, CCND1, CCNE1, CCNE2, CCR1 (CKR1/HM145), CCR2 (mcp-IRB/RA), CCR3 (CKR3/CMKBR3), CCR4, CCR5(CMKBR5/ChemR13), CCR6 (CMKBR6/CKR-L3/STRL22/DRY6), CCR7 (CKR7/EBI1), CCR8 or CDw198 (CMKBR8/TERI/CKR-L1), CCR9 (GPR-9-6), CCRL1 (VSHK1), CCRL2 (L-CCR), CD13, CD164, CD19, CDH6, CDIC, CD2, CD20, CD21, CD200, CD22, CD23, CD24, CD27, CD28, CD29, CD3, CD33, CD35, CD37, CD38, CD3E, CD3G, CD3Z, CD4, CD40, CD40L, CD44, CD45RB, CD47, CD52, CD56, CD69, CD70, CD72, CD74, CD79A, CD79B, CD8, CD80, CD81, CD83, CD86, CD97, CD99, CD117, CD125, CD137, CD147, CD179b, CD223, CD279, CD152, CD274, CDH1 (E-cadherin), CDH10, CDH12, CDH13, CDH18, CDH19, CDH2O, CDH3, CDH5, CDH7, CDH8, CDH9, CDH17, CDK2, CDK3, CDK4, CDK5, CDK6, CDK7, CDK9, CDKN1A (p21Wap1/Cip1), CDKN1B (p27Kip1), CDKN1C, CDKN2A (p16INK4a), CDKN2B, CDKN2C, CDKN3, CEA, CEACAM5, CEACAM6, CEBPB, CERI, CFC1B, CHGA, CHGB, Chitinase, CHST1O, CIK, CKLFSF2, CKLFSF3, CKLFSF4, CKLFSF5, CKLFSF6, CKLFSF7, CKLFSF8, CLDN3, CLDN6, CLDN7 (claudin-7), CLDN18, CLEC5A, CLEC6A, CLEC1IA, CLEC12A, CLEC14A, CLN3, CLU (clusterin), CMKLR1, CMKOR1 (RDC1), CNR1, C-MET, COL18A1, COLIA1, COL4A3, COL6A1, CR2, Cripto, CRP, CSF1 (M-CSF), CSF2 (GM-CSF), CSF3 (GCSF), CTAG1B (NY-ESO-1), CTLA4, CTL8, CTNNB1 (b-catenin), CTSB (cathepsin B), CX3CL1 (SCYD1), CX3CR1 (V28), CXCL1 (GRO1), CXCL1O (IP-IO), CXCLI1 (1-TAC/IP-9), CXCL12 (SDF1), CXCL13, CXCL14, CXCL16, CXCL2 (GRO2), CXCL3 (GRO3), CXCL5 (ENA-78/LIX), CXCL6 (GCP-2), CXCL9 (MIG), CXCR3 (GPR9/CKR-L2), CXCR4, CXCR6 (TYMSTR/STRL33/Bonzo), CYB5, CYC1, CYSLTR1, DAB2IP, DES, DKFZp451J0118, DLK1, DNCL1, DPP4, E2F1, Engel, Edge, Fennel, EFNA3, EFNB2, EGF, EGFR, ELAC2, ENG, Enola, ENO2, ENO3, ENPP3, EpCAM, EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, EPHB6, EPHRIN-A1, EPHRIN-A2, EPHRINA3, EPHRIN-A4, EPHRIN-A5, EPHRIN-A6, EPHRIN-B1, EPHRIN-B2, EPHRIN-B3, EPHB4, EPG, ERBB2 (HER-2), ERBB3, ERBB4, EREG, ERK8, Estrogen receptor, Earl, ESR2, F3 (TF), FADD, FAP, farnesyltransferase, FasL, FASNf, FCER1A, FCER2, FCGR3A, FGF, FGF1 (aFGF), FGF10, FGF1 1, FGF12, FGF12B, FGF13, FGF14, FGF16, FGF17, FGF18, FGF19, FGF2 (bFGF), FGF20, FGF21, FGF22, FGF23, FGF3 (int-2), FGF4 (HST), FGF5, FGF6 (HST-2), FGF7 (KGF), FGF8, FGF9, FGFR1, FGFR2, FGFR3, FGFR4, FIGF (VEGFD), FIL1(EPSILON), FBL1 (ZETA), FLJ12584, FLJ25530, FLRT1 (fibronectin), FLT1, FLT-3, FOLR1, FOLH1, FOS, FOSL1(FRA-1), FR-alpha, FY (DARC), GABRP (GABAa), GAGEB1, GAGEC1, GALNAC4S-6ST, GATA3, GD2, GD3, GDF5, GFI1, GFRA1, GGT1, GM-CSF, GNAS1, GNRH1, GPC1, GPC3, GPNB, GPR2 (CCR10), GPR31, GPR44, GPR81 (FKSG80), GRCC1O (C1O), GRP, GSN (Gelsolin), GSTP1, GUCY2C, HAVCR1, HAVCR2, HDAC, HDAC4, HDAC5, HDAC7A, HDAC9, Hedgehog, HER3, HGF, HIF1A, HIP1, histamine and histamine receptors, HLA-A, HLA-DR, HLA-DRA, HLA-E, HM74, HMOXI, HSP90, HUMCYT2A, ICEBERG, ICOSL, ID2, IFN-a, IFNA1, IFNA2, IFNA4, IFNA5, EFNA6, BFNA7, IFNB1, IFNgamma, IFNW1, IGBP1, IGF1, IGF1R, IGF2, IGFBP2, IGFBP3, IGFBP6, DL-1, ILIO, ILIORA, ILIORB, IL-1, IL1R1 (CD121a), IL1R2(CD121b), IL-IRA, IL-2, IL2RA (CD25), IL2RB(CD122), IL2RG(CD132), IL-4, IL-4R(CD123), IL-5, IL5RA(CD125), IL3RB(CD131), IL-6, IL6RA, (CD126), IR6RB(CD130), IL-7, IL7RA(CD127), IL-8, CXCR1 (IL8RA), CXCR2, (IL8RB/CD128), IL-9, IL9R(CD129), IL-10, IL10RA(CD210), IL10RB(CDW210B), IL-11, IL1IRA, IL-12, IL-12A, IL-12B, IL-12RB1, IL-12RB2, IL-13, IL13RA1, IL13RA2, IL14, IL15, IL15RA, IL16, IL17, IL17A, IL17B, IL17C, IL17R, IL18, IL18BP, IL18R1, IL18RAP, IL19, ILIA, ILIB, ILIF10, ILIF5, IL1F6, ILIF7, IL1F8, DL1F9, ILIHYI, ILIR1, IL1R2, ILIRAP, ILIRAPL1, ILIRAPL2, ILIRL1, IL1RL2, ILIRN, IL2, IL20, IL20RA, IL21R, IL22, IL22R, IL22RA2, IL23, DL24, IL25, IL26, IL27, IL28A, IL28B, IL29, IL2RA, IL2RB, IL2RG, IL3, IL30, IL3RA, IL4, 1L4, IL6ST (glycoprotein 130), ILK, INHA, INHBA, INSL3, INSL4, IRAK1, IRAK2, ITGA1, ITGA2, ITGA3, ITGA6 (α6 integrin), ITGAV, ITGB3, ITGB4 (β4 integrin), JAG1, JAK1, JAK3, JTB, JUN, K6HF, KAI1, KDR, KIT, KITLG, KLF5 (GC Box BP), KLF6, KLK10, KLK12, KLK13, KLK14, KLK15, KLK3, KLK4, KLK5, KLK6, KLK9, KRT1, KRT19 (Keratin 19), KRT2A, KRTHB6(hair-specific type II keratin), LICAM, LAG3, LAMA5, LAMP1, LEP (leptin), Lewis Y antigen (“LeY”), LILRB1, Lingo-p75, Lingo-Troy, LGALS3BP, LRRC15, LRP5, LRP6, LPS, LTA (TNF-b), LTB, LTB4R (GPR16), LTB4R2, LTBR, LY75, LYPD3, MACMARCKS, MAG or OMgp, MAGEA3, MAGEA6, MAP2K7 (c-Jun), MCP-1, MDK, MIB1, midkine, MIF, MISRII, MJP-2, MLSN, MK, MKI67 (Ki-67), MMP2, MMP9, MS4A1, MSMB, MT3 (metallothionectin-UI), mTOR, MTSS1, MUC1 (mucin), MUC16, MYC, MYD88, NCAM1, NCK2, NCR3LG1, neurocan, NFKB1, NFKB2, NGFB (NGF), NGFR, NgR-Lingo, NgRNogo66, (Nogo), NgR-p75, NgR-Troy, NMEI (NM23A), NOTCH, NOTCH1, NOTCH3, NOX5, NPPB, NROB1, NROB2, NRID1, NR1D2, NR1H2, NR1H3, NR1H4, NR112, NR113, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRP1, NRP2, NT5E, NTN4, NY-ESO1, ODZI, OPRDI, P2RX7, PAP, PART1, PATE, PAWR, P-cadherin, PCA3, PCD1, PD-L1, PCDGF, PCNA, PDGFA, PDGFB, PDGFRA, PDGFRB, PECAMI, L1-CAM, peg-asparaginase, PF4 (CXCL4), PGF, PGR, phosphacan, PIAS2, PI3 Kinase, PIK3CG, PLAU (uPA), PLG, PLXDCI, PKC, PKC-beta, PPBP (CXCL7), PPID, PR1, PRAME, PRKCQ, PRKD1, PRL, PROC, PROK2, PSAP, PSCA, PSMA, PTAFR, PTEN, PTHR2, PTGS2 (COX-2), PTK7, PTN, PVRIG, PVRL4, RAC2 (P21Rac2), RANK, RANK ligand, RARB, RGS1, RGS13, RGS3, RNFI1O (ZNF144), Ron, ROBO2, ROR1, RXR, S100A2, SCGB 1D2 (lipophilin B), SCGB2A1 (mammaglobin 2), SCGB2A2 (mammaglobin 1), SCYE1 (endothelial Monocyte-activating cytokine), SDF2, SERPENA1, SERPINA3, SERPINB5 (maspin), SERPINEI (PAI-I), SERPINFI, SHIP-1, SHIP-2, SHB1, SHB2, SHBG, SfcAZ, SLAMF7, SLC2A2, SLC33A1, SLC43A1, SLC44A4, SLC34A2, SLIT2, SLITRK6, SPP1, SPRR1B (Spr1), ST6GAL1, ST8SIA1, STAB1, STATE, STEAP, STEAP2, SSTR2, TACSTD2, TB4R2, TBX21, TCP1O, TDGF1, TEK, TGFA, TGFB1, TGFB1I1, TGFB2, TGFB3, TGFBI, TGFBR1, TGFBR2, TGFBR3, THIL, THBS1 (thrombospondin-1), THBS2, THBS4, THPO, TIE (Tie-1), TIMP3, tissue factor, TLR1, TLR2, TLR3, TLR4, TLR5, TLR6, TLR7, TLR8, TLR9, TLR10, TLR11, TNF, TNF-a, TNFAIP2 (B94), TNFAIP3, TNFRSFI1A, TNFRSF1A, TNFRSF1B, TNFRSF21, TNFRSF5, TNFRSF6 (Fas), TNFRSF7, TNFRSF8, TNFRSF9, TNFSF1O (TRAIL), TNFRSF10A, TNFRSF10B, TNFRSF12A, TNFRSF17, TNFSF1 1 (TRANCE), TNFSF12 (APO3L), TNFSF13 (April), TNFSF13B, TNFSF14 (HVEM-L), TNFRSF14 (HVEM), TNFSF15 (VEGI), TNFSF18, TNFSF4 (OX40 ligand), TNFSF5 (CD40 ligand), TNFSF6 (FasL), TNFSF7 (CD27 ligand), TNFSF8 (CD30 ligand), TNFSF9 (4-1BB ligand), TOLLIP, Toll-like receptors, TOP2A (topoisomerase Iia), TP53, TPM1, TPM2, TRADD, TRAF1, TRAF2, TRAF3, TRAF4, TRAF5, TRAF6, TRKA, TREM1, TREM2, TROP2, TRPC6, TSLP, TWEAK, Tyrosinase, uPAR, VEGF, VEGFB, VEGFC, versican, VHL C5, VLA-4, WT1, Wnt-1, XCL1 (lymphotactin), XCL2 (SCM-Ib), XCRI (GPR5/CCXCR1), YY1, ZFPM2, CLEC4C (BDCA-2, DLEC, CD303, CDH6, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), CLEC7A (Dectin-1), CLEC11A, PDGFRa, SLAMF7, GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), LILRB1, NCR1 (CD335, LY94, NKp46), NCR3 (CD335, LY94, NKp46), NCR3 (CD337, NKp30), OSCAR, TARM1, CD30, CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC5, SIGLEC6, SIGLEC7, SIGLEC8, SIGLEC9, SIGLEC10, SIGLEC11, SIGLEC12, SIGLEC14, SIGLEC15 (CD33L3), SIGLEC16, SIRPA, SIRPB1 (CD172B), TREM1 (CD354), TREM2, KLRF1 (NKp80), 17-1A, SLAM7, SLC39A6, MSLN, CTAG1B/NY-ESO-1, MAGEA3/A6, ATP5I (Q06185), OAT (P29758), AIFM1 (Q9Z0X1), AOFA (Q64133), MTDC (P18155), CMC1 (Q8BH59), PREP (Q8K411), YMEL1 (O88967), LPPRC (Q6PB66), LONM (Q8CGK3), ACON (Q99KI0), ODO1 (Q60597), IDHP (P54071), ALDH2 (P47738), ATPB (P56480), AATM (P05202), TMM93 (Q9CQW0), ERGI3 (Q9CQE7), RTN4 (Q99P72), CL041 (Q8BQR4), ERLN2 (Q8BFZ9), TERA (Q01853), DAD1 (P61804), CALX (P35564), CALU (O35887), VAPA (Q9WV55), MOGS (Q80UM7), GANAB (Q8BHN3), ERO1A (Q8R180), UGGG1 (Q6P5E4), P4HA1 (Q60715), HYEP (Q9D379), CALR (P14211), AT2A2 (O55143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (O55222), GPNMB (Q99P91), ENV1 (P10404), ERO1A (Q8R180), CLH (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), and CH10 (Q64433). In the preceding list, synonyms and accession numbers are shown in parentheses.

In some embodiments, the antibody is selected from the group consisting of an anti-PD-L1 antibody, an anti-HER2 antibody, an anti-EGFR antibody, and an anti-CEA antibody. The antibody can be a commercially available antibody, a biosimilar thereof, or a biobetter thereof.

An embodiment of the invention utilizes an antibody construct or antigen binding domain which specifically recognizes and binds to PD-L1 (SEQ ID NO: 1). The antibody construct or antigen binding domain may comprise one or more variable regions (e.g., two variable regions) of an antigen binding domain of an anti-PD-L1 antibody, each variable region comprising a CDR1, a CDR2, and a CDR3.

An embodiment of the invention utilizes an antibody construct or antigen binding domain comprising the CDR regions of atezolizumab. In this regard, the antibody construct or antigen binding domain may comprise a first variable region comprising a CDR1 comprising the amino acid sequence of SEQ ID NO: 2 (CDR1 of first variable region), a CDR2 comprising the amino acid sequence of SEQ ID NO: 3 (CDR2 of first variable region), and a CDR3 comprising the amino acid sequence of SEQ ID NO: 4 (CDR3 of first variable region), and a second variable region comprising a CDR1 comprising the amino acid sequence of SEQ ID NO: 5 (CDR1 of second variable region), a CDR2 comprising the amino acid sequence of SEQ ID NO: 6 (CDR2 of second variable region), and a CDR3 comprising the amino acid sequence of SEQ ID NO: 7 (CDR3 of second variable region). In this regard, the antibody construct can comprise (i) all of SEQ ID NOs: 2-4, (ii) all of SEQ ID NOs: 5-7, or (iii) all of SEQ ID NOs: 2-7. Preferably, the antibody construct or antigen binding domain comprises all of SEQ ID NOs: 2-7.

In an embodiment of the invention, the antibody construct or antigen binding domain comprising the CDR regions of atezolizumab further comprises the framework regions of atezolizumab. In this regard, the antibody construct or antigen binding domain comprising the CDR regions of atezolizumab further comprises the amino acid sequence of SEQ ID NO: 8 (framework region (“FR”) 1 of first variable region), the amino acid sequence of SEQ ID NO: 9 (FR2 of first variable region), the amino acid sequence of SEQ ID NO: 10 (FR3 of first variable region), the amino acid sequence of SEQ ID NO: 11 (FR4 of first variable region), the amino acid sequence of SEQ ID NO: 12 (FR1 of second variable region), the amino acid sequence of SEQ ID NO: 13 (FR2 of second variable region), the amino acid sequence of SEQ ID NO: 14 (FR3 of second variable region), and the amino acid sequence of SEQ ID NO: 15 (FR4 of second variable region). In this regard, the antibody construct or antigen binding domain can comprise (i) all of SEQ ID NOs: 2-4 and 8-11, (ii) all of SEQ ID NOs: 5-7 and 12-15; or (iii) all of SEQ ID NOs: 2-7 and 8-15.

An embodiment of the invention utilizes an antibody construct or antigen binding domain comprising one or both variable regions of atezolizumab. In this regard, the first variable region may comprise SEQ ID NO: 44. The second variable region may comprise SEQ ID NO: 45. Accordingly, in an embodiment of the invention, the antibody construct or antigen binding domain comprises SEQ ID NO: 44, SEQ ID NO: 45, or both SEQ ID NOs: 44 and 45. Preferably, the polypeptide comprises both of SEQ ID NOs: 44-45.

An embodiment of the invention utilizes an antibody construct or antigen binding domain comprising the CDR regions of durvalumab. In this regard, the antibody construct or antigen binding domain may comprise a first variable region comprising a CDR1 comprising the amino acid sequence of SEQ ID NO: 18 (CDR1 of first variable region), a CDR2 comprising the amino acid sequence of SEQ ID NO: 19 (CDR2 of first variable region), and a CDR3 comprising the amino acid sequence of SEQ ID NO: 20 (CDR3 of first variable region), and a second variable region comprising a CDR1 comprising the amino acid sequence of SEQ ID NO: 21 (CDR1 of second variable region), a CDR2 comprising the amino acid sequence of SEQ ID NO: 22 (CDR2 of second variable region), and a CDR3 comprising the amino acid sequence of SEQ ID NO: 23 (CDR3 of second variable region). In this regard, the antibody construct can comprise (i) all of SEQ ID NOs: 18-20, (ii) all of SEQ ID NOs: 21-23, or (iii) all of SEQ ID NOs: 18-23. Preferably, the antibody construct or antigen binding domain comprises all of SEQ ID NOs: 18-23.

In an embodiment of the invention, the antibody construct or antigen binding domain comprising the CDR regions of durvalumab further comprises the framework regions of durvalumab. In this regard, the antibody construct or antigen binding domain comprising the CDR regions of durvalumab further comprises the amino acid sequence of SEQ ID NO: 24 (framework region (“FR”) 1 of first variable region), the amino acid sequence of SEQ ID NO: 25 (FR2 of first variable region), the amino acid sequence of SEQ ID NO: 26 (FR3 of first variable region), the amino acid sequence of SEQ ID NO: 27 (FR4 of first variable region), the amino acid sequence of SEQ ID NO: 28 (FR1 of second variable region), the amino acid sequence of SEQ ID NO: 29 (FR2 of second variable region), the amino acid sequence of SEQ ID NO: 30 (FR3 of second variable region), and the amino acid sequence of SEQ ID NO: 31 (FR4 of second variable region). In this regard, the antibody construct or antigen binding domain can comprise (i) all of SEQ ID NOs: 18-20 and 24-26, (ii) all of SEQ ID NOs: 21-23 and 27-31; or (iii) all of SEQ ID NOs: 18-21 and 24-31.

An embodiment of the invention utilizes an antibody construct or antigen binding domain comprising one or both variable regions of durvalumab. In this regard, the first variable region may comprise SEQ ID NO: 46. The second variable region may comprise SEQ ID NO: 47. Accordingly, in an embodiment of the invention, the antibody construct or antigen binding domain comprises SEQ ID NO: 46, SEQ ID NO: 47, or both SEQ ID NOs: 46 and 47. Preferably, the polypeptide comprises both of SEQ ID NOs: 46-47.

An embodiment of the invention utilizes an antibody construct or antigen binding domain comprising the CDR regions of avelumab. In this regard, the antibody construct or antigen binding domain may comprise a first variable region comprising a CDR1 comprising the amino acid sequence of SEQ ID NO: 30 (CDR1 of first variable region), a CDR2 comprising the amino acid sequence of SEQ ID NO: 31 (CDR2 of first variable region), and a CDR3 comprising the amino acid sequence of SEQ ID NO: 32 (CDR3 of first variable region), and a second variable region comprising a CDR1 comprising the amino acid sequence of SEQ ID NO: 33 (CDR1 of second variable region), a CDR2 comprising the amino acid sequence of SEQ ID NO: 34 (CDR2 of second variable region), and a CDR3 comprising the amino acid sequence of SEQ ID NO: 35 (CDR3 of second variable region). In this regard, the antibody construct can comprise (i) all of SEQ ID NOs: 30-32, (ii) all of SEQ ID NOs: 33-35, or (iii) all of SEQ ID NOs: 30-35. Preferably, the antibody construct or antigen binding domain comprises all of SEQ ID NOs: 30-35.

In an embodiment of the invention, the antibody construct or antigen binding domain comprising the CDR regions of avelumab further comprises the framework regions of avelumab. In this regard, the antibody construct or antigen binding domain comprising the CDR regions of avelumab further comprises the amino acid sequence of SEQ ID NO: 36 (framework region (“FR”) 1 of first variable region), the amino acid sequence of SEQ ID NO: 37 (FR2 of first variable region), the amino acid sequence of SEQ ID NO: 38 (FR3 of first variable region), the amino acid sequence of SEQ ID NO: 39 (FR4 of first variable region), the amino acid sequence of SEQ ID NO: 40 (FR1 of second variable region), the amino acid sequence of SEQ ID NO: 41 (FR2 of second variable region), the amino acid sequence of SEQ ID NO: 42 (FR3 of second variable region), and the amino acid sequence of SEQ ID NO: 43 (FR4 of second variable region). In this regard, the antibody construct or antigen binding domain can comprise (i) all of SEQ ID NOs: 30-32 and 36-39, (ii) all of SEQ ID NOs: 33-35 and 40-43; or (iii) all of SEQ ID NOs: 30-35 and 36-43.

An embodiment of the invention utilizes an antibody construct or antigen binding domain comprising one or both variable regions of avelumab. In this regard, the first variable region may comprise SEQ ID NO: 48. The second variable region may comprise SEQ ID NO: 49. Accordingly, in an embodiment of the invention, the antibody construct or antigen binding domain comprises SEQ ID NO: 48, SEQ ID NO: 49, or both SEQ ID NOs: 48 and 49. Preferably, the polypeptide comprises both of SEQ ID NOs: 48-49.

An embodiment of the invention utilizes antibody construct or antigen binding domain, which specifically recognizes and binds to HER2 (SEQ ID NO: 50). The antibody construct or antigen binding domain may comprise one or more variable regions (e.g., two variable regions) of an antigen binding domain of an anti-HER2 antibody, each variable region comprising a CDR1, a CDR2, and a CDR3.

An embodiment of the invention utilizes an antibody construct or antigen binding domain comprising the CDR regions of trastuzumab. In this regard, the antibody construct or antigen binding domain may comprise a first variable region comprising a CDR1 comprising the amino acid sequence of SEQ ID NO: 51 (CDR1 of first variable region), a CDR2 comprising the amino acid sequence of SEQ ID NO: 52 (CDR2 of first variable region), and a CDR3 comprising the amino acid sequence of SEQ ID NO: 53 (CDR3 of first variable region), and a second variable region comprising a CDR1 comprising the amino acid sequence of SEQ ID NO: 54 (CDR1 of second variable region), a CDR2 comprising the amino acid sequence of SEQ ID NO: 55 (CDR2 of second variable region), and a CDR3 comprising the amino acid sequence of SEQ ID NO: 56 (CDR3 of second variable region). In this regard, the antibody construct can comprise (i) all of SEQ ID NOs: 51-53, (ii) all of SEQ ID NOs: 54-56, or (iii) all of SEQ ID NOs: 51-56. Preferably, the antibody construct or antigen binding domain comprises all of SEQ ID NOs: 51-56.

In an embodiment of the invention, the antibody construct or antigen binding domain comprising the CDR regions of trastuzumab further comprises the framework regions of trastuzumab. In this regard, the antibody construct or antigen binding domain comprising the CDR regions of trastuzumab further comprises the amino acid sequence of SEQ ID NO: 57 (framework region (“FR”) 1 of first variable region), the amino acid sequence of SEQ ID NO: 58 (FR2 of first variable region), the amino acid sequence of SEQ ID NO: 59 (FR3 of first variable region), the amino acid sequence of SEQ ID NO: 60 (FR4 of first variable region), the amino acid sequence of SEQ ID NO: 61 (FR1 of second variable region), the amino acid sequence of SEQ ID NO: 62 (FR2 of second variable region), the amino acid sequence of SEQ ID NO: 63 (FR3 of second variable region), and the amino acid sequence of SEQ ID NO: 64 (FR4 of second variable region). In this regard, the antibody construct or antigen binding domain can comprise (i) all of SEQ ID NOs: 51-53 and 57-60, (ii) all of SEQ ID NOs: 54-56 and 61-64; or (iii) all of SEQ ID NOs: 57-59 and 65-68.

An embodiment of the invention utilizes an antibody construct or antigen binding domain comprising one or both variable regions of trastuzumab. In this regard, the first variable region may comprise SEQ ID NO: 65. The second variable region may comprise SEQ ID NO: 66. Accordingly, in an embodiment of the invention, the antibody construct or antigen binding domain comprises SEQ ID NO: 65, SEQ ID NO: 66, or both SEQ ID NOs: 65 and 66. Preferably, the polypeptide comprises both of SEQ ID NOs: 65-66.

An embodiment of the invention utilizes an antibody construct or antigen binding domain comprising the CDR regions of pertuzumab. In this regard, the antibody construct or antigen binding domain may comprise a first variable region comprising a CDR1 comprising the amino acid sequence of SEQ ID NO: 67 (CDR1 of first variable region), a CDR2 comprising the amino acid sequence of SEQ ID NO: 68 (CDR2 of first variable region), and a CDR3 comprising the amino acid sequence of SEQ ID NO: 69 (CDR3 of first variable region), and a second variable region comprising a CDR1 comprising the amino acid sequence of SEQ ID NO: 70 (CDR1 of second variable region), a CDR2 comprising the amino acid sequence of SEQ ID NO: 71 (CDR2 of second variable region), and a CDR3 comprising the amino acid sequence of SEQ ID NO: 72 (CDR3 of second variable region). In this regard, the antibody construct can comprise (i) all of SEQ ID NOs: 67-69, (ii) all of SEQ ID NOs: 70-72, or (iii) all of SEQ ID NOs: 67-72. Preferably, the antibody construct or antigen binding domain comprises all of SEQ ID NOs: 67-72.

In an embodiment of the invention, the antibody construct or antigen binding domain comprising the CDR regions of pertuzumab further comprises the framework regions of pertuzumab. In this regard, the antibody construct or antigen binding domain comprising the CDR regions of pertuzumab further comprises the amino acid sequence of SEQ ID NO: 73 (framework region (“FR”) 1 of first variable region), the amino acid sequence of SEQ ID NO: 74 (FR2 of first variable region), the amino acid sequence of SEQ ID NO: 75 (FR3 of first variable region), the amino acid sequence of SEQ ID NO: 76 (FR4 of first variable region), the amino acid sequence of SEQ ID NO: 77 (FR1 of second variable region), the amino acid sequence of SEQ ID NO: 78 (FR2 of second variable region), the amino acid sequence of SEQ ID NO: 79 (FR3 of second variable region), and the amino acid sequence of SEQ ID NO: 80 (FR4 of second variable region). In this regard, the antibody construct or antigen binding domain can comprise (i) all of SEQ ID NOs: 67-69 and 73-76, (ii) all of SEQ ID NOs: 70-72 and 77-80; or (iii) all of SEQ ID NOs: 67-72 and 73-80.

An embodiment of the invention utilizes an antibody construct or antigen binding domain comprising one or both variable regions of pertuzumab. In this regard, the first variable region may comprise SEQ ID NO: 81. The second variable region may comprise SEQ ID NO: 82. Accordingly, in an embodiment of the invention, the antibody construct or antigen binding domain comprises SEQ ID NO: 81, SEQ ID NO: 82, or both SEQ ID NOs: 81 and 82. Preferably, the polypeptide comprises both of SEQ ID NOs: 81-82.

An embodiment of the invention provides antibody construct or antigen binding domain which specifically recognizes and binds to CEA (SEQ ID NO: 83). The antibody construct or antigen binding domain may comprise one or more variable regions (e.g., two variable regions) of an antigen binding domain of an anti-CEA antibody, each variable region comprising a CDR1, a CDR2, and a CDR3.

An embodiment of the invention utilizes an antibody construct or antigen binding domain comprising the CDR regions of labetuzumab. In this regard, the antibody construct or antigen binding domain may comprise a first variable region comprising a CDR1 comprising the amino acid sequence of SEQ ID NO: 84 (CDR1 of first variable region), a CDR2 comprising the amino acid sequence of SEQ ID NO: 85 (CDR2 of first variable region), and a CDR3 comprising the amino acid sequence of SEQ ID NO: 86 (CDR3 of first variable region), and a second variable region comprising a CDR1 comprising the amino acid sequence of SEQ ID NO: 87 (CDR1 of second variable region), a CDR2 comprising the amino acid sequence of SEQ ID NO: 88 (CDR2 of second variable region), and a CDR3 comprising the amino acid sequence of SEQ ID NO: 89 (CDR3 of second variable region). In this regard, the antibody construct can comprise (i) all of SEQ ID NOs: 84-86, (ii) all of SEQ ID NOs: 87-89, or (iii) all of SEQ ID NOs: 84-89. Preferably, the antibody construct or antigen binding domain comprises all of SEQ ID NOs: 84-89.

In an embodiment of the invention, the antibody construct or antigen binding domain comprising the CDR regions of labetuzumab further comprises the framework regions of labetuzumab. In this regard, the antibody construct or antigen binding domain comprising the CDR regions of labetuzumab further comprises the amino acid sequence of SEQ ID NO: 90 (framework region (“FR”) 1 of first variable region), the amino acid sequence of SEQ ID NO: 91 (FR2 of first variable region), the amino acid sequence of SEQ ID NO: 92 (FR3 of first variable region), the amino acid sequence of SEQ ID NO: 93 (FR4 of first variable region), the amino acid sequence of SEQ ID NO: 94 (FR1 of second variable region), the amino acid sequence of SEQ ID NO: 95 (FR2 of second variable region), the amino acid sequence of SEQ ID NO: 96 (FR3 of second variable region), and the amino acid sequence of SEQ ID NO: 97 (FR4 of second variable region). In this regard, the antibody construct or antigen binding domain can comprise (i) all of SEQ ID NOs: 84-86 and 90-93, (ii) all of SEQ ID NOs: 87-89 and 94-97; or (iii) all of SEQ ID NOs: 84-89 and 90-97.

An embodiment of the invention utilizes an antibody construct or antigen binding domain comprising one or both variable regions of labetuzumab. In this regard, the first variable region may comprise SEQ ID NO: 98. The second variable region may comprise SEQ ID NO: 99. Accordingly, in an embodiment of the invention, the antibody construct or antigen binding domain comprises SEQ ID NO: 98, SEQ ID NO: 99, or both SEQ ID NOs: 98 and 99. Preferably, the polypeptide comprises both of SEQ ID NOs: 98-99.

An embodiment of the invention utilizes an antibody construct or antigen binding domain comprising the CDR regions of PR1A3. In this regard, the antibody construct or antigen binding domain may comprise a first variable region comprising a CDR1 comprising the amino acid sequence of SEQ ID NO: 100 (CDR1 of first variable region), a CDR2 comprising the amino acid sequence of SEQ ID NO: 101 (CDR2 of first variable region), and a CDR3 comprising the amino acid sequence of SEQ ID NO: 102 (CDR3 of first variable region), and a second variable region comprising a CDR1 comprising the amino acid sequence of SEQ ID NO: 103 (CDR1 of second variable region), a CDR2 comprising the amino acid sequence of SEQ ID NO: 104 (CDR2 of second variable region), and a CDR3 comprising the amino acid sequence of SEQ ID NO: 105 (CDR3 of second variable region). In this regard, the antibody construct can comprise (i) all of SEQ ID NOs: 100-102, (ii) all of SEQ ID NOs: 103-105, or (iii) all of SEQ ID NOs: 100-105. Preferably, the antibody construct or antigen binding domain comprises all of SEQ ID NOs: 100-105.

In an embodiment of the invention, the antibody construct or antigen binding domain comprising the CDR regions of PR1A3 further comprises the framework regions of PR1A3. In this regard, the antibody construct or antigen binding domain comprising the CDR regions of PR1A3 further comprises the amino acid sequence of SEQ ID NO: 106 (framework region (“FR”) 1 of first variable region), the amino acid sequence of SEQ ID NO: 107 (FR2 of first variable region), the amino acid sequence of SEQ ID NO: 108 (FR3 of first variable region), the amino acid sequence of SEQ ID NO: 109 (FR4 of first variable region), the amino acid sequence of SEQ ID NO: 110 (FR1 of second variable region), the amino acid sequence of SEQ ID NO: 111 (FR2 of second variable region), the amino acid sequence of SEQ ID NO: 112 (FR3 of second variable region), and the amino acid sequence of SEQ ID NO: 113 (FR4 of second variable region). In this regard, the antibody construct or antigen binding domain can comprise (i) all of SEQ ID NOs: 100-102 and 106-109, (ii) all of SEQ ID NOs: 103-105 and 110-113; or (iii) all of SEQ ID NOs: 100-103 and 106-113.

An embodiment of the invention utilizes an antibody construct or antigen binding domain comprising one or both variable regions of PR1A3. In this regard, the first variable region may comprise SEQ ID NO: 114. The second variable region may comprise SEQ ID NO: 115. Accordingly, in an embodiment of the invention, the antibody construct or antigen binding domain comprises SEQ ID NO: 114, SEQ ID NO: 115, or both SEQ ID NOs: 114 and 115. Preferably, the polypeptide comprises both of SEQ ID NOs: 114-115.

An embodiment of the invention utilizes an antibody construct or antigen binding domain comprising the CDR regions of MFE-23. In this regard, the antibody construct or antigen binding domain may comprise a first variable region comprising a CDR1 comprising the amino acid sequence of SEQ ID NO: 116 (CDR1 of first variable region), a CDR2 comprising the amino acid sequence of SEQ ID NO: 117 (CDR2 of first variable region), and a CDR3 comprising the amino acid sequence of SEQ ID NO: 118 (CDR3 of first variable region), and a second variable region comprising a CDR1 comprising the amino acid sequence of SEQ ID NO: 119 (CDR1 of second variable region), a CDR2 comprising the amino acid sequence of SEQ ID NO: 120 (CDR2 of second variable region), and a CDR3 comprising the amino acid sequence of SEQ ID NO: 121 (CDR3 of second variable region). In this regard, the antibody construct can comprise (i) all of SEQ ID NOs: 116-118, (ii) all of SEQ ID NOs: 119-121, or (iii) all of SEQ ID NOs: 116-121. Preferably, the antibody construct or antigen binding domain comprises all of SEQ ID NOs: 116-121.

In an embodiment of the invention, the antibody construct or antigen binding domain comprising the CDR regions of MFE-23 further comprises the framework regions of MFE-23. In this regard, the antibody construct or antigen binding domain comprising the CDR regions of MFE-23 further comprises the amino acid sequence of SEQ ID NO: 122 (framework region (“FR”) 1 of first variable region), the amino acid sequence of SEQ ID NO: 123 (FR2 of first variable region), the amino acid sequence of SEQ ID NO: 124 (FR3 of first variable region), the amino acid sequence of SEQ ID NO: 125 (FR4 of first variable region), the amino acid sequence of SEQ ID NO: 126 (FR1 of second variable region), the amino acid sequence of SEQ ID NO: 127 (FR2 of second variable region), the amino acid sequence of SEQ ID NO: 128 (FR3 of second variable region), and the amino acid sequence of SEQ ID NO: 129 (FR4 of second variable region). In this regard, the antibody construct or antigen binding domain can comprise (i) all of SEQ ID NOs: 116-118 and 122-125, (ii) all of SEQ ID NOs: 119-121 and 126-129; or (iii) all of SEQ ID NOs: 116-121 and 122-129.

An embodiment of the invention utilizes an antibody construct or antigen binding domain comprising one or both variable regions of MFE-23. In this regard, the first variable region may comprise SEQ ID NO: 130. The second variable region may comprise SEQ ID NO: 131. Accordingly, in an embodiment of the invention, the antibody construct or antigen binding domain comprises SEQ ID NO: 130, SEQ ID NO: 131, or both SEQ ID NOs: 130 and 131. Preferably, the polypeptide comprises both of SEQ ID NOs: 130-131.

An embodiment of the invention utilizes an antibody construct or antigen binding domain comprising the CDR regions of SM3E. In this regard, the antibody construct or antigen binding domain may comprise a first variable region comprising a CDR1 comprising the amino acid sequence of SEQ ID NO: 132 (CDR1 of first variable region), a CDR2 comprising the amino acid sequence of SEQ ID NO: 133 (CDR2 of first variable region), and a CDR3 comprising the amino acid sequence of SEQ ID NO: 134 (CDR3 of first variable region), and a second variable region comprising a CDR1 comprising the amino acid sequence of SEQ ID NO: 135 (CDR1 of second variable region), a CDR2 comprising the amino acid sequence of SEQ ID NO: 136 (CDR2 of second variable region), and a CDR3 comprising the amino acid sequence of SEQ ID NO: 137 (CDR3 of second variable region). In this regard, the antibody construct can comprise (i) all of SEQ ID NOs: 132-134, (ii) all of SEQ ID NOs: 135-137, or (iii) all of SEQ ID NOs: 132-137. Preferably, the antibody construct or antigen binding domain comprises all of SEQ ID NOs: 132-137.

In an embodiment of the invention, the antibody construct or antigen binding domain comprising the CDR regions of SM3E further comprises the framework regions of SM3E. In this regard, the antibody construct or antigen binding domain comprising the CDR regions of SM3E further comprises the amino acid sequence of SEQ ID NO: 138 (framework region (“FR”) 1 of first variable region), the amino acid sequence of SEQ ID NO: 139 (FR2 of first variable region), the amino acid sequence of SEQ ID NO: 140 (FR3 of first variable region), the amino acid sequence of SEQ ID NO: 141 (FR4 of first variable region), the amino acid sequence of SEQ ID NO: 142 (FR1 of second variable region), the amino acid sequence of SEQ ID NO: 143 (FR2 of second variable region), the amino acid sequence of SEQ ID NO: 144 (FR3 of second variable region), and the amino acid sequence of SEQ ID NO: 145 (FR4 of second variable region). In this regard, the antibody construct or antigen binding domain can comprise (i) all of SEQ ID NOs: 132-134 and 138-53, (ii) all of SEQ ID NOs: 135-137 and 142-144; or (iii) all of SEQ ID NOs: 132-137 and 138-144.

An embodiment of the invention utilizes an antibody construct or antigen binding domain comprising one or both variable regions of SM3E. In this regard, the first variable region may comprise SEQ ID NO: 146. The second variable region may comprise SEQ ID NO: 147. Accordingly, in an embodiment of the invention, the antibody construct or antigen binding domain comprises SEQ ID NO: 146, SEQ ID NO: 147, or both SEQ ID NOs: 146 and 147. Preferably, the polypeptide comprises both of SEQ ID NOs: 146-147.

An embodiment of the invention utilizes an antibody construct or antigen binding domain comprising the CDR regions of the anti-EGFR antibody cetuximab. In this regard, the antibody construct or antigen binding domain may comprise a first variable region comprising a CDR1 comprising the amino acid sequence of SEQ ID NO: 148 (CDR1 of first variable region), a CDR2 comprising the amino acid sequence of SEQ ID NO: 149 (CDR2 of first variable region), and a CDR3 comprising the amino acid sequence of SEQ ID NO: 150 (CDR3 of first variable region), and a second variable region comprising a CDR1 comprising the amino acid sequence of SEQ ID NO: 151 (CDR1 of second variable region), a CDR2 comprising the amino acid sequence of SEQ ID NO: 152 (CDR2 of second variable region), and a CDR3 comprising the amino acid sequence of SEQ ID NO: 153 (CDR3 of second variable region). In this regard, the antibody construct or antigen binding domain can comprise (i) all of SEQ ID NOs: 148-150, (ii) all of SEQ ID NOs: 151-153, or (iii) all of SEQ ID NOs: 148-153. Preferably, the antibody construct or antigen binding domain comprises all of SEQ ID NOs: 148-153.

An embodiment of the invention utilizes an antibody construct or antigen binding domain comprising the CDR regions of the anti-EGFR antibody panitumumab. In this regard, the antibody may comprise a first variable region comprising a CDR1 comprising the amino acid sequence of SEQ ID NO: 154 (CDR1 of first variable region), a CDR2 comprising the amino acid sequence of SEQ ID NO: 155 (CDR2 of first variable region), and a CDR3 comprising the amino acid sequence of SEQ ID NO: 156 (CDR3 of first variable region), and a second variable region comprising a CDR1 comprising the amino acid sequence of SEQ ID NO: 157 (CDR1 of second variable region), a CDR2 comprising the amino acid sequence of SEQ ID NO: 158 (CDR2 of second variable region), and a CDR3 comprising the amino acid sequence of SEQ ID NO: 159 (CDR3 of second variable region). In this regard, antibody construct or antigen binding domain can comprise (i) all of SEQ ID NOs: 154-156, (ii) all of SEQ ID NOs: 157-159, or (iii) all of SEQ ID NOs: 154-159. Preferably, antibody construct or antigen binding domain comprises all of SEQ ID NOs: 154-159.

An embodiment of the invention utilizes antibody construct or antigen binding domain comprising the CDR regions of the anti-EGFR antibody necitumumab. In this regard, the antibody construct or antigen binding domain may comprise a first variable region comprising a CDR1 comprising the amino acid sequence of SEQ ID NO: 160 (CDR1 of first variable region), a CDR2 comprising the amino acid sequence of SEQ ID NO: 161 (CDR2 of first variable region), and a CDR3 comprising the amino acid sequence of SEQ ID NO: 162 (CDR3 of first variable region), and a second variable region comprising a CDR1 comprising the amino acid sequence of SEQ ID NO: 163 (CDR1 of second variable region), a CDR2 comprising the amino acid sequence of SEQ ID NO: 164 (CDR2 of second variable region), and a CDR3 comprising the amino acid sequence of SEQ ID NO: 165 (CDR3 of second variable region). In this regard, antibody construct or antigen binding domain can comprise (i) all of SEQ ID NOs: 160-162, (ii) all of SEQ ID NOs: 163-165, or (iii) all of SEQ ID NOs: 160-165. Preferably, antibody construct or antigen binding domain comprises all of SEQ ID NOs: 160-165.

An embodiment of the invention utilizes an antibody construct or antigen binding domain comprising one or both variable regions of the anti-EGFR antibody cetuximab. In this regard, the first variable region may comprise SEQ ID NO: 166. The second variable region may comprise SEQ ID NO: 167. Accordingly, in an embodiment of the invention, the antibody comprises SEQ ID NO: 166, SEQ ID NO: 167, or both SEQ ID NOs: 166 and 167. Preferably, the antibody comprises both of SEQ ID NOs: 166-167.

In addition to antibodies, alternative protein scaffolds may be used as part of the immunoconjugates. For example, an alternative protein scaffold may replace the antibody construct of Formula II such that the therapeutic agent/linker is bound to a lysine residue of the alternative protein scaffold. The phrase “alternative protein scaffold” refers to a non-immunoglobulin derived protein or peptide. Such proteins and peptides are generally amenable to engineering and can be designed to confer monospecificity against a given antigen, bispecificity, or multispecificity. Engineering of an alternative protein scaffold can be conducted using several approaches. A loop grafting approach can be used where sequences of known specificity are grafted onto a variable loop of a scaffold. Sequence randomization and mutagenesis can be used to develop a library of mutants, which can be screened using various display platforms (e.g., phage display) to identify a novel binder. Site-specific mutagenesis can also be used as part of a similar approach. Alternative protein scaffolds exist in a variety of sizes, ranging from small peptides with minimal secondary structure to large proteins of similar size to a full sized antibody. Examples of scaffolds include, but are not limited to, cystine knotted miniproteins (also known as knottins), cyclic cystine knotted miniproteins (also known as cyclotides), avimers, affibodies, the tenth type III domain of human fibronectin, DARPins (designed ankyrin repeats), and anticalins (also known as lipocalins). Naturally occurring ligands with known specificity can also be engineered to confer novel specificity against a given target. Examples of naturally occurring ligands that may be engineered include the EGF ligand and VEGF ligand. Engineered proteins can either be produced as monomeric proteins or as multimers, depending on the desired binding strategy and specificities. Protein engineering strategies can be used to fuse alternative protein scaffolds to Fc domains.

In some embodiments, the antibody construct binds to an FcRγ-coupled receptor. In some embodiments, the FcRγ-coupled receptor is selected from the group consisting of GP6 (GPVI), LILRA1 (CD851), LILRA2 (CD85H, ILT1), LILRA4 (CD85G, ILT7), LILRA5 (CD85F, IL T11), LILRA6 (CD85b, ILT8), LILRB1, NCR1 (CD335, LY94, NKp46), NCR3 (CD335, LY94, NKp46), NCR3 (CD337, NKp30), OSCAR, and TARM1.

In some embodiments, the antibody construct binds to a DAP12-coupled receptor. In some embodiments, the DAP12-coupled receptor is selected from the group consisting of CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC5, SIGLEC6, SIGLEC7, SIGLEC8, SIGLEC9, SIGLEC10, SIGLEC11, SIGLEC12, SIGLEC14, SIGLEC15 (CD33L3), SIGLEC16, SIRPB1 (CD172B), TREM1 (CD354), and TREM2.

In some embodiments, the antibody construct binds to a hemITAM-bearing receptor. In some embodiments, the hemITAM-bearing receptor is KLRF1 (NKp80).

In some embodiments, the antibody is capable of binding one or more targets selected from CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), and CLEC7A (Dectin-1). In some embodiments, the antibody is capable of binding CLEC6A (Dectin-2) or CLEC5A. In some embodiments, the antibody is capable of binding CLEC6A (Dectin-2).

In some embodiments, the antibody construct is capable of binding one or more targets selected from (e.g., specifically binds to a target selected from): ATP5I (Q06185), OAT (P29758), AIFM1 (Q9Z0X1), AOFA (Q64133), MTDC (P18155), CMC1 (Q8BH59), PREP (Q8K411), YMEL1 (O88967), LPPRC (Q6PB66), LONM (Q8CGK3), ACON (Q99KI0), ODO1 (Q60597), IDHP (P54071), ALDH2 (P47738), ATPB (P56480), AATM (P05202), TMM93 (Q9CQW0), ERGI3 (Q9CQE7), RTN4 (Q99P72), CL041 (Q8BQR4), ERLN2 (Q8BFZ9), TERA (Q01853), DAD1 (P61804), CALX (P35564), CALU (O35887), VAPA (Q9WV55), MOGS (Q80UM7), GANAB (Q8BHN3), ERO1A (Q8R180), UGGG1 (Q6P5E4), P4HA1 (Q60715), HYEP (Q9D379), CALR (P14211), AT2A2 (O55143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (O55222), GPNMB (Q99P91), ENV1 (P10404), ERO1A (Q8R180), CLH (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), and CH10 (Q64433). In the preceding list, accession numbers are shown in parentheses.

In some embodiments, the antibody construct binds to an antigen selected from CCR8, CDH1, CD19, CD20, CD29, CD30, CD38, CD40, CD47, EpCAM, MUC1, MUC16, EGFR, HER2, SLAMF7, and gp75. In some embodiments, the antigen is selected from CCR8, CD19, CD20, CD47, EpCAM, MUC1, MUC16, EGFR, and HER2. In some embodiments, the antibody construct binds to an antigen selected from the Tn antigen and the Thomsen-Friedenreich antigen.

In some embodiments, the antibody construct is selected from: abagovomab, abatacept (also known as ORENCIA™), abciximab (also known as REOPRO™, c7E3 Fab), adalimumab (also known as HUMIRA™), adecatumumab, alemtuzumab (also known as CAMPATH™, MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (also known as SIMULECT™), bavituximab, bectumomab (also known as LYMPHOSCAN™), belimumab (also known as LYMPHO-STAT-B™), bertilimumab, besilesomab, bevacizumab (also known as AVASTIN™) biciromab brallobarbital, bivatuzumab mertansine, campath, canakinumab (also known as ACZ885), cantuzumab mertansine, capromab (also known as PROSTASCINT™) catumaxomab (also known as REMOVAB™), cedelizumab (also known as CIMZIA™), certolizumab pegol, cetuximab (also known as ERBITUX™), clenoliximab, dacetuzumab, dacliximab, daclizumab (also known as ZENAPAX™), denosumab (also known as AMG 162), detumomab, dorlimomab aritox, dorlixizumab, duntumumab, durimulumab, durmulumab, ecromeximab, eculizumab (also known as SOLIRIS™), edobacomab, edrecolomab (also known as Mab17-1A, PANOREX™), efalizumab (also known as RAPTIVA™), efungumab (also known as MYCOGRAB™), elotuzumab, elsilimomab, enlimomab pegol, epitumomab cituxetan, efalizumab, epitumomab, epratuzumab, erlizumab, ertumaxomab (also known as REXOMUN™), etanercept (also known as ENBREL™) etaracizumab (also known as etaratuzumab, VITAXIN™, ABEGRIN™), exbivirumab, fanolesomab (also known as NEUTROSPEC™), faralimomab, felvizumab, fontolizumab (also known as HUZAF™), galiximab, gantenerumab, gavilimomab (also known as ABXCBL™), gemtuzumab ozogamicin (also known as MYLOTARG™), golimumab (also known as CNTO 148), gomiliximab, ibalizumab (also known as TNX-355), ibritumomab tiuxetan (also known as ZEVALIN™), igovomab, imeiromab, infliximab (also known as REMICADE™) inolimomab, inotuzumab ozogamicin, ipilimumab (also known as MDX-010, MDX-101), iratumumab, keliximab, labetuzumab, lemalesomab, lebrilizumab, lerdelimumab, lexatumumab (also known as, HGS-ETR2, ETR2-ST01), lexitumumab, libivirumab, lintuzumab, lucatumumab, lumiliximab, mapatumumab (also known as HGSETR1, TRM-1), maslimomab, matuzumab (also known as EMD72000), mepolizumab (also known as BOSATRIA™), metelimumab, milatuzumab, minretumomab, mitumomab, morolimumab, motavizumab (also known as NUMAX™), muromonab (also known as OKT3), nacolomab tafenatox, naptumomab estafenatox, natalizumab (also known as TYSABRI™ ANTEGREN™), nebacumab, nerelimomab, nimotuzumab (also known as THERACIM hR3™, THERA-CIM-hR3™, THERALOC™), nofetumomab merpentan (also known as VERLUMA™), obinutuzumab, ocrelizumab, odulimomab, ofatumumab, omalizumab (also known as XOLAIR™), oregovomab (also known as OVAREX™), otelixizumab, pagibaximab, palivizumab (also known as SYNAGIS™), panitumumab (also known as ABX-EGF, VECTIBIX™), pascolizumab, pemtumomab (also known as THERAGYN™) pertuzumab (also known as 2C4, OMNITARG™), pexelizumab, pintumomab, priliximab, pritumumab, ranibizumab (also known as LUCENTIS™), raxibacumab, regavirumab, reslizumab, rituximab (also known as RITUXAN™, MabTHERA™), rovelizumab, ruplizumab, satumomab, sevirumab, sibrotuzumab, siplizumab (also known as MEDI-507), sontuzumab, stamulumab (also known as MYO-029), sulesomab (also known as LEUKOSCAN™), tacatuzumab tetraxetan, tadocizumab, talizumab, taplitumomab paptox, tefibazumab (also known as AUREXIS™), telimomab aritox, teneliximab, teplizumab, ticilimumab, tocilizumab (also known as ACTEMRA™), toralizumab, tositumomab, trastuzumab (also known as HERCEPTIN™), tremelimumab (also known as CP-675,206), tucotuzumab celmoleukin, tuvirumab, urtoxazumab, ustekinumab (also known as CNTO 1275), vapaliximab, veltuzumab, vepalimomab, visilizumab (also known as NUVION™), volociximab (also known as M200), votumumab (also known as HUMASPECT™) zalutumumab, zanolimumab (also known as HuMAX-CD4), ziralimumab, zolimomab aritox, daratumumab, olaratumab, brentuximab vedotin, afibercept, abatacept, belatacept, afibercept, etanercept, romiplostim, SBT-040 (sequences listed in U.S. 2017/0158772. In some embodiments, the antibody construct is selected from the group consisting of olaratumab, obinutuzumab, trastuzumab, cetuximab, rituximab, pertuzumab, bevacizumab, daratumumab, etanercept, pembrolizumab, nivolumab, atezolizumab, ipilimumab, panitumumab, zalutumumab, nimotuzumab, matuzumab, and elotuzumab. In certain embodiments, the antibody construct is trastuzumab.

Checkpoint Inhibitors

Any suitable immune checkpoint inhibitor is contemplated for co-administration with the immunoconjugates disclosed herein. In some embodiments, the immune checkpoint inhibitor reduces the expression or activity of one or more immune checkpoint proteins. In another embodiment, the immune checkpoint inhibitor reduces the interaction between one or more immune checkpoint proteins and their ligands. Inhibitory nucleic acids that decrease the expression and/or activity of immune checkpoint molecules can also be used in the methods disclosed herein.

Most checkpoint inhibitors are designed not to have effector function as the use of checkpoint inhibitors is not intended to kill cells, but rather to block the signaling. Immunoconjugates of the invention can add back the “effector functionality” needed to activate myeloid immunity. Hence, for most checkpoint inhibitors, this discovery will be critical.

In some embodiments, the immune checkpoint inhibitor is cytotoxic T-lymphocyte antigen 4 (CTLA4, also known as CD152), T cell immunoreceptor with Ig and ITIM domains (TIGIT), glucocorticoid-induced TNFR-related protein (GITR, also known as TNFRSF18), inducible T cell costimulatory (ICOS, also known as CD278), CD96, poliovirus receptor-related 2 (PVRL2, also known as CD112R, programmed cell death protein 1 (PD-1, also known as CD279), programmed cell death 1 ligand 1 (PD-L1, also known as B7-H3 and CD274), programmed cell death ligand 2 (PD-L2, also known as B7-DC and CD273), lymphocyte activation gene-3 (LAG-3, also known as CD223), B7-H4, killer immunoglobulin receptor (KIR), Tumor Necrosis Factor Receptor superfamily member 4 (TNFRSF4, also known as OX40 and CD134) and its ligand OX40L (CD252), indoleamine 2,3-dioxygenase 1 (IDO-1), indoleamine 2,3-dioxygenase 2 (IDO-2), carcinoembryonic antigen-related cell adhesion molecule 1 (CEACAM1), B and T lymphocyte attenuator (BTLA, also known as CD272), T-cell membrane protein 3 (TIM3), the adenosine A2A receptor (A2Ar), and V-domain Ig suppressor of T cell activation (VISTA protein). In some embodiments, the immune checkpoint inhibitor is an inhibitor of CTLA4, PD-1, or PD-L1.

In some embodiments, the immune checkpoint inhibitor is selected from ipilimumab (also known as YERVOY™ pembrolizumab (also known as KEYTRUDA™), nivolumab (also known as OPDIVO™), atezolizumab (also known as TECENTRIG™), avelumab (also known as BAVENCIO™), and durvalumab (also known as IMFINZI™). In some embodiments, the immune checkpoint inhibitor is selected from ipilimumab (also known as YERVOY™), pembrolizumab (also known as KEYTRUDA™), nivolumab (also known as OPDIVO™), and atezolizumab (also known as TECENTRIG™).

Linker

Any suitable linker can be used in the context of the invention provided that that linker can be bound to the antibody construct using an ester described herein.

The linker can have any suitable length such that when the linker is covalently bound to the antibody construct and the therapeutic agent, the function of the antibody construct and the therapeutic agent is maintained. The linker can have a length of about 3 Å or more, for example, about 4 Å or more, about 5 Å or more, about 6 Å or more, about 7 Å or more, about 8 Å or more, about 9 Å or more, about 10 Å or more, or about 20 Å or more. Alternatively, or in addition to, the linker can have a length of about 200 Å or less, for example, about 150 Å or less, about 100 Å or less, about 90 Å or less, about 80 Å or less, about 70 Å or less, about 60 Å or less, about 50 Å or less, about 45 Å or less, about 40 Å or less, about 35 Å or less, about 30 Å or less, about 25 Å or less, about 20 Å or less, or about 15 Å or less. Thus, the linker can have a length bounded by any two of the aforementioned endpoints. The linker can have a length from about 3 Å to about 200 Å, for example, from about 3 Å to about 150 Å, from about 3 Å to about 100 Å, from about 3 Å to about 90 Å, from about 3 Å to about 80 Å, from about 3 Å to about 70 Å, from about 3 Å to about 60 Å, from about 3 Å to about 50 Å, from about 3 Å to about 45 Å, from about 3 Å to about 40 Å, from about 3 Å to about 35 Å, from about 3 Å to about 30 Å, from about 3 Å to about 25 Å, from about 3 Å to about 20 Å, from about 3 Å to about 15 Å, from about 5 Å to about 50 Å, from about 5 Å to about 25 Å, from about 5 Å to about 20 Å, from about 10 Å to about 50 Å, from about 10 Å to about 20 Å, from about 5 Å to about 30 Å, from about 5 Å to about 15 Å, from about 20 Å to about 100 Å, from about 20 Å to about 90 Å, from about 20 Å to about 80 Å, from about 20 Å to about 70 Å, from about 20 Å to about 60 Å, or from about 20 Å to about 50 Å. In certain embodiments, the linker has a length from about 20 Å to about 100 Å.

In some embodiments, the linker is non-cleavable under physiological conditions. As used herein, the term “physiological conditions” refers to a temperature range of 20-40 degrees Celsius, atmospheric pressure (i.e., 1 atm), a pH of about 6 to about 8, and the one or more physiological enzymes, proteases, acids, and bases.

In some embodiments, the linker is cleavable under physiological conditions. For example, the linker can be cleaved by an enzymatic process or a metabolic process.

In some embodiments, the linker comprises a poly(ethylene glycol) group. In certain embodiments, the linker comprises at least 2 ethylene glycol groups (e.g., at least 3 ethylene glycol groups, at least 4 ethylene glycol groups, at least 5 ethylene glycol groups, at least 6 ethylene glycol groups, at least 7 ethylene glycol groups, at least 8 ethylene glycol groups, at least 9 ethylene glycol groups, at least 10 ethylene glycol groups, at least 11 ethylene glycol groups, at least 12 ethylene glycol groups, at least 13 ethylene glycol groups, at least 14 ethylene glycol groups, at least 15 ethylene glycol groups, at least 16 ethylene glycol groups, at least 17 ethylene glycol groups, at least 18 ethylene glycol groups, at least 19 ethylene glycol groups, at least 20 ethylene glycol groups, at least 21 ethylene glycol groups, at least 22 ethylene glycol groups, at least 23 ethylene glycol groups, at least 24 ethylene glycol groups, or at least 25 ethylene glycol groups. In certain embodiments, the linker comprises a di(ethylene glycol) group, a tri(ethylene glycol) group, or a tetra(ethylene glycol) group, 5 ethylene glycol groups, 6 ethylene glycol groups, 8 ethylene glycol groups, 10 ethylene glycol groups, 12 ethylene glycol groups, 24 ethylene glycol groups, or 25 ethylene glycol groups.

In some embodiments, the linker (L) is of the formula:

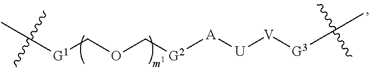

where

A is optionally present and is NR¹ or of formula:

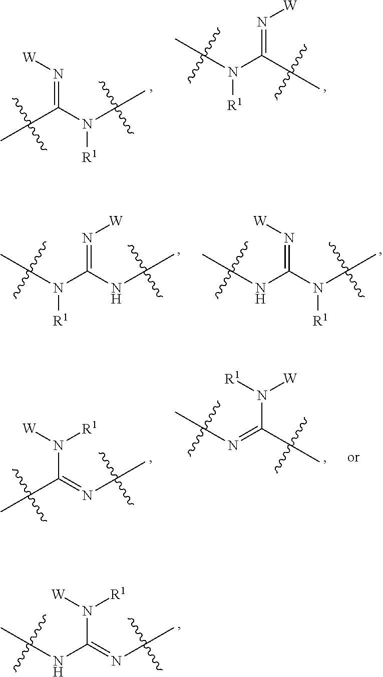

U is optionally present and is CH₂, C(O), CH₂C(O), or C(O)CH₂,

R¹ and W independently are hydrogen, Ar, or of formula:

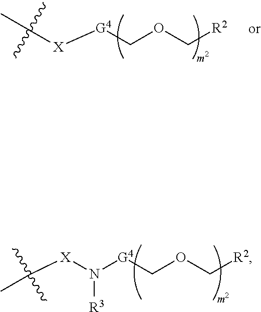

V is optionally present and is of formula:

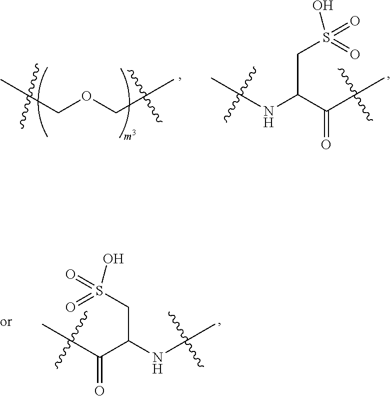

m¹, m², and m³ independently are an integer from 0 to 25, except that at least one of m¹, m², and m³ is a non-zero integer,

G¹, G², G³, and G⁴ independently are CH₂, C(O), CH₂C(O), C(O)CH₂, or a bond,

X is optionally present and is O, NR⁴, CHR⁴, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R², R³, and R⁴ independently are hydrogen or C₁-C₄ alkyl,

Ar is an aryl or heteroaryl group, optionally substituted with one or more halogens (e.g., fluorine, chlorine, bromine, or iodine), nitriles, hydroxyls, C₁-C₄ alkyl groups, or a combination thereof, and

each wavy line (“ [symbol] ”) represents a point of attachment.

In certain embodiments, the linker (L) is of the formula:

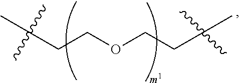

where

m¹ is an integer from 1 to 25 and

each wavy line (“ [symbol] ”) represents a point of attachment.

In certain embodiments, the linker (L) is of the formula:

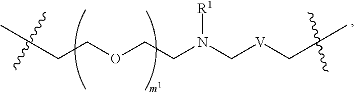

where

R¹ is hydrogen, Ar, or of formula:

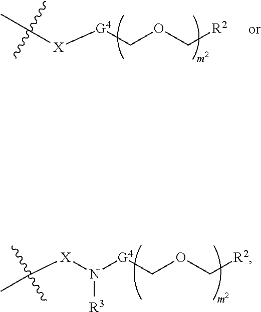

V is optionally present and is of formula:

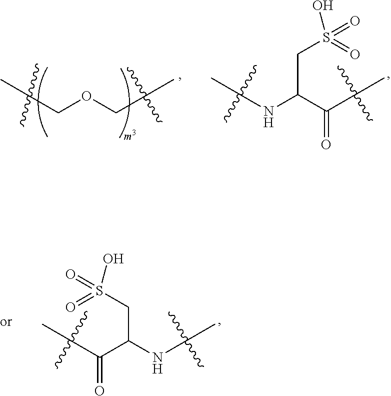

m¹, m², and m³ independently are an integer from 0 to 25, except that at least one of m¹, m², and m³ is a non-zero integer,

G⁴ is CH₂, C(O), CH₂C(O), C(O)CH₂, or a bond,

X is optionally present and is O, NR⁴, CHR⁴, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R², R³, and R⁴ independently are hydrogen or C₁-C₄ alkyl,

Ar is an aryl or heteroaryl group, optionally substituted with one or more halogens (e.g., fluorine, chlorine, bromine, or iodine), nitriles, hydroxyls, C₁-C₄ alkyl groups, or a combination thereof, and

each wavy line (“ [symbol] ”) represents a point of attachment.

In certain embodiments, the linker (L) is of the formula:

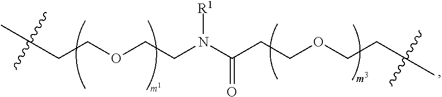

where

U is optionally present and is CH₂, C(O), CH₂C(O), or C(O)CH₂,

R¹ is hydrogen, Ar, or of formula:

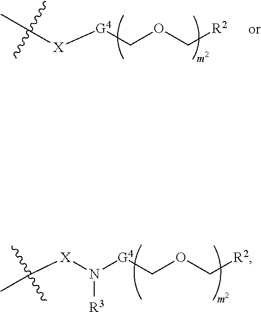

m¹, m², and m³ independently are an integer from 0 to 25, except that at least one of m¹, m², and m³ is a non-zero integer,

G⁴ is CH₂, C(O), CH₂C(O), C(O)CH₂, or a bond,

X is optionally present and is O, NR⁴, CHR⁴, S02, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R², R³, and R⁴ independently are hydrogen or C₁-C₄ alkyl,

Ar is an aryl or heteroaryl group, optionally substituted with one or more halogens (e.g., fluorine, chlorine, bromine, or iodine), nitriles, hydroxyls, C₁-C₄ alkyl groups, or a combination thereof, and

each wavy line (“ [symbol] ”) represents a point of attachment.

In certain embodiments, the linker (L) is of the formula:

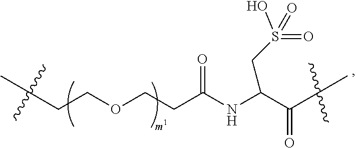

where

m¹ is an integer from 1 to 25 and

each wavy line (“ [symbol] ”) represents a point of attachment.

In certain embodiments, the linker (L) is of the formula:

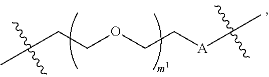

where

A is optionally present and is NR¹ or of formula:

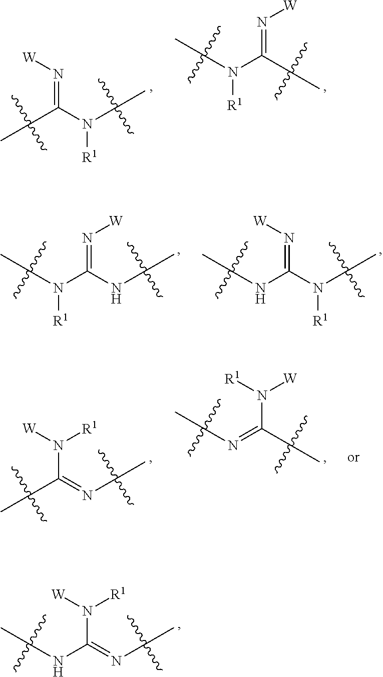

U is optionally present and is CH₂, C(O), CH₂C(O), or C(O)CH₂,

R¹ and W independently are hydrogen, Ar, or of formula:

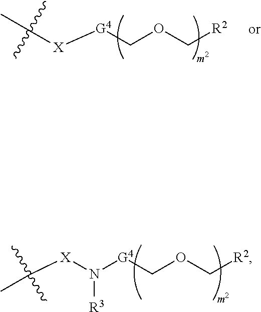

m¹ and m² independently are an integer from 0 to 25, except that at least one of m¹, m², and m³ is a non-zero integer,

G⁴ is CH₂, C(O), CH₂C(O), C(O)CH₂, or a bond,

X is optionally present and is O, NR⁴, CHR⁴, S02, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R², R³, and R⁴ independently are hydrogen or C₁-C₄ alkyl,

Ar is an aryl or heteroaryl group, optionally substituted with one or more halogens (e.g., fluorine, chlorine, bromine, or iodine), nitriles, hydroxyls, C₁-C₄ alkyl groups, or a combination thereof, and

each wavy line (“ [symbol] ”) represents a point of attachment.

In some embodiments, X is one or more divalent groups selected from benzene, naphthalene, pyrrole, indole, isoindole, indolizine, furan, benzofuran, benzothiophene, thiophene, pyridine, acridine, naphthyridine, quinolone, isoquinoline, isoxazole, oxazole, benzoxazole, isothiazole, thiazole, benzthiazole, imidazole, thiadiazole, tetrazole, triazole, oxadiazole, benzimidazole, purine, pyrazole, pyrazine, pteridine, quinoxaline, phthalazine, quinazoline, triazine, phenazine, cinnoline, pyrimidine, pyridazine, cyclohexane, decahydronaphthalene, pyrrolidine, octahydroindole, octahydroisoindole, tetrahydrofuran, octahydrobenzofuran, octahydrobenzothiophene, tetrahydrothiophene, piperidine, tetradecahydroacridine, naphthyridine, decahydroquinoline, decahydroisoquinoline, isoxazolidine, oxazolidine, octahydrobenzooxazole, isothiazolidine, thiazolidine, octahydrobenzothiazole, imidazolidine, 1,2,3-thiadiazolidine, tetrazolidine, 1,2,3-triazolidine, 1,2,3-oxadiazolidine, octahydrobenzoimidazole, octahydropurine, pyrazolidine, piperazine, dechydropteridine, decahydroquinoxaline, dechydrophthalazine, dechydroquinazoline, 1,3,5-triazinane, tetradecahydrophenazine, decahydrocinnoline, hexhydropyrimidine, or hexahydropyridazine. In some embodiments, the one or more divalent groups of X are fused. In some embodiments, the one or more divalent groups of X are linked through a bond or —CO—. In certain embodiments, X can be substituted with one or more halogens (e.g., fluorine, chlorine, bromine, or iodine), nitriles, hydroxyls, C₁-C₄ alkyl groups, or a combination thereof.

In certain embodiments, X is of formula:

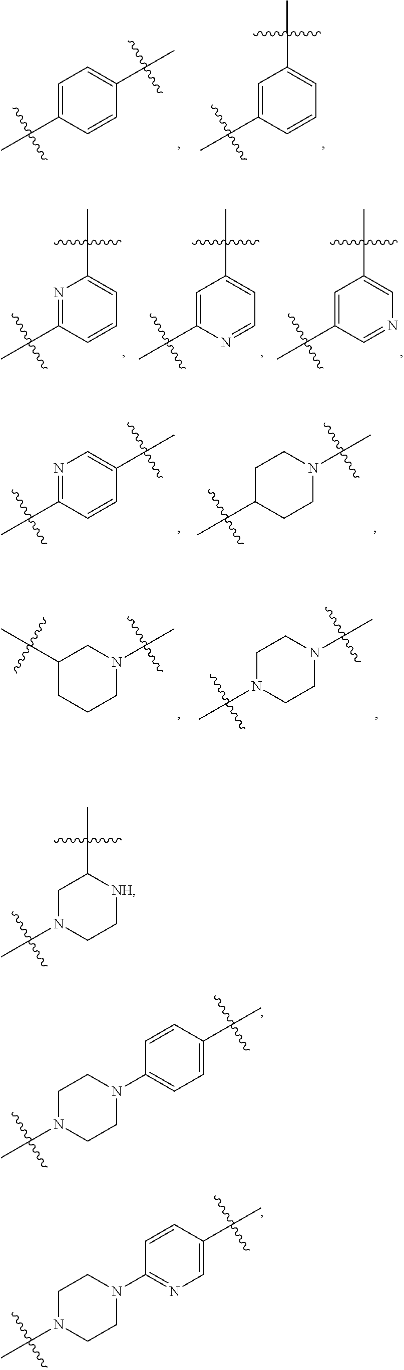

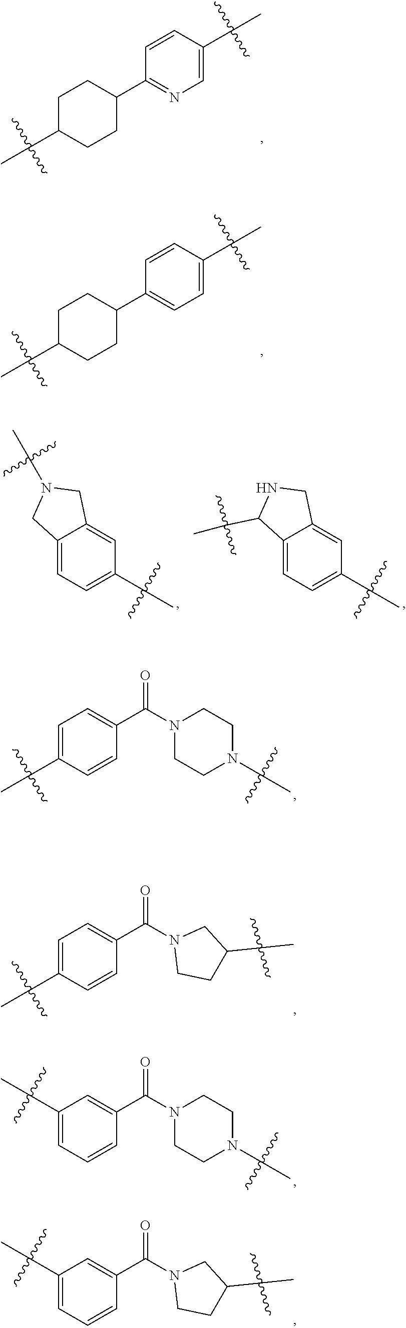

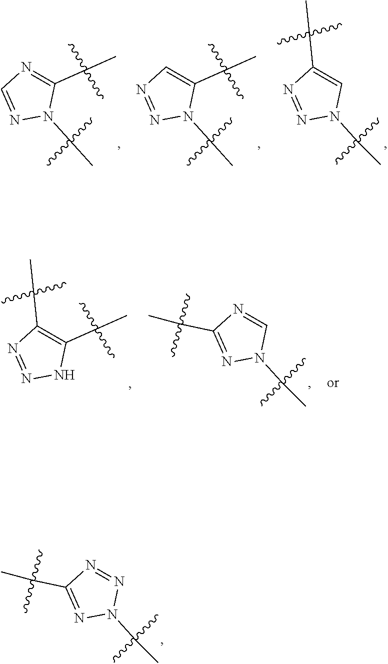 [SMILES: *c1ncnn1* ; *c1cnnn1* ; *c1cn(*)nn1 ; *c1nn[nH]c1* ; *c1ncn(*)n1 ; *c1nnn(*)n1]

wherein any of the above-referenced structures can be used bilaterally.

Ar is an aryl or heteroaryl group, optionally substituted with one or more halogens (e.g., fluorine, chlorine, bromine, or iodine), nitriles, hydroxyls, C₁-C₄ alkyl groups, or a combination thereof. Ar can be any suitable aryl or heteroaryl group described herein. In some embodiments, Ar is a monovalent aryl or heteroaryl group described by the divalent groups of X, optionally substituted with one or more halogens (e.g., fluorine, chlorine, bromine, or iodine), nitriles, hydroxyls, C₁-C₄ alkyl groups, or a combination thereof.

Variables m¹, m², and m³ independently are an integer from 0 to 25. Typically, at least one of m¹, m², and m³ is a non-zero integer such that at least one of m¹, m², and m³ is an integer from 1 to 25. In certain embodiments, at least one of m¹, m², and m³ is an integer from about 2 to about 25 (e.g., about 2 to about 16, about 6 to about 25, about 6 to about 16, about 8 to about 25, about 8 to about 16, about 6 to about 12, or about 8 to about 12). Accordingly, in some embodiments, the immunoconjugates of the invention comprise about 2 to about 25 (e.g., about 2 to about 16, about 6 to about 25, about 6 to about 16, about 8 to about 25, about 8 to about 16, about 6 to about 12, or about 8 to about 12) ethylene glycol units, as designated with subscripts “m¹”, “m²” and “m³”. Accordingly, the immunoconjugates of the invention can comprise at least 2 ethylene glycol groups (e.g., at least 3 ethylene glycol groups, at least 4 ethylene glycol groups, at least 5 ethylene glycol groups, at least 6 ethylene glycol groups, at least 7 ethylene glycol groups, at least 8 ethylene glycol groups, at least 9 ethylene glycol groups, or at least 10 ethylene glycol groups). Accordingly, the immunoconjugate can comprise from about 2 to about 25 ethylene glycol units, for example, from about 6 to about 25 ethylene glycol units, from about 6 to about 16 ethylene glycol units, from about 8 to about 25 ethylene glycol units, from about 8 to about 16 ethylene glycol units, from about 8 to about 12 ethylene glycol units, or from about 8 to about 12 ethylene glycol units. In certain embodiments, the immunoconjugate comprises a di(ethylene glycol) group, a tri(ethylene glycol) group, a tetra(ethylene glycol) group, 5 ethylene glycol groups, 6 ethylene glycol groups, 7 ethylene glycol groups, 8 ethylene glycol groups, 9 ethylene glycol groups, 10 ethylene glycol groups, 11 ethylene glycol groups, 12 ethylene glycol groups, 13 ethylene glycol groups, 14 ethylene glycol groups, 15 ethylene glycol groups, 16 ethylene glycol groups, 24 ethylene glycol groups, or 25 ethylene glycol groups.

In some embodiments, the linker (L) is selected from the group consisting of:
-(PEP)-C(═O)-(PEG)-C(═O)—;
—NR⁵-(PEG)-C(═O)—;
-(PEP)-C(═O)-(PEG)-NR⁵-(PEG)-C(═O)—;
-(PEP)-C(═O)-(PEG)-N⁺(R⁵)₂-(PEG)-C(═O)—;
—C(═O)-(PEG)-C(═O)—;
—C(═O)—CH(AA₁)-NR⁵—C(═O)-(PEG)-C(═O)—;
-(PEP)-C(═O)-(PEG)-C(═O)—CH(AA₁)-NR⁵-(PEG)-C(═O)—;
—C(═O)O—(C₁-C₁₂ alkyldiyl)-S—S-(PEG)-C(═O)—;
—C(═O)—(C₁-C₁₂ alkyldiyl)-S—S-(PEG)-C(═O)—;
-(PEG)-C(═O)—;
-(PEP)-C(═O)—(C₁-C₁₂ alkyldiyl)-C(═O)—;
-(MCgluc)-(C(═O)-(PEG)-OCH₂—(C₁-C₂₀ heteroaryldiyl)-CH₂CH₂OCH₂CH₂—C(═O)—;
-(PEP)-C(═O)—(CH₂)ₘ—C(═O)—; and
-(PEP)-C(═O)—(CH₂)ₘ—;

where

PEG has the formula:

—(CH₂CH₂O)ₙ—(CH₂)ₘ—; m is an integer from 1 to 5, and n is an integer from 2 to 50;

PEP has the formula:

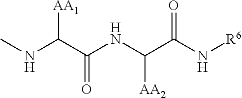

where AA₁ and AA₂ are independently selected from an amino acid side chain; and R⁶ is selected from the group consisting of C₆-C₂₀ aryldiyl and C₁-C₂₀ heteroaryldiyl, substituted with —CH₂O—C(═O)— and optionally with:

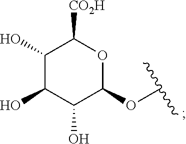 [SMILES: *O[C@@H]1O[C@H](C(=O)O)[C@@H](O)[C@H](O)[C@H]1O]

MCgluc has the formula:

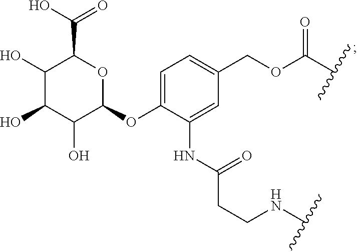 [SMILES: *NCCC(=O)Nc1cc(COC(*)=O)ccc1O[C@@H]1O[C@H](C(=O)O)C(O)[C@H](O)C1O]

and

where alkyl, alkyldiyl, aryl, aryldiyl carbocyclyl, carbocyclyldiyl, heterocyclyl, heterocyclyldiyl, heteroaryl, and heteroaryldiyl are optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OP(O)(OH)₂, —S(O)₂N(CH₃)₂, —SCH₃, —S(O)₂CH₃, and —S(O)₃H.

Each of the linkers described herein can be used bilaterally unless otherwise specified. However, in certain embodiments, the linkers described herein are interpreted as read from left to right on the page.

### Therapeutic Agents

In some embodiments, the therapeutic agent is a compound that elicits an immune response (e.g., an immune agonist or antagonist). In certain embodiments, the therapeutic agent is a pattern recognition receptor (“PRR”) agonist. Any therapeutic agent capable of activating a pattern recognition receptor (PRR) can be installed in the immunoconjugates of the invention. As used herein, the terms “Pattern recognition receptor” and “PRR” refer to any member of a class of conserved mammalian proteins, which recognize pathogen-associated molecular patterns (“PAMPs”) or damage-associated molecular patterns (“DAMPs”), and act as key signaling elements in innate immunity. Pattern recognition receptors are divided into membrane-bound PRRs, cytoplasmic PRRs, and secreted PRRs. Examples of membrane-bound PRRs include Toll-like receptors (“TLRs”) and C-type lectin receptors (“CLRs”). Examples of cytoplasmic PRRs include NOD-like receptors (NLRs), such as NLRP3, Rig-I-like receptors (RLR), and STING (STimulator of INterferon Genes). In some embodiments, the immunoconjugate can have more than one distinct PRR therapeutic agent.

In some immunoconjugates of the invention, the therapeutic agent is a TLR agonist. TLR agonists include TLR1, TLR2, TLR3, TLR4, TLR5, TLR6, TLR7, TLR8, TLR9, TLR10, TLR11, or any combination thereof (e.g., TLR7/8 agonists). Any therapeutic agent capable of activating a TLR can be utilized in the immunoconjugates of the invention. TLRs are type-I transmembrane proteins that are responsible for initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate within vertebrates. Once engaged (e.g., by a natural stimulus or a synthetic TLR agonist) TLRs initiate a signal transduction cascade leading to activation of NF-κB via the adapter protein myeloid differentiation primary response gene 88 (MyD88) and recruitment of the IL-1 receptor associated kinase (IRAK). Phosphorylation of IRAK then leads to recruitment of TNF-receptor associated factor (TRAF) 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TRAF6 and activation of MyD88 independent pathways via TRIF and TRAF3, leading to the phosphorylation of interferon response factor (IRF) three (IRF3). Similarly, the MyD88 dependent pathway also activates several IRF family members, including IRF5 and IRF7 whereas the TRIF dependent pathway also activates the NF-κB pathway.

Examples of TLR2 agonists include but are not limited to an agent comprising N-α-palmitoyl-S-[2,3-bis(palmitoyloxy)-(2RS)-propyl]-L-cysteine, palmitoyl-Cys((RS)-2,3-di(palmitoyloxy)-propyl) (“Pam3Cys”), e.g., Pam3Cys, Pam3Cys-Ser-(Lys)4 (also known as “Pam3Cys-SKKKK” and “Pam₃CSK₄”), Triacyl lipid A (“OM-174”), Lipoteichoic acid (“LTA”), peptidoglycan, and CL419 (S-(2,3-bis(palmitoyloxy)-(2RS)propyl)-(R)-cysteinyl spermine).

An example of a TLR2/6 agonist is Pam₂CSK₄ (S-[2,3-bis(palmitoyloxy)-(2RS)-propyl]-[R]-cysteinyl-[S]-seryl-[S]-lysyl-[S]-lysyl-[S]-lysyl-[S]-lysine x 3 CF3COOH).

Examples of TLR2/7 agonist include CL572 (S-(2-myristoyloxy ethyl)-(R)-cysteinyl 4-((6-amino-2-(butylamino)-8-hydroxy-9H-purin-9-yl)methyl) aniline), CL413 (S-(2,3-bis(palmitoyloxy)-(2RS)propyl)-(R)-cysteinyl-(S)-seryl-(S)-lysyl-(S)-lysyl-(S)-lysyl-(S)-lysyl 4-((6-amino-2-(butylamino)-8-hydroxy-9H-purin-9-yl)methyl)aniline), and CL401 (S-(2,3-bis(palmitoyloxy)-(2RS)propyl)-(R)-cysteinyl 4-((6-amino-2(butyl amino)-8-hydroxy-9H-purin-9-yl)methyl) aniline).

Examples of TLR3 agonists include Polyinosine-polycytidylic acid (poly (I.C)), Polyadenylic-polyuridylic acid (poly (A:U), and poly(I)-poly(C12U).

Examples of TLR4 agonists include Lipopolysaccharide (LPS) and Monophosphoryl lipid A (MPLA).

An example of a TLR5 agonist includes Flagellin.

Examples of TLR9 agonists include single strand CpG oligodeoxynucleotides (CpG ODN). Three major classes of stimulatory CpG ODNs have been identified based on structural characteristics and activity on human peripheral blood mononuclear cells (PBMCs), in particular B cells and plasmacytoid dendritic cells (pDCs). These three classes are Class A (Type D), Class B (Type K) and Class C.

Examples of Nod Like Receptor (NLR) agonists include acylated derivative of iE-DAP, D-gamma-Glu-mDAP, L-Ala-gamma-D-Glu-mDAP, Muramyldipeptide with a C18 fatty acid chain, Muramyldipeptide, muramyl tripeptide, and N-glycolylated muramyldipeptide.

Examples of RIG-I-Like receptor (RLR) agonists include 5′ppp-dsrna (5′-pppGCAUGCGACCUCUGUUUGA-3′(SEQ ID NO: 169): 3′-CGUACGCUGGAGACAAACU-5′ (SEQ ID NO: 170)), and Poly(deoxyadenylic-deoxythymidylic) acid (Poly(dA:dT))

Additional immune-stimulatory compounds, such as cytosolic DNA and unique bacterial nucleic acids called cyclic dinucleotides, can be recognized by STING, which can act a cytosolic DNA sensor. ADU-SlOO can be a STING agonist. Non-limiting examples of STING agonists include: Cyclic [G(2′,5′)pA(2′,5′)p] (2′2′-cGAMP), cyclic [G(2′,5′)pA(3′,5′)p] (2′3′-cGAMP), cyclic [G(3′,5′)pA(3′,5′)p] (3′3′-cGAMP), Cyclic di-adenylate monophosphate (c-di-AMP), 2′,5′-3′,5′-c-diAMP (2′3′-c-di-AMP), Cyclic di-guanylate monophosphate (c-di-GMP), 2′,5′-3′,5′-c-diGMP (2′3′-c-di-GMP), Cyclic di-inosine monophosphate (c-di-IMP), Cyclic di-uridine monophosphate (c-di-UMP), KIN700, KIN1148, KIN600, KIN500, KIN100, KIN101, KIN400, KIN2000, or SB-9200 can be recognized.

In certain embodiments, the therapeutic agent is a TLR7 and/or TLR8 agonist. Any therapeutic agent capable of activating TLR7 and/or TLR8 can be utilized in the immunoconjugates of the invention. Examples of TLR7 agonists and TLR8 agonists are described, for example, by Vacchelli et al. (Oncolmmunology, 2(8): e25238 (2013), which is hereby incorporated by reference in its entirety herein) and Carson et al. (U.S. Patent Application Publication 2013/0165455, which is hereby incorporated by reference in its entirety herein). TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell as a means to respond to viral invasion. Treatment of TLR8-expressing cells with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

The methods described herein are particularly useful for hydrophobic therapeutic agents. Without wishing to be bound by any particular theory, it is believed that hydrophobic therapeutic agents reduce solubility, such that reaction rate, percent yield, and conjugation selectivity may be affected. Thus, the ester moieties provided herein can be particularly useful in counteracting the lack of solubility of the hydrophobic therapeutic agents. As such, in some embodiments, the therapeutic agent (i.e., the therapeutic agent in the absence of the linker) has an octanol/water partition, log(P), of greater than about 1, e.g., greater than about 1.5, greater than about 2, greater than about 2.5, greater than about 3, greater than about 3.5, greater than about 4, greater than about 4.5, or greater than about 5. In certain embodiments, the therapeutic agent (i.e., the therapeutic agent in the absence of the linker) has an octanol/water partition, log(P), of greater than about 3. In preferred embodiments, the therapeutic agent (i.e., the therapeutic agent in the absence of the linker) has an octanol/water partition, log(P), of greater than about 5.

Therapeutic agents particularly useful for the methods and immunoconjugates provided herein are described, for example, in U.S. Patent Application Publication 2019/0015516; International Patent Application Publication WO 2018/112108; and U.S. Provisional Patent Applications 62/861,117, 62/819,365, and 62/724,259; which are hereby incorporated by reference in their entireties.

Formulation and Administration of Immunoconjugates

In a related aspect, the invention provides a composition comprising a plurality of immunoconjugates as described above. In some embodiments, the average number of therapeutic agents per immunoconjugate ranges from about 1 to about 50. The average number of therapeutic agents per immunoconjugate can range, for example, from about 1 to about 10, from about 1 to about 8, or from about 1 to about 6, or from about 1 to about 4. The average number of therapeutic agents per immunoconjugate can be about 0.8, 1, 1.2, 1.4, 1.6, 1.8, 2, 2.2, 2.4, 2.6, 2.8, 3, 3.2, 3.4, 3.6, 3.8, 4.0, or 4.2. In some embodiments, the average number of therapeutic agents per immunoconjugate is about 4. In some embodiments, the average number of therapeutic agents per immunoconjugate is about 2. In some cases, the antibody is covalently bonded to a single therapeutic agent. In some cases, the antibody is covalently bonded to 2 or more therapeutic agents (e.g., 3 or more, 4 or more, or 5 or more therapeutic agents) via a linker. In some cases, the antibody is covalently bonded to 1-8 therapeutic agents (e.g., 1-5, 1-3, 2-8, 2-5, 2-3, or 3-8 therapeutic agents) via a linker. In some cases, the antibody is covalently bonded to 2-8 therapeutic agents (e.g., 2-5, 2-3, or 3-8 therapeutic agents). In some cases in which the antibody is covalently bonded to more than one therapeutic agent, the attached therapeutic agents can be the same or different. For example, in some cases two or more of the therapeutic agents can be the same (e.g., two different molecules of the same therapeutic agent can each be attached to the antibody at a different site on the antibody). In some cases, the antibody is covalently bonded to 2 or more different therapeutic agents (e.g., 3 or more, 4 or more, or 5 or more different therapeutic agents). For example, when generating an immunoconjugate of the invention, one or more antibodies can be combined with a mixture that includes two or more (e.g., 3 or more, 4 or more, or 5 or more) different therapeutic agent-linker compounds such that amino acid sidechains in the one or more antibodies react with the therapeutic agent-linker compounds, thereby resulting in one or more immunoconjugates that are each covalently bonded to two or more different therapeutic agents.

In some embodiments, the composition further comprises one or more pharmaceutically acceptable excipients. For example, the immunoconjugates of the invention can be formulated for parenteral administration, such as intravenous (IV) administration or administration into a body cavity or lumen of an organ. Alternatively, the immunoconjugates can be injected intra-tumorally. Formulations for injection will commonly comprise a solution of the immunoconjugate dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the immunoconjugate in these formulations can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.1% (w/w) to about 10% (w/w).

The invention provides a method for treating and/or preventing cancer. The method includes administering a therapeutically effective amount of an immunoconjugate as described herein (e.g., as a composition as described herein) to a subject in need thereof, e.g., a subject that has cancer and is in need of treatment for the cancer. In some embodiments, the cancer is susceptible to an immune response resulting from a therapeutic agent that is an immune antagonist (e.g., an immune receptor antagonist). In some embodiments, the cancer is susceptible to an immune response resulting from a therapeutic agent that is an immune agonist (e.g., an immune receptor agonist).

The invention provides a method for treating and/or preventing a disease or condition (e.g., autoimmune diseases, viral infections, etc.). The method includes administering a therapeutically effective amount of an immunoconjugate as described herein (e.g., as a composition as described herein) to a subject in need thereof, e.g., a subject that has a disease or condition (e.g., autoimmune diseases, viral infections, etc.) and is in need of treatment for the disease or condition. In some embodiments, the disease or condition is susceptible to an immune response resulting from a therapeutic agent that is an immune antagonist (e.g., an immune receptor antagonist). In some embodiments, the disease or condition is susceptible to an immune response resulting from a therapeutic agent that is an immune agonist (e.g., an immune receptor agonist). It is contemplated that the immunoconjugate of the invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, an immunoconjugate for use as a medicament is provided. In certain embodiments, the invention provides an immunoconjugate for use in a method of treating an individual comprising administering to the individual an effective amount of the immunoconjugate. In one such embodiment, the method further comprises administering to the individual an effective amount of at least one additional therapeutic agent, e.g., as described herein.

In a further aspect, the invention provides for the use of an immunoconjugate in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin. In some embodiments, methods for treating non-small cell lung carcinoma include administering an immunoconjugate containing an antibody construct that is capable of binding PD-L1 (e.g., atezolizumab, durvalumab, avelumab, biosimilars thereof, or bio betters thereof). In some embodiments, methods for treating breast cancer include administering an immunoconjugate containing an antibody construct that is capable of binding PD-L1 (e.g., atezolizumab, durvalumab, avelumab, biosimilars thereof, or biobetters thereof). In some embodiments, methods for treating triple-negative breast cancer include administering an immunoconjugate containing an antibody construct that is capable of binding PD-L1 (e.g., atezolizumab, durvalumab, avelumab, biosimilars thereof, or biobetters thereof).

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck. Merkel cell carcinoma is also called neuroendocrine carcinoma of the skin. In some embodiments, methods for treating Merkel cell carcinoma include administering an immunoconjugate containing an antibody construct that is capable of binding PD-L1 (e.g., atezolizumab, durvalumab, avelumab, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Immunoconjugates of the invention can be used either alone or in combination with other agents in a therapy. For instance, an immunoconjugate may be co-administered with at least one additional drug, such as a chemotherapeutic agent. Such combination therapies encompass combined administration (where two or more drugs or therapeutic agents are included in the same or separate formulations), and separate administration, in which case, administration of the immunoconjugate can occur prior to, simultaneously, and/or following, administration of the additional therapeutic agents and/or drugs. Immunoconjugates can also be used in combination with radiation therapy.

The immunoconjugates of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Atezolizumab, durvalumab, avelumab, biosimilars thereof, and biobetters thereof are known to be useful in the treatment of cancer, particularly breast cancer, especially triple negative (test negative for estrogen receptors, progesterone receptors, and excess HER2 protein) breast cancer, bladder cancer, and Merkel cell carcinoma. The immunoconjugate described herein can be used to treat the same types of cancers as atezolizumab, durvalumab, avelumab, biosimilars thereof, and biobetters thereof, particularly breast cancer, especially triple negative (test negative for estrogen receptors, progesterone receptors, and excess HER2 protein) breast cancer, bladder cancer, and Merkel cell carcinoma.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer. In some embodiments, methods for treating breast cancer include administering an immunoconjugate containing an antibody construct that is capable of binding HER2 (e.g., trastuzumab, pertuzumab, biosimilars thereof, or biobetters thereof).

Some embodiments of the invention provide methods for treating cancer as described above, wherein the cancer is gastric cancer. Gastric (stomach) cancer can originate from different cells in the stomach and several types of gastric cancer have been characterized including adenocarcinoma, carcinoid tumors, squamous cell carcinoma, small cell carcinoma, leiomyosarcoma, and gastrointestinal stromal tumors. In some embodiments, methods for treating gastric cancer include administering an immunoconjugate containing an antibody construct that is capable of binding HER2 (e.g., trastuzumab, pertuzumab, biosimilars thereof, or biobetters thereof).

Some embodiments of the invention provide methods for treating cancer as described above, wherein the cancer is gastroesophageal junction carcinoma. This carcinoma occurs in the area where the esophagus meats the stomach. There are three types of gastroesophageal junction carcinoma. In Type 1, the cancer the cancer grows down from above and into the gastroesophageal junction. The normal lining of the lower end of the esophagus is replaced by mutations (also called Barrett's esophagus). In Type 2, the cancer grows at the gastroesophageal junction by itself. In Type 3, the cancer grows up into the gastroesophageal junction from the stomach upwards. In some embodiments, methods for treating gastroesophageal junction carcinoma include administering an immunoconjugate containing an antibody construct that is capable of binding HER2 (e.g., trastuzumab, pertuzumab, biosimilars thereof, or biobetters thereof).

Some embodiments of the invention provide methods to treat the same types of cancers as labetuzumab, PR1A3, MFE-23, SM3E, biosimilars thereof, and biobetters thereof, particularly colon cancer, lung cancer, renal cancer, pancreatic cancer, gastric cancer, and esophageal cancer, especially CEA-overexpressing colon cancer, lung cancer, renal cancer, pancreatic cancer, gastric cancer, and esophageal cancer. In preferred embodiments, the immunoconjugates described herein can be used to treat colon cancer.

Some embodiments of the invention provide methods for treating cancer as described above, wherein the cancer is a head and neck cancer. Head and neck cancer (as well as head and neck squamous cell carcinoma) refers to a variety of cancers characterized by squamous cell carcinomas of the oral cavity, pharynx and larynx, salivary glands, paranasal sinuses, and nasal cavity, as well as the lymph nodes of the upper part of the neck. Head and neck cancers account for approximately 3 to 5 percent of all cancers in the United States. These cancers are more common in men and in people over age 50. Tobacco (including smokeless tobacco) and alcohol use are the most important risk factors for head and neck cancers, particularly those of the oral cavity, oropharynx, hypopharynx and larynx. Eighty-five percent of head and neck cancers are linked to tobacco use.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer. In some embodiments, methods for treating breast cancer include administering an immunoconjugate containing an antibody that is capable of binding EGFR (e.g., cetuximab).

The immunoconjugate is administered to a subject in need thereof in any therapeutically effective amount using any suitable dosing regimen, such as the dosing regimens utilized for atezolizumab, durvalumab, avelumab, biosimilars thereof, and biobetters thereof. For example, the methods can include administering the immunoconjugate to provide a dose of from about 100 ng/kg to about 50 mg/kg (based on the weight of the subject) to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented. For example, the methods can include administering the immunoconjugate to provide a dose of from about 100 ng/kg to about 50 mg/kg (based on the weight of the subject) to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer. In some embodiments, methods for treating breast cancer include administering an immunoconjugate containing an antibody construct that is capable of binding HER2 (e.g. trastuzumab, pertuzumab, biosimilars, or biobetters thereof) or PD-L1 (e.g., atezolizumab, durvalumab, avelumab, biosimilars, or biobetters thereof).

In some embodiments, the cancer is susceptible to a pro-inflammatory response induced by TLR7 and/or TLR8.

### EMBODIMENTS

Aspects, including embodiments, of the present subject matter described herein may be beneficial alone or in combination, with one or more other aspects or embodiments. Without limiting the foregoing description, certain non-limiting aspects of the disclosure numbered 1-27 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method for producing an immunoconjugate, the method comprising combining one or more compounds of Formula I:

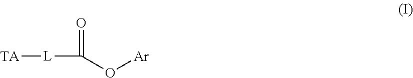

or salts thereof,
and an antibody construct of Formula II:

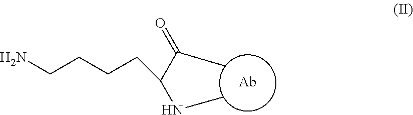

or salt thereof,

wherein Formula II is an antibody construct with residue

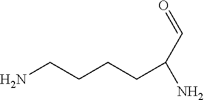

representing one or more lysine residues of the antibody construct,

to provide the immunoconjugate of Formula III:

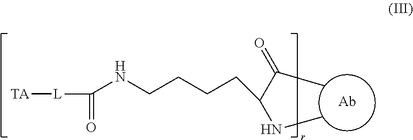

or salt thereof,
wherein

TA is a therapeutic agent,

L is a linker,

r is an integer from 1 to 50,

Ar is an aromatic moiety comprising a first substituent selected from PEG, —SO₂CX₃, —NR₃⁺, —NO₂, —SO₃R, —SO₂R, —CN, —CX₃, —PO₃R₂, —OPO₃R₂,

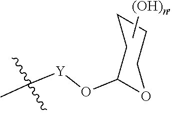

and salts thereof,

each R independently is H, CX₃, or C₁-C₄ alkyl,

each X independently is hydrogen or a halogen,

Y is CH₂, PEG, or a bond,

n is an integer from 1 to 4, and

PEG has the formula:

—(CH₂CH₂O)ₘ—(CH₂)ₚ—,

where p is an integer from 1 to 5 and m is an integer from 2 to 50.

2. The method of aspect 1, wherein Ar further comprises one or more additional substituents selected from —F, —Cl, —Br, —I, —CR₃, —OR, —C(O)R, —C(O)OR, PEG, —SO₂CX₃, —NR₃⁺, —NO₂, —SO₃R, —SO₂R, —CN, —CX₃, —PO₃R₂, —OPO₃R₂,

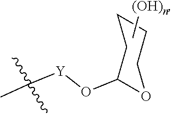

salts thereof, and combinations thereof,

wherein each R independently is H, CX₃, or C₁-C₄ alkyl,

each X independently is hydrogen or a halogen,

Y is CH₂, PEG, or a bond,

n is an integer from 1 to 4, and

PEG has the formula:

—(CH₂CH₂O)ₘ—(CH₂)ₚ—,

where p is an integer from 1 to 5 and m is an integer from 2 to 50.

3. The method of aspect 1 or aspect 2, wherein the first substituent is selected from —NO₂, —SO₃H, —CN, and salts thereof.

4. The method of any one of aspects 1-3, wherein the first substituent is —SO₃H or a salt thereof.

5. The method of any one of aspects 2-4, wherein the one or more additional substituents is selected from —F, —Cl, —Br, —I, —NO₂, —SO₃H, —CN, and salts thereof.

6. The method of any one of aspects 2-5, wherein the one or more additional substituents is selected from —F, —Cl, —Br, and —I.

7. The method of any one of aspects 1-6, wherein Ar is of formula:

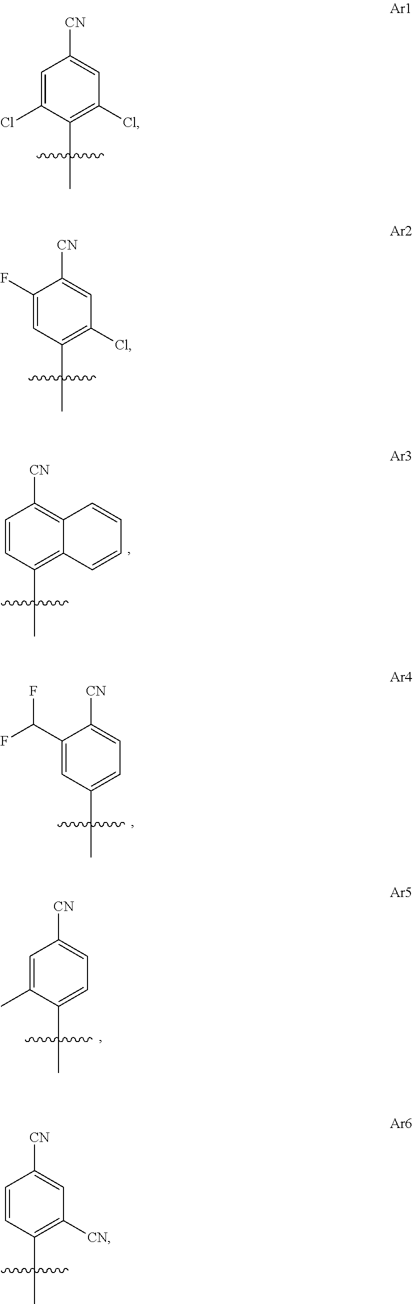

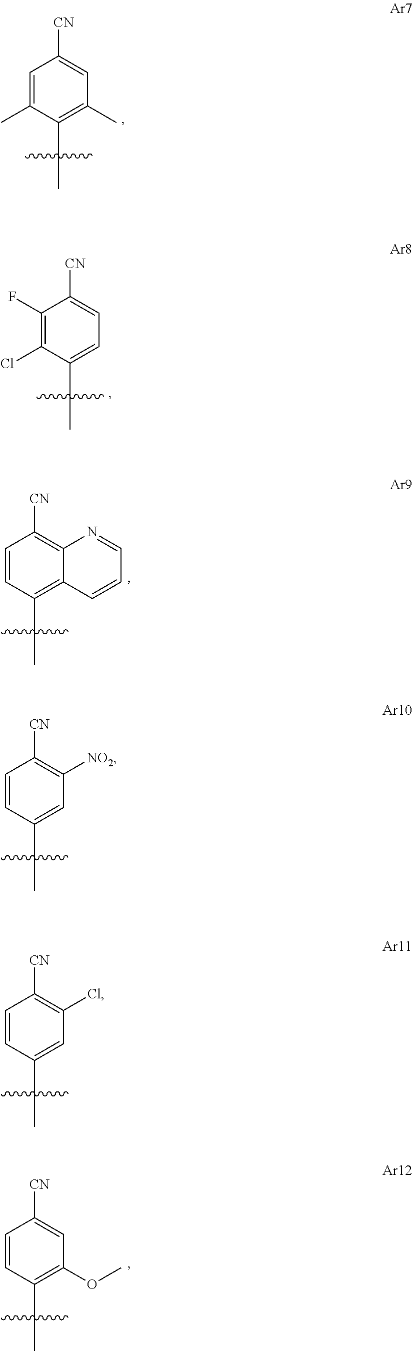

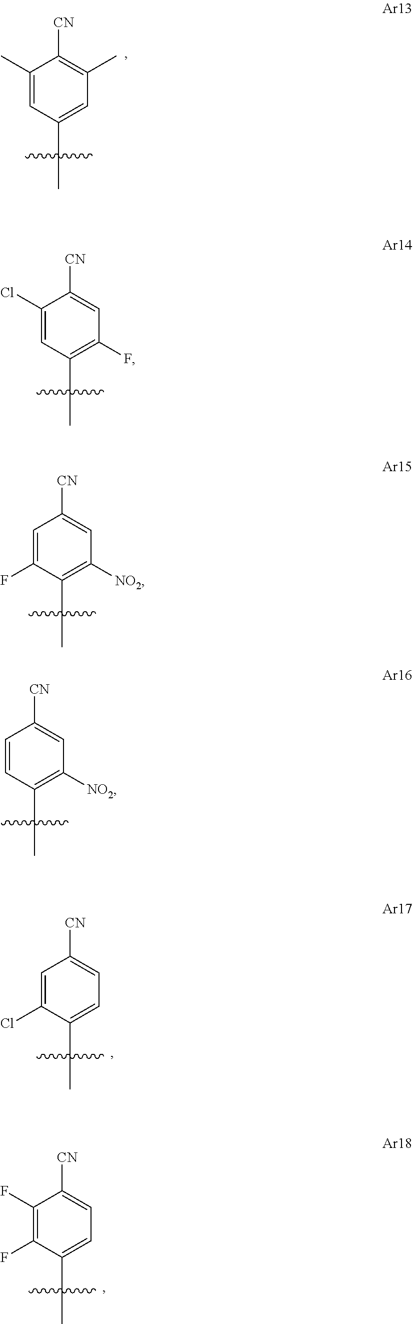

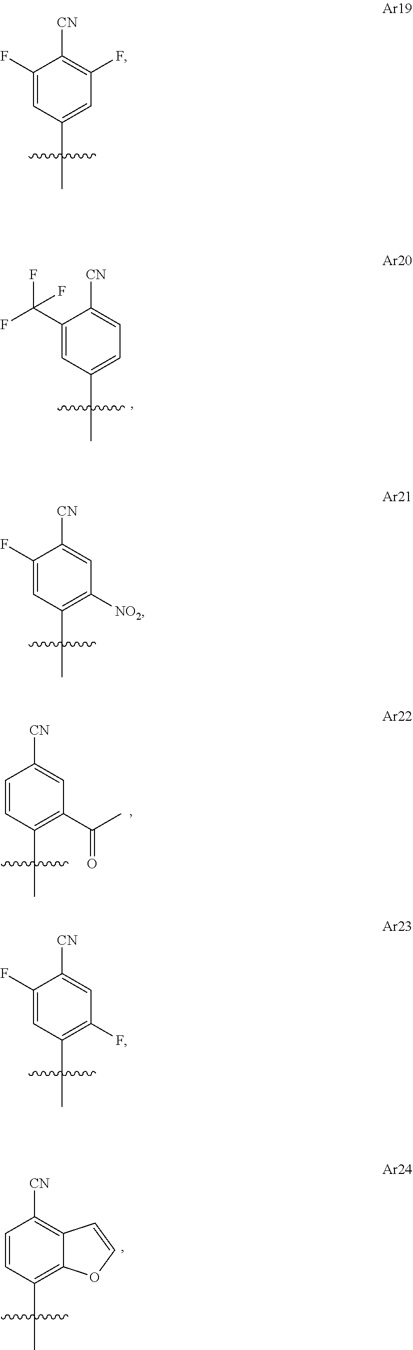 [SMILES: *c1cc(F)c(C#N)c(F)c1 ; *c1ccc(C#N)c(C(F)(F)F)c1 ; *c1cc(F)c(C#N)cc1[N+](=O)[O-] ; *c1ccc(C#N)cc1C(C)=O ; *c1cc(F)c(C#N)cc1F ; *c1ccc(C#N)c2ccoc12]

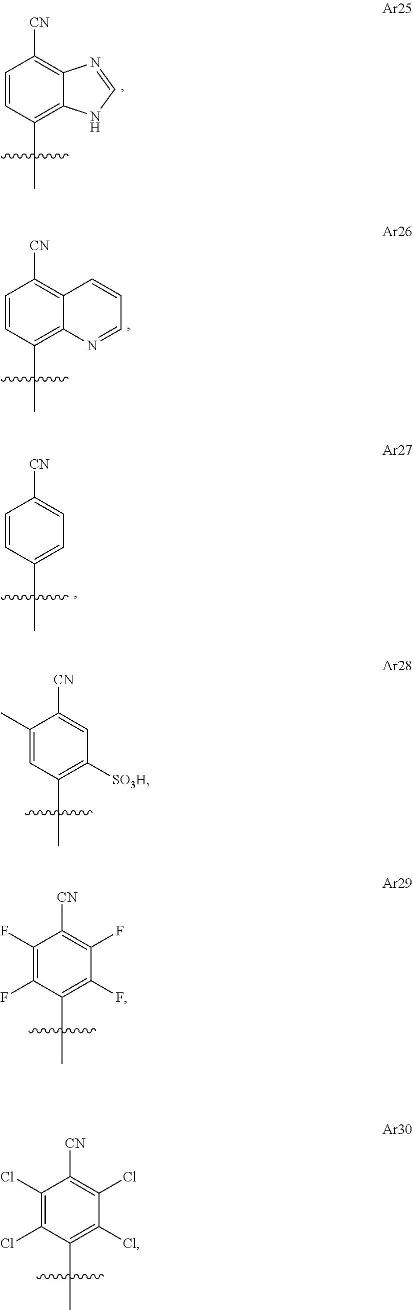 [SMILES: *c1ccc(C#N)c2nc[nH]c12 ; *c1ccc(C#N)c2cccnc12 ; *c1ccc(C#N)cc1 ; *c1cc(C)c(C#N)cc1S(=O)(=O)O ; *c1c(F)c(F)c(C#N)c(F)c1F ; *c1c(Cl)c(Cl)c(C#N)c(Cl)c1Cl]

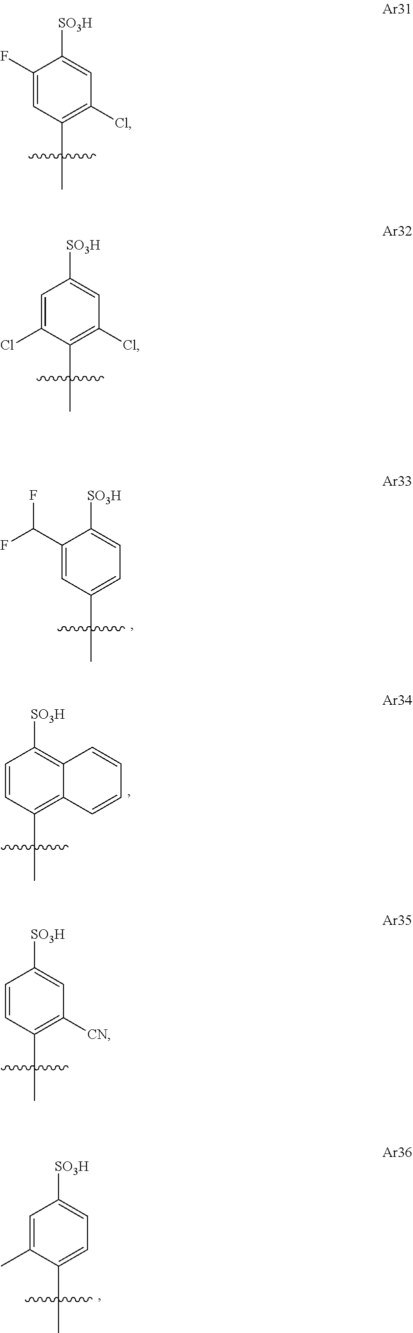 [SMILES: *c1cc(F)c(S(=O)(=O)O)cc1Cl ; *c1c(Cl)cc(S(=O)(=O)O)cc1Cl ; *c1ccc(S(=O)(=O)O)c(C(F)F)c1 ; *c1ccc(S(=O)(=O)O)c2ccccc12 ; *c1ccc(S(=O)(=O)O)cc1C#N ; *c1ccc(S(=O)(=O)O)cc1C]

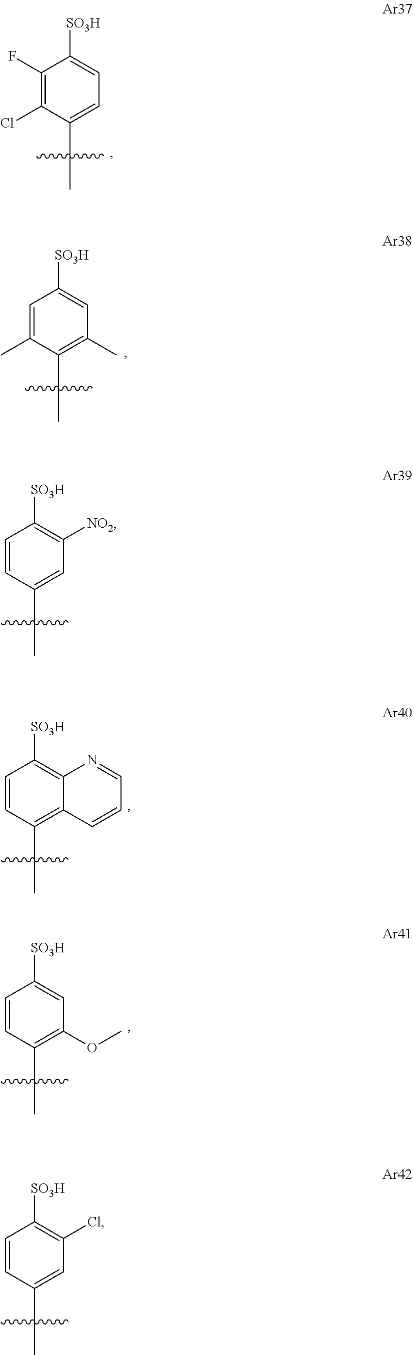

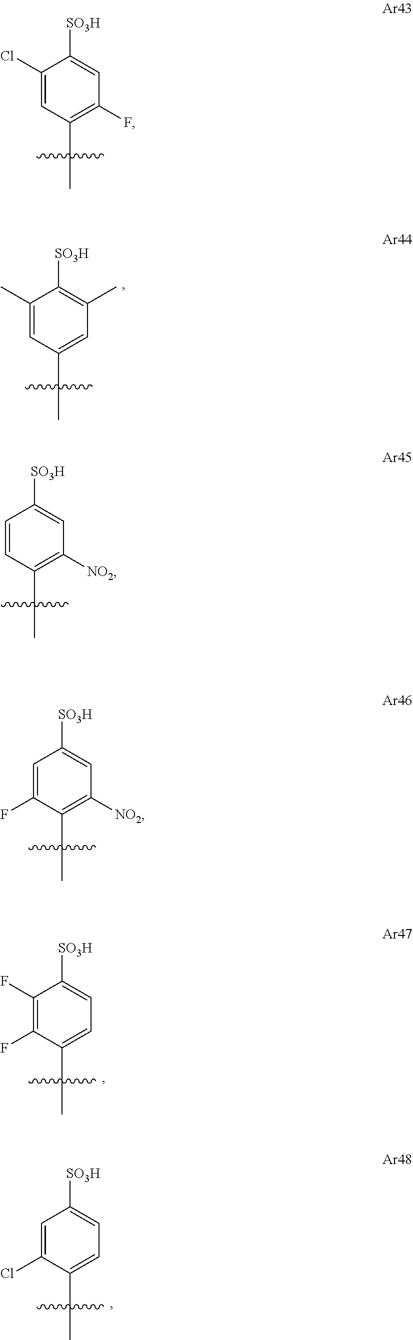

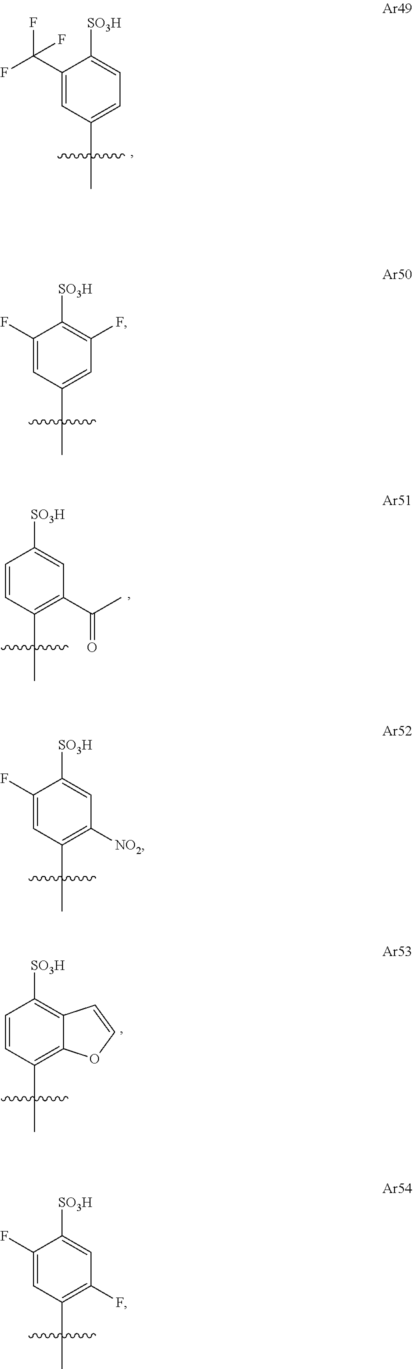 [SMILES: *c1ccc(S(=O)(=O)O)c(C(F)(F)F)c1 ; *c1cc(F)c(S(=O)(=O)O)c(F)c1 ; *c1ccc(S(=O)(=O)O)cc1C(C)=O ; *c1cc(F)c(S(=O)(=O)O)cc1[N+](=O)[O-] ; *c1ccc(S(=O)(=O)O)c2ccoc12 ; *c1cc(F)c(S(=O)(=O)O)cc1F]

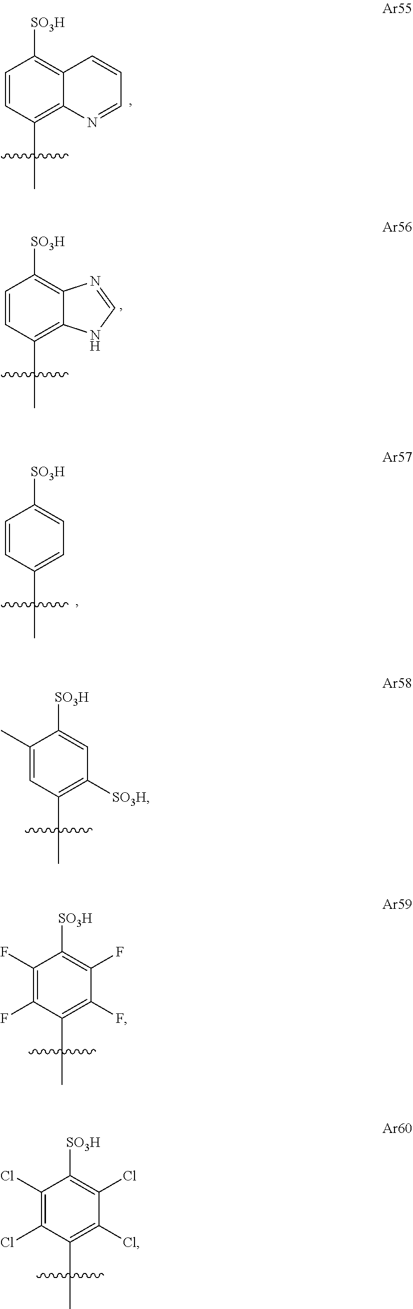 [SMILES: *c1ccc(S(=O)(=O)O)c2cccnc12 ; *c1ccc(S(=O)(=O)O)c2nc[nH]c12 ; *c1ccc(S(=O)(=O)O)cc1 ; *c1cc(C)c(S(=O)(=O)O)cc1S(=O)(=O)O ; *c1c(F)c(F)c(S(=O)(=O)O)c(F)c1F ; *c1c(Cl)c(Cl)c(S(=O)(=O)O)c(Cl)c1Cl]

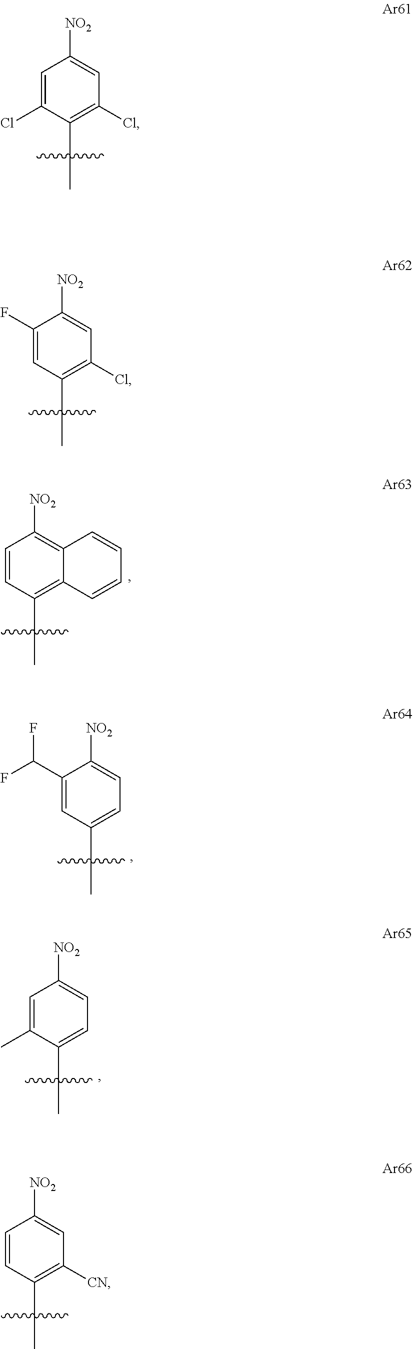 [SMILES: *c1c(Cl)cc([N+](=O)[O-])cc1Cl ; *c1cc(F)c([N+](=O)[O-])cc1Cl ; *c1ccc([N+](=O)[O-])c2ccccc12 ; *c1ccc([N+](=O)[O-])c(C(F)F)c1 ; *c1ccc([N+](=O)[O-])cc1C ; *c1ccc([N+](=O)[O-])cc1C#N]

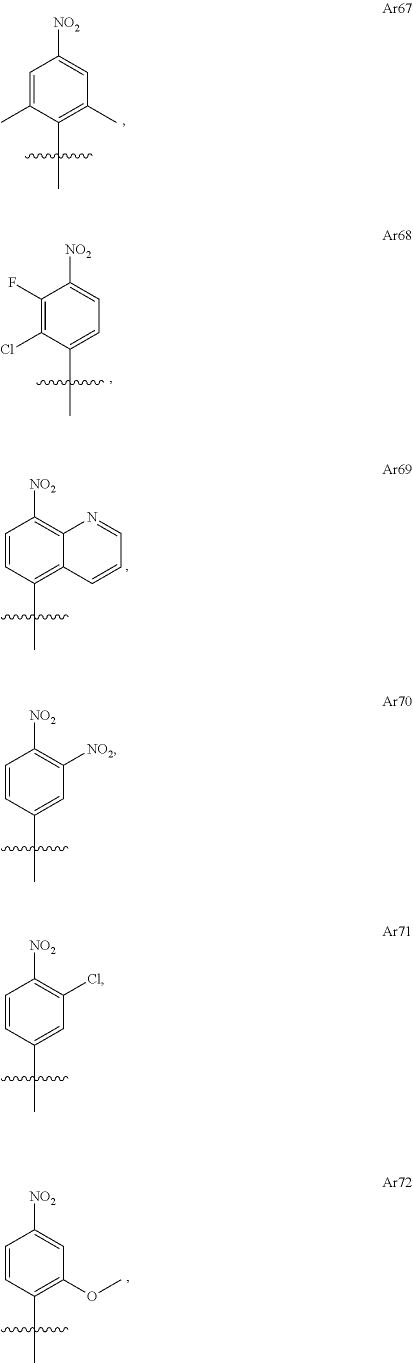

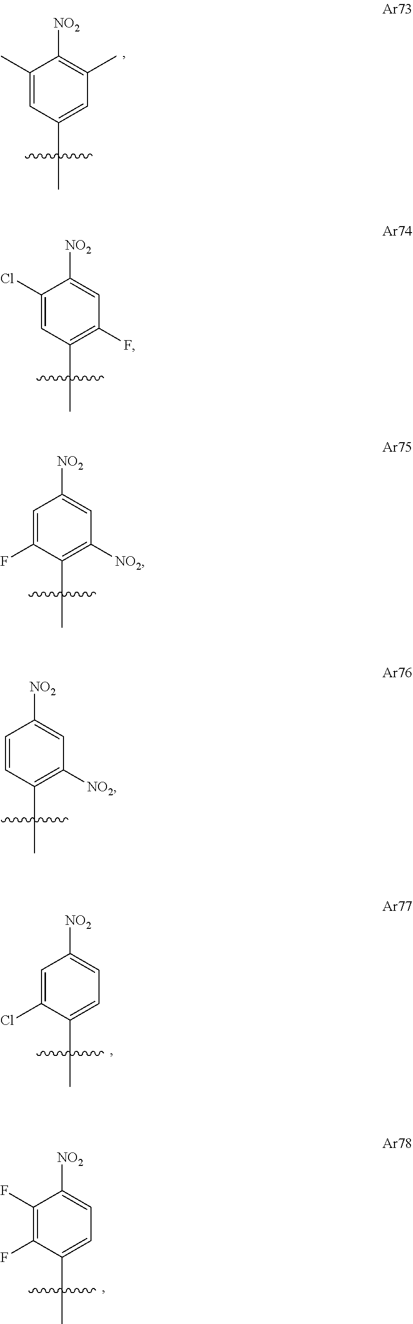

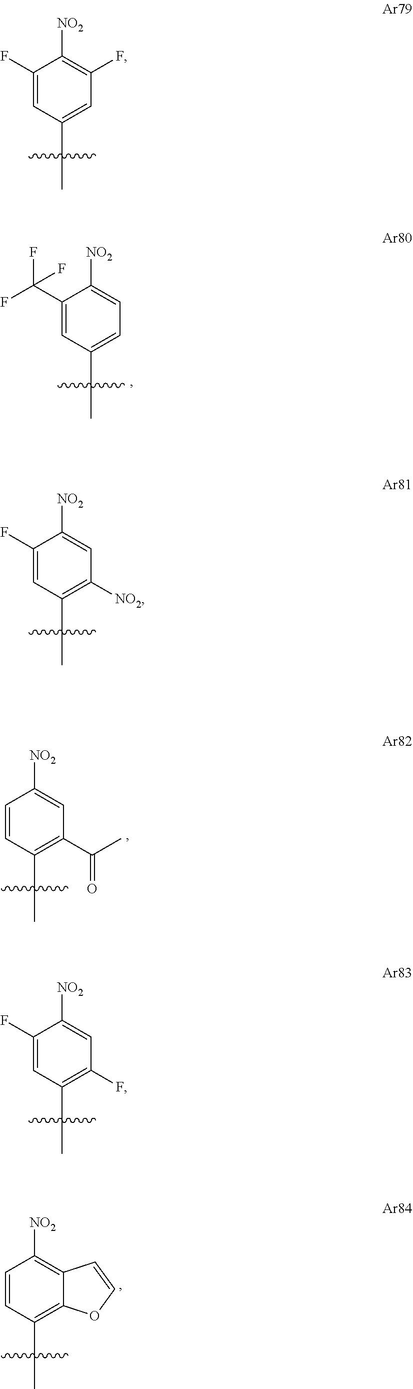 [SMILES: *c1cc(F)c([N+](=O)[O-])c(F)c1 ; *c1ccc([N+](=O)[O-])c(C(F)(F)F)c1 ; *c1cc(F)c([N+](=O)[O-])cc1[N+](=O)[O-] ; *c1ccc([N+](=O)[O-])cc1C(C)=O ; *c1cc(F)c([N+](=O)[O-])cc1F ; *c1ccc([N+](=O)[O-])c2ccoc12]

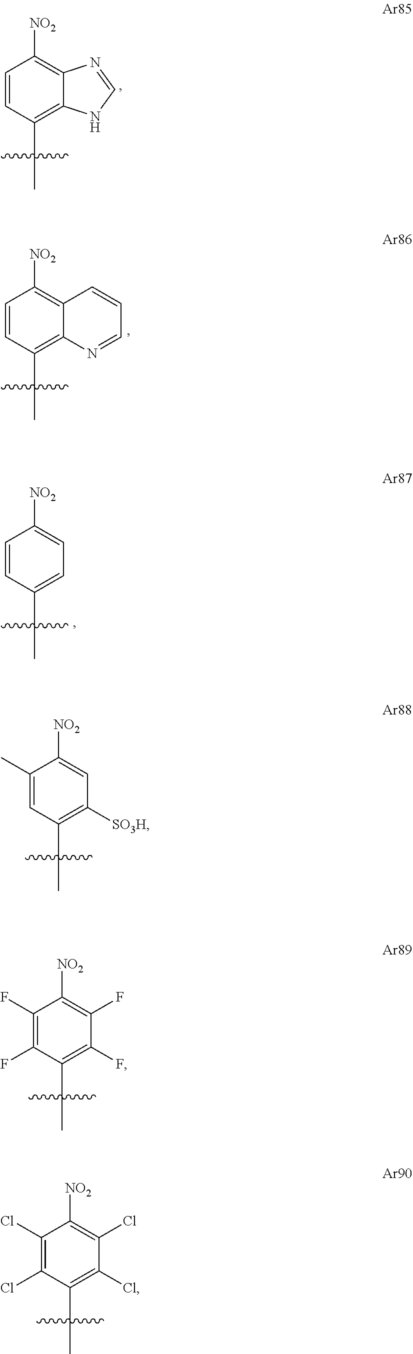 [SMILES: *c1ccc([N+](=O)[O-])c2nc[nH]c12 ; *c1ccc([N+](=O)[O-])c2cccnc12 ; *c1ccc([N+](=O)[O-])cc1 ; *c1cc(C)c([N+](=O)[O-])cc1S(=O)(=O)O ; *c1c(F)c(F)c([N+](=O)[O-])c(F)c1F ; *c1c(Cl)c(Cl)c([N+](=O)[O-])c(Cl)c1Cl]

or salts thereof.

8. The method of any one of aspects 1-7, wherein r is an integer from 1 to 10.

9. The method of any one of aspects 1-8, wherein r is an integer from 1 to 4.

10. The method of any one of aspects 1-9, wherein the linker comprises at least one ethylene glycol unit.

11. The method of any one of aspects 1-10, wherein the linker comprises at least five ethylene glycol units.

12. The method of any one of aspects 1-11, wherein the therapeutic agent is an immune agonist.

13. The method of any one of aspects 1-11, wherein the therapeutic agent is a TLR agonist.

14. The method of aspect 13, wherein the TLR agonist is selected from the group consisting of a TLR7 agonist, a TLR8 agonist, and a TLR7/TLR8 agonist.

15. The method of any one of aspects 1-11, wherein the therapeutic agent is an immune antagonist.

16. The method of any one of aspects 1-15, wherein the antibody construct is an antibody.

17. The method of aspect 16, wherein the antibody is an IgG1 antibody.

18. The method of any one of aspects 1-17, wherein the antibody construct comprises an antigen binding domain that binds to an antigen selected from the group consisting of CCR8, CDH1, CD19, CD20, CD24, CD29, CD30, CD38, CD40, CD47, EpCAM, MUC1, MUC16, MSLN, PD-L1, EGFR, VEGF, HER2, SLAMF7, PDGFRa, gp75, TROP2, PSMA, 5T4, ANGPT2, ANPEP, B7H3, B7H4, BCMA, CA9, CD125, CD37, CD74, CLDN3, CLEC11A, CLEC5A, CLEC6A, CTAG1B, CTAL4, EPHA2, EPHA4, FGFR3, FOLR1, GD2, GPC3, GPNMB, HLA-DRA, IL-13, IL3RA2, KITLG, L1CAM, LAG3, Lewis-Y antigen, LILRB1, LRRC15, MAGEA3, MAGEA6, MUC1, MUC16, NOTCH, NRP1, NY-ESO-1, P2RX7, PCD1, PSCA, PVRIG, ROR1, SIGLEC10, SIGLEC11, SIGLEC12, SIGLEC14, SIGLEC15, SIGLEC5, SIGLEC6, SIGLEC7, SIGLEC8, SIGLEC9, SIRPA, SLAMF7, SLC39A6, TNFSF10, and WT1.

19. An immunoconjugate or salt thereof prepared from the method of any one of aspects 1-18.

20. A composition comprising a plurality of immunoconjugates or salts thereof prepared from the method of any one of aspects 1-18.

21. A method of treating or preventing a disease or condition comprising administering a therapeutically effective amount of an immunoconjugate or salt thereof according to aspect 19 or a composition according to aspect 20 to a subject in need thereof.

22. A method of treating or preventing cancer comprising administering a therapeutically effective amount of an immunoconjugate or salt thereof according to aspect 19 or a composition according to aspect 20 to a subject in need thereof.

23. The method of aspect 21, wherein the disease or condition is susceptible to an immune response resulting from a therapeutic agent that is an immune antagonist.

24. The method of aspect 21, wherein the disease or condition is susceptible to an immune response resulting from a therapeutic agent that is an immune agonist.

25. The method of aspect 22, wherein the cancer is susceptible to an immune response resulting from a therapeutic agent that is an immune antagonist.

26. The method of aspect 22, wherein the cancer is susceptible an immune response resulting from a therapeutic agent that is an immune agonist.

27. The method of aspect 22, wherein the cancer is susceptible to a pro-inflammatory response induced by TLR7 and/or TLR8 agonism.

### EXAMPLES

The following examples further illustrate the invention but, of course, should not be construed as in any way limiting its scope.

### Example 1: c Log P Calculation

This example shows that a therapeutic agent linker compound having a sulfo-tetrafluorophenyl ester (S-TFP; Ar59) or a sulfo-dichlorophenyl ester (SDP; Ar32) should have a higher water solubility than a therapeutic agent linker compound having a tetrafluorophenyl ester (TFP ester), as evidenced by c Log P calculations of S-TFP, SDP, and TFP.

The Log P value refers to the logarithmic assessment of a compounds partition coefficient between n-octanol and water, i.e., log(c_{octanol}/c_{water}). Thus a negative Log P value indicates that a compound is more likely to partition into water, i.e., is more soluble in water than in octanol. c Log P is a theoretical calculation utilized to assess the hydrophilicity of a compound based on its chemical composition.

The c Log P values for TFP, S-TFP, and SDP were calculated using ChemDraw™, and the resulting c Log P values are shown below.

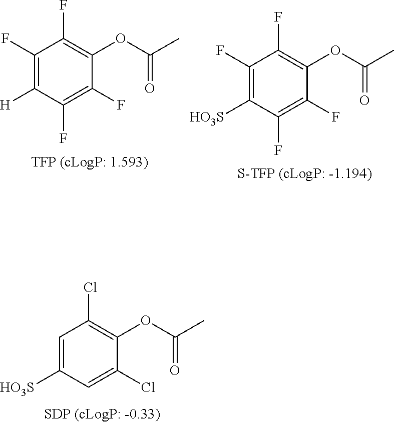

As shown above, TFP has a positive c Log P value, indicating that TFP is hydrophobic and is more likely to partition into octanol than water. In contrast, S-TFP and SDP have a negative c Log P value, indicating that a therapeutic agent linker compound having a sulfo-tetrafluorophenyl ester (S-TFP) or a sulfo-dichlorophenyl ester (SDP) should have a higher water solubility than a therapeutic agent linker compound having a tetrafluorophenyl ester (TFP ester). In addition, the c Log P value for sulfo-tetrafluorophenyl ester (S-TFP) is more negative than sulfo-dichlorophenyl ester (SDP), which shows that a therapeutic agent linker compound having a sulfo-tetrafluorophenyl ester (S-TFP) should have a higher water solubility than a therapeutic agent linker compound having a sulfo-tetrafluorophenyl ester (S-TFP).

### Example 2: Stability Analysis

This example shows that a therapeutic agent linker compound having a sulfo-tetrafluorophenyl ester (S-TFP; Ar59) is more reactive than a therapeutic agent linker compound having a tetrafluorophenyl ester (TFP ester), as evidenced by its hydrolytic instability in a conjugation buffer.

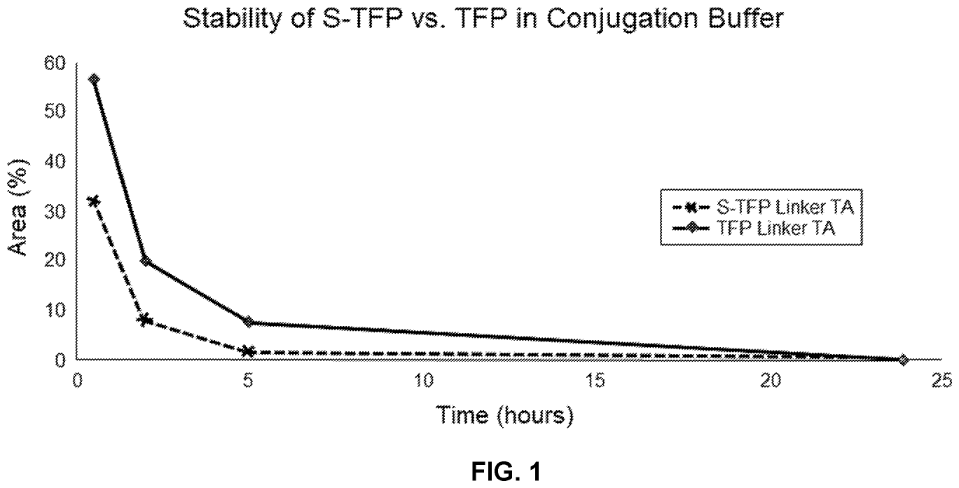

25 mM solutions of a therapeutic agent linker compound having a sulfo-tetrafluorophenyl ester (S-TFP Linker TA) or a therapeutic agent linker compound having a tetrflurophenyl ester (TFP Linker TA) were prepared by adding 106 μL of dimethylacetamide (DMA) to the corresponding therapeutic agent linker compound in a 1 mL vial. The resulting solutions were stirred at room temperature. 15 μL of the S-TFP Linker TA solution and the TFP Linker TA were added to two separate vials containing 0.9 mL of a borate buffer (pH 8.3). The resulting conjugation buffer solutions were separately monitored using high performance liquid chromatography (HPLC) at 0 hours, 2 hours, 5 hours, and 25 hours. The relative amounts (area %) of S-TFP Linker TA and TFP Linker TA were plotted as a function of time as shown in FIG. 1.

As demonstrated by the data presented in FIG. 1, TFP Linker TA is hydrolytically more stable than S-TFP Linker TA at all times measured, as evidenced by the higher area % of the therapeutic agent linker compound. Since TFP Linker TA is hydrolytically more stable than S-TFP Linker TA, S-TFP Linker TA should be more reactive than TFP Linker TA.

### Example 3: Conjugation Profile Analysis

This example shows that sulfo-tetrafluorophenyl ester (S-TFP; Ar59) and sulfo-dichlorophenyl ester (SDP; Ar32) provide a different conjugation profile than a tetrafluorophenyl ester (TFP ester).

Trastuzumab was buffer exchanged into the conjugation buffer containing 100 mM boric acid, 50 mM sodium chloride, 1 mM ethylenediaminetetraacetic acid at pH 8.3, using G-25 SEPHADEX™ desalting columns (Sigma-Aldrich). The eluates were then each adjusted to 6 mg/ml using the buffer and sterile filtered. Trastuzumab at 6 mg/ml was pre-warmed to 30° C. and rapidly mixed with 8 molar equivalents of therapeutic agent/linker moieties terminated in each of esters TFP, NHS, S-TFP, and SDP.

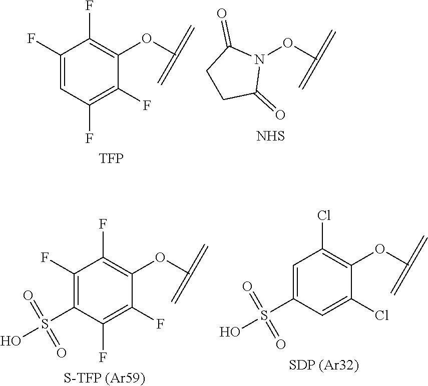

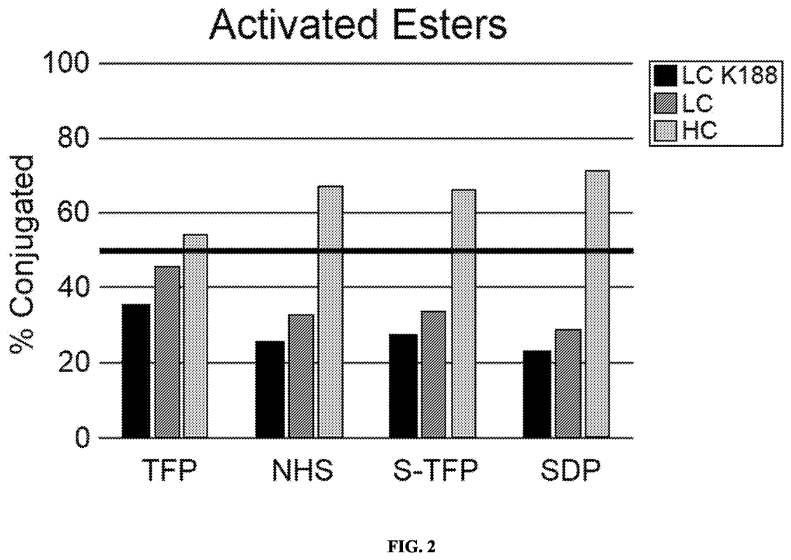

The reaction was allowed to proceed for 16 hours at 30° C. and the resulting immunoconjugates were separated from reactants by running over two successive G-25 desalting columns equilibrated in PBS at pH 7.2. The resulting immunoconjugates were buffer exchanged to 2 mg/ml in 50 mM Tris creating a final volume of 20 μL. To the buffer exchanged solutions was added 10 μL of 50 mM stock dithiothreitol and the resulting mixture was incubated for 60 minutes at 37° C. on a shaker. Peptide mapping of the light chain (LC) and the heavy chain (HC) was carried out by injecting the digested samples onto a C4 reverse phase column on an ACQUITY™ UPLC H-class system (Waters Corporation) connected to a XEVO™ G2-XS TOF mass spectrometer (Waters Corporation). Specific conjugating residues, and their relative abundance, were determined by further digesting the reduced immunoconjugate with trypsin prior to injecting the samples onto the C4 reverse phase column. The conjugation results are provided in FIG. 2.

As demonstrated by the data presented in FIG. 2, conjugation with S-TFP and SDP resulted in an increase in selectivity (i.e., percent conjugation) for the heavy chain, relative to conjugation with TFP. The amount of heavy chain conjugation (i.e., approximately 65% to 75%) is similar to conjugation with an NHS ester. However, due to relative instability, the NHS conjugation produced a reduced yield of the desired immunoconjugate. In addition, S-TFP and SDP resulted in significantly reduced conjugation at LC K188.

These results support the conclusions of Examples 1 and 2, i.e., that therapeutic agent linker compounds having a sulfo-tetrafluorophenyl ester (S-TFP; Ar59) or a sulfo-dichlorophenyl ester (SDP; Ar32) have significantly different reactivity patterns as a result of their solubility and/or their reactivity.

### Example 4: Solubility Analysis

This example shows the effect of solubility of therapeutic agent/linker moiety on average therapeutic agent to antibody ratio of a composition of immunoconjugates formed from conjugation with a sulfo-tetrafluorophenyl ester (S-TFP; Ar59) ester.

Trastuzumab was buffer exchanged into the conjugation buffer containing 100 mM boric acid, 50 mM sodium chloride, and 1 mM ethylenediaminetetraacetic acid at pH 8.3, using G-25 SEPHADEX™ desalting columns (Sigma-Aldrich). The eluates were then each adjusted to 6 mg/ml Trastuzumab using the buffer. The resulting mixtures were sterile filtered, pre-warmed to 30° C., and rapidly mixed with a buffer solution containing 8 molar equivalents of therapeutic agent/linker moieties terminated in S-TFP and certain percentages (i.e., 9 v/v % or 16 v/v %) of dimethylacetamide (DMA) or dimethylsulfoxide (DMSO). The turbidity of the therapeutic agent/linker moiety solution and the therapeutic agent to antibody ratio of the resulting immunoconjugate were measured, as outlined in Table 1.

TABLE 1
Solubility Analysis

| | Turbidity | Therapeutic Agent to Anti- |
|---|---|---|
| Additive (v/v %) | (Abs 600 nm) | body Ratio (Average) |
| None | 10.7 | 0.58 |
| DMA (9) | 5.6 | 2.26 |
| DMA (16) | 2.2 | 4.48 |
| DMSO (9) | 7.4 | 0.90 |
| DMSO (16) | 4.3 | 2.66 |

As demonstrated by the results set forth in Table 1, solubility of the therapeutic agent/linker moiety plays a crucial role in the therapeutic agent to antibody ratio of the resulting immunoconjugate. As is apparent from the results in Table 1, as the solubility increases (i.e., turbidity decreases), the average therapeutic agent to antibody ratio increases.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for producing an immunoconjugate, the method comprising combining one or more compounds of Formula I: 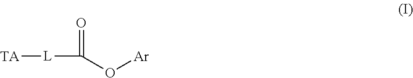
or salts thereof,
and an antibody construct of Formula II: 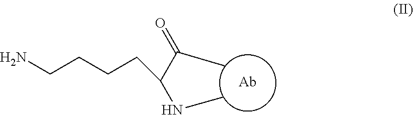
or salt thereof,
wherein Formula II is an antibody construct with residue 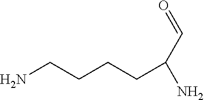
representing one or more lysine residues of the antibody construct,
to provide the immunoconjugate of Formula III: 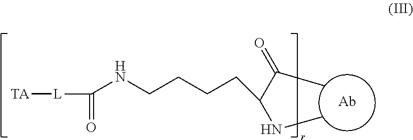
or salt thereof,
wherein
TA is a therapeutic agent,
L is a linker,
r is an integer from 1 to 50,
Ar is an aromatic moiety comprising a first substituent selected from PEG, —SO₂CX₃, —NR₃⁺, —NO₂, —SO₃R, —SO₂R, —CN, —CX₃, —PO₃R₂, —OPO₃R₂, 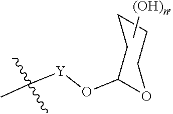
and salts thereof
each R independently is H, CX₃, or C₁-C₄ alkyl,
each X independently is hydrogen or a halogen,
Y is CH₂, PEG, or a bond,
n is an integer from 1 to 4, and
PEG has the formula:
—(CH₂CH₂O)ₘ—(CH₂)ₚ—,
where p is an integer from 1 to 5 and m is an integer from 2 to 50.

2. The method of claim 1, wherein Ar further comprises one or more additional substituents selected from —F, —Cl, —Br, —I, —CR₃, —OR, —C(O)R, —C(O)OR, PEG, —SO₂CX₃, —NR₃⁺, —NO₂, —SO₃R, —SO₂R, —CN, —CX₃, —PO₃R₂, —OPO₃R₂, 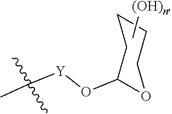
salts thereof, and combinations thereof,
wherein each R independently is H, CX₃, or C₁-C₄ alkyl,
each X independently is hydrogen or a halogen,
Y is CH₂, PEG, or a bond,
n is an integer from 1 to 4, and
PEG has the formula:
—(CH₂CH₂O)ₘ—(CH₂)ₚ—,
where p is an integer from 1 to 5 and m is an integer from 2 to 50.

3. The method of claim 1, wherein the first substituent is selected from —NO₂, —SO₃H, —CN, and salts thereof.

4. The method of claim 1, wherein the first substituent is —SO₃H or a salt thereof.

5. The method of claim 2, wherein the one or more additional substituents is selected from —F, —Cl, —Br, —I, —NO₂, —SO₃H, —CN, and salts thereof.

6. The method of claim 2, wherein the one or more additional substituents is selected from —F, —Cl, —Br, and —I.

7. The method of claim 1, wherein Ar is of formula: 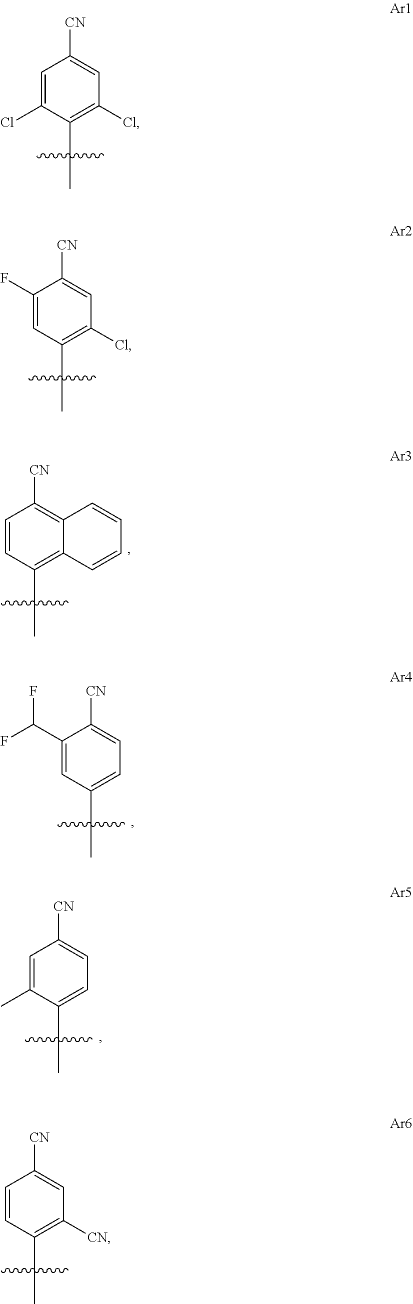 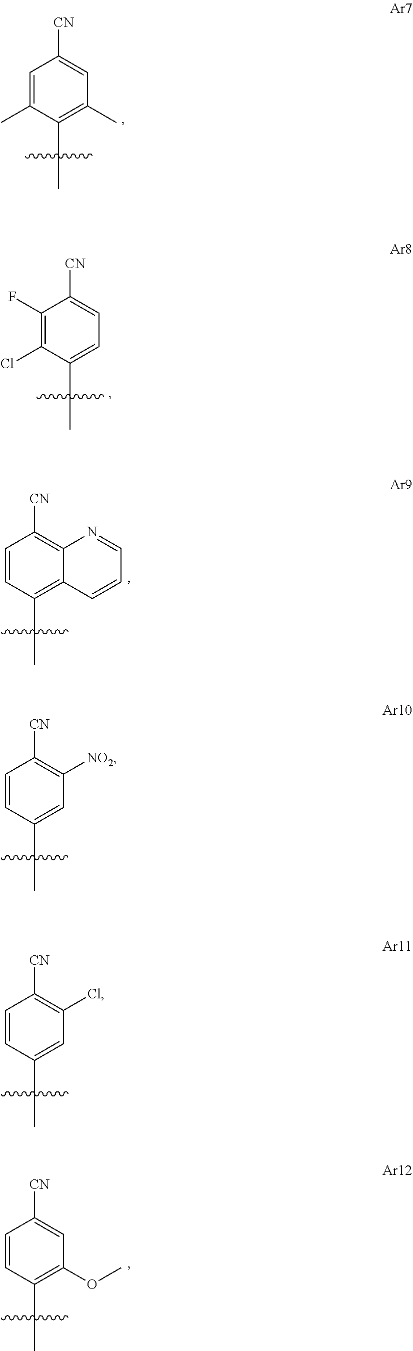 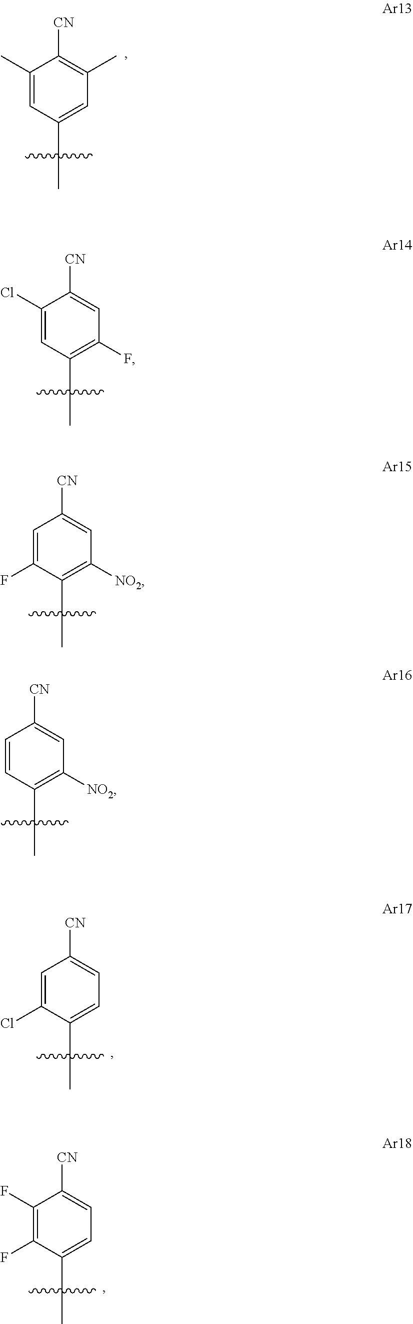 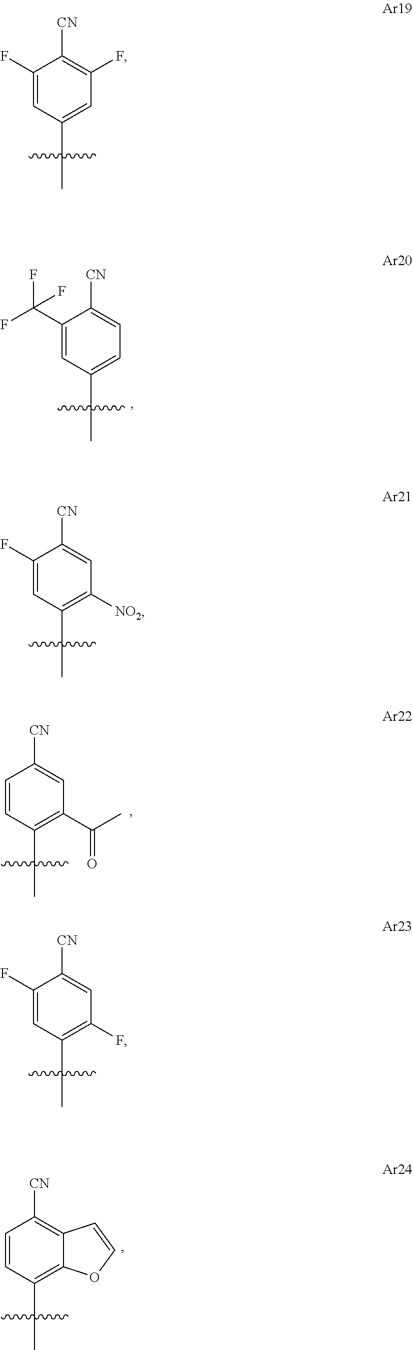 [SMILES: *c1cc(F)c(C#N)c(F)c1 ; *c1ccc(C#N)c(C(F)(F)F)c1 ; *c1cc(F)c(C#N)cc1[N+](=O)[O-] ; *c1ccc(C#N)cc1C(C)=O ; *c1cc(F)c(C#N)cc1F ; *c1ccc(C#N)c2ccoc12] 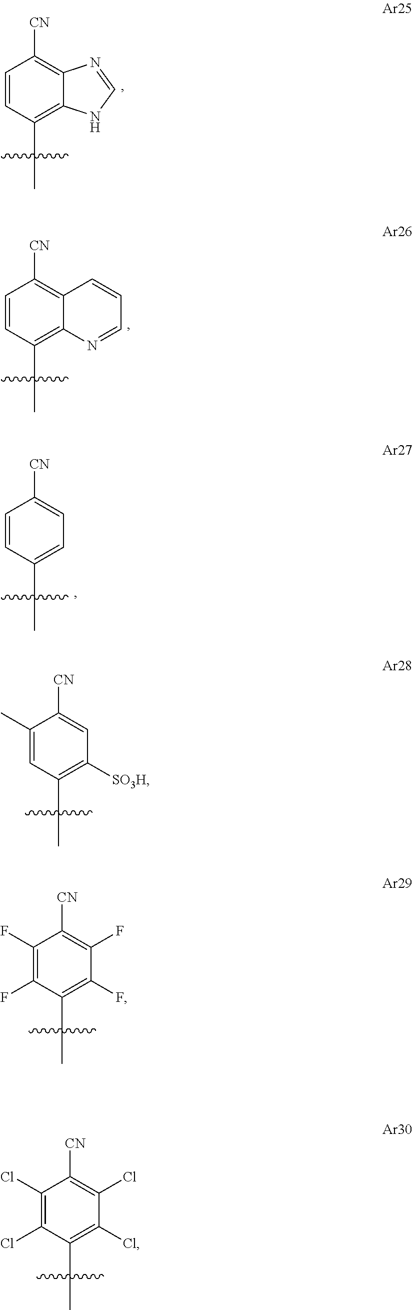 [SMILES: *c1ccc(C#N)c2nc[nH]c12 ; *c1ccc(C#N)c2cccnc12 ; *c1ccc(C#N)cc1 ; *c1cc(C)c(C#N)cc1S(=O)(=O)O ; *c1c(F)c(F)c(C#N)c(F)c1F ; *c1c(Cl)c(Cl)c(C#N)c(Cl)c1Cl] 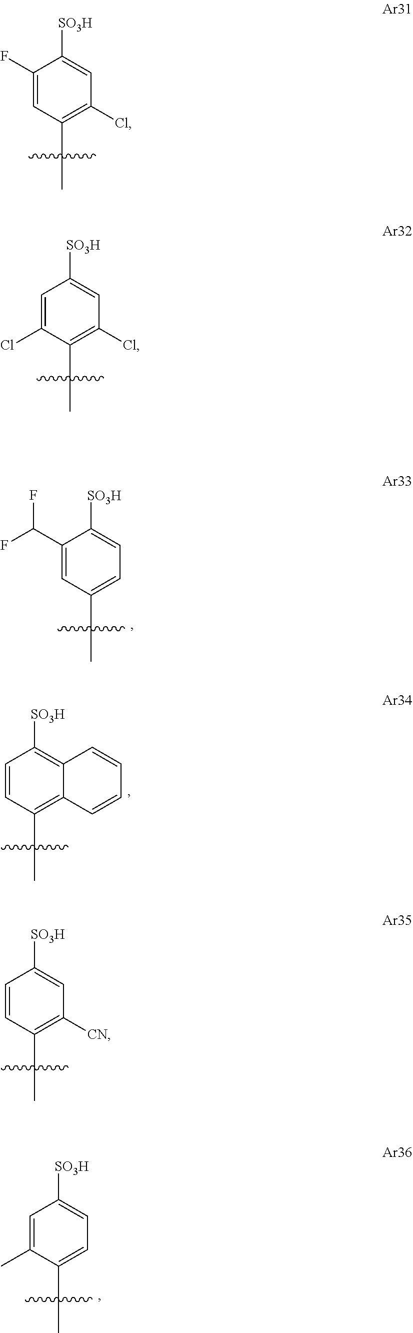 [SMILES: *c1cc(F)c(S(=O)(=O)O)cc1Cl ; *c1c(Cl)cc(S(=O)(=O)O)cc1Cl ; *c1ccc(S(=O)(=O)O)c(C(F)F)c1 ; *c1ccc(S(=O)(=O)O)c2ccccc12 ; *c1ccc(S(=O)(=O)O)cc1C#N ; *c1ccc(S(=O)(=O)O)cc1C] 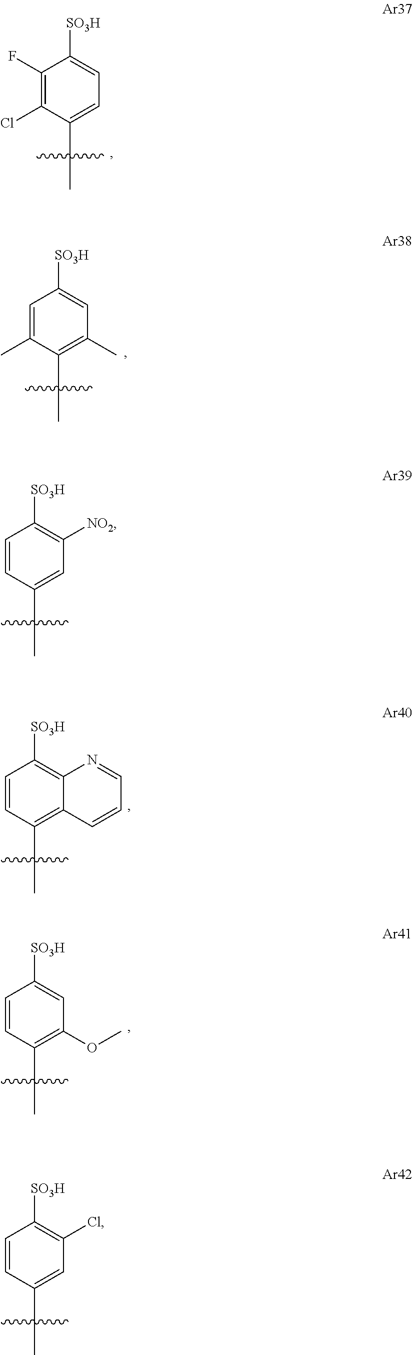 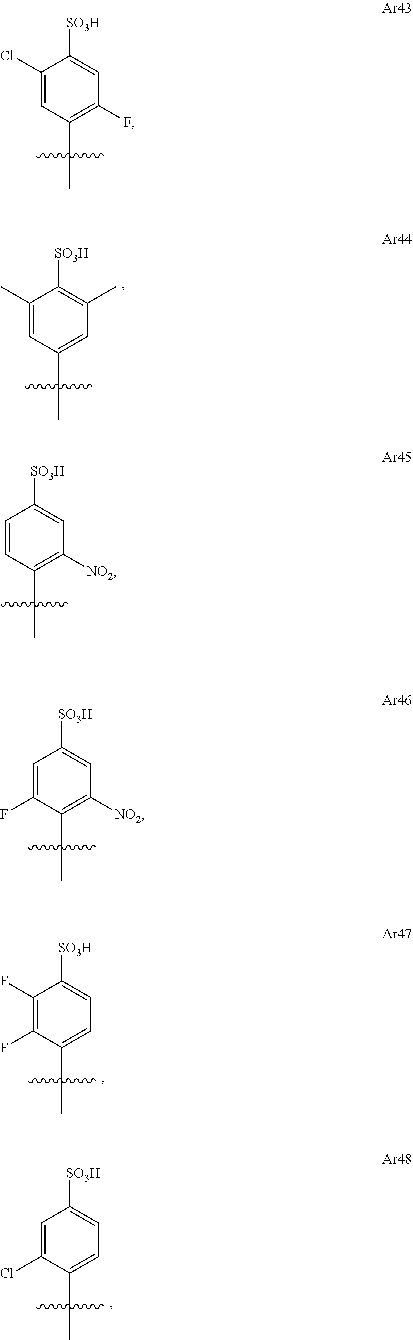 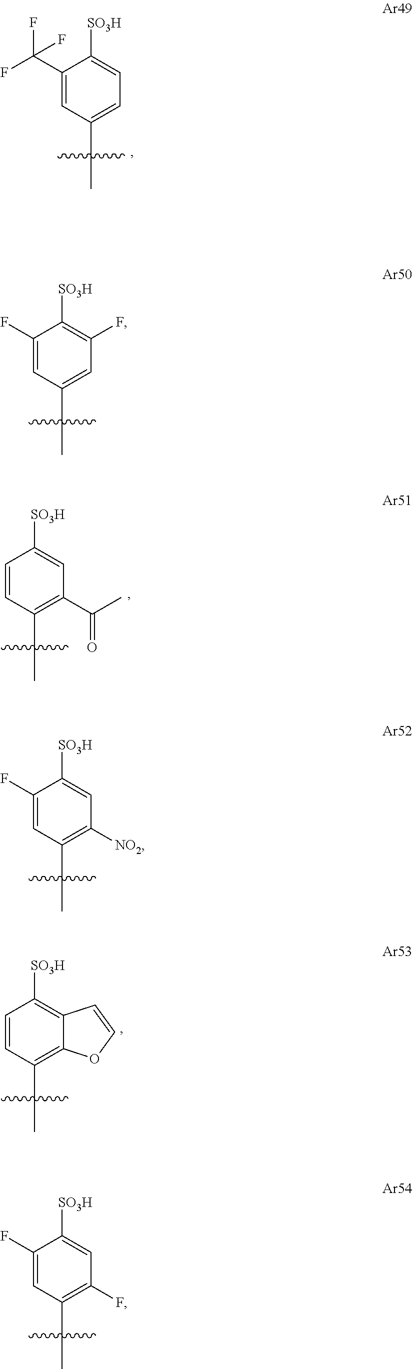 [SMILES: *c1ccc(S(=O)(=O)O)c(C(F)(F)F)c1 ; *c1cc(F)c(S(=O)(=O)O)c(F)c1 ; *c1ccc(S(=O)(=O)O)cc1C(C)=O ; *c1cc(F)c(S(=O)(=O)O)cc1[N+](=O)[O-] ; *c1ccc(S(=O)(=O)O)c2ccoc12 ; *c1cc(F)c(S(=O)(=O)O)cc1F] 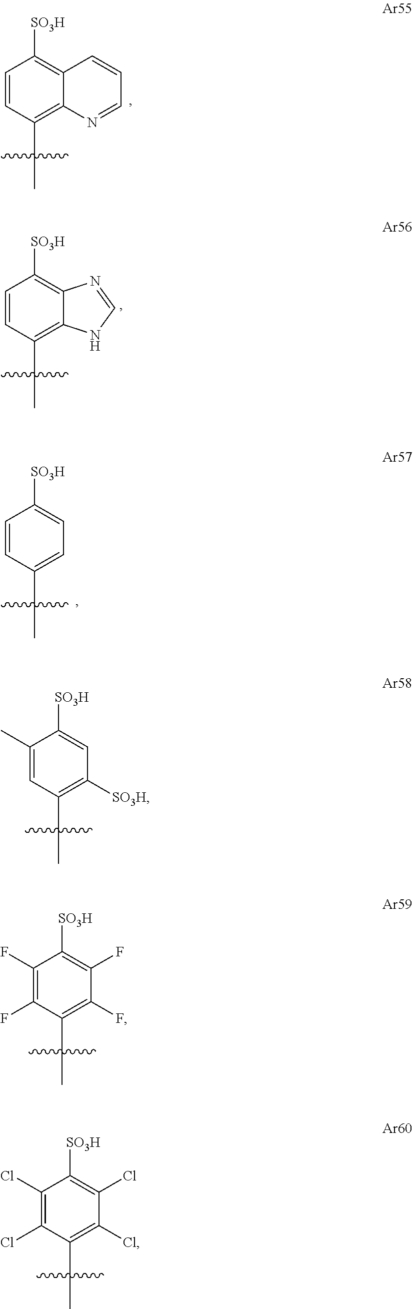 [SMILES: *c1ccc(S(=O)(=O)O)c2cccnc12 ; *c1ccc(S(=O)(=O)O)c2nc[nH]c12 ; *c1ccc(S(=O)(=O)O)cc1 ; *c1cc(C)c(S(=O)(=O)O)cc1S(=O)(=O)O ; *c1c(F)c(F)c(S(=O)(=O)O)c(F)c1F ; *c1c(Cl)c(Cl)c(S(=O)(=O)O)c(Cl)c1Cl] 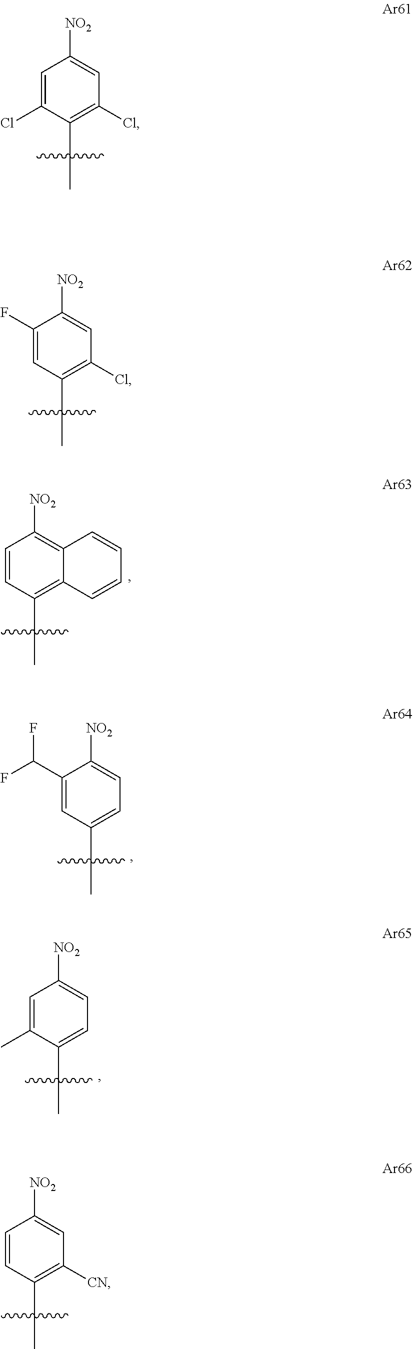 [SMILES: *c1c(Cl)cc([N+](=O)[O-])cc1Cl ; *c1cc(F)c([N+](=O)[O-])cc1Cl ; *c1ccc([N+](=O)[O-])c2ccccc12 ; *c1ccc([N+](=O)[O-])c(C(F)F)c1 ; *c1ccc([N+](=O)[O-])cc1C ; *c1ccc([N+](=O)[O-])cc1C#N] 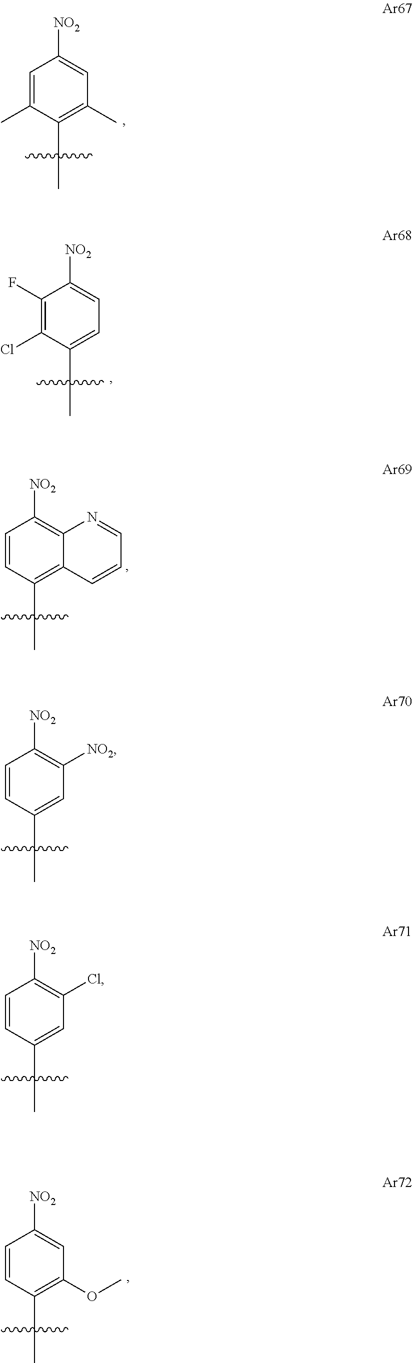 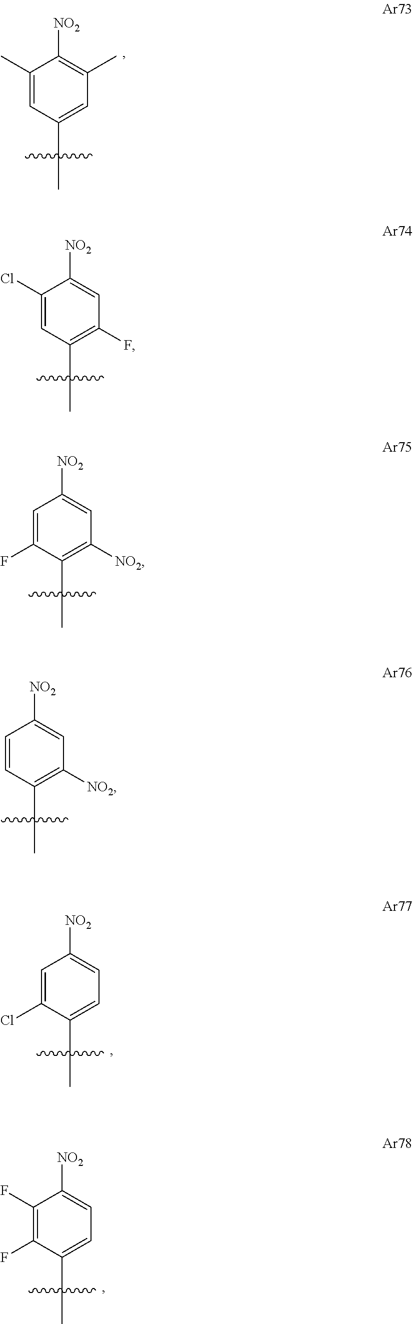 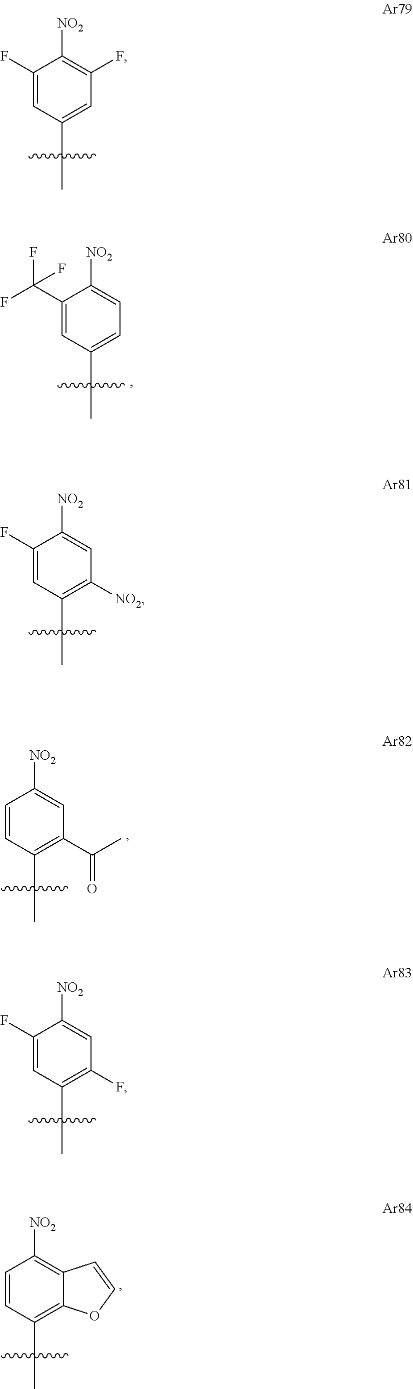 [SMILES: *c1cc(F)c([N+](=O)[O-])c(F)c1 ; *c1ccc([N+](=O)[O-])c(C(F)(F)F)c1 ; *c1cc(F)c([N+](=O)[O-])cc1[N+](=O)[O-] ; *c1ccc([N+](=O)[O-])cc1C(C)=O ; *c1cc(F)c([N+](=O)[O-])cc1F ; *c1ccc([N+](=O)[O-])c2ccoc12] 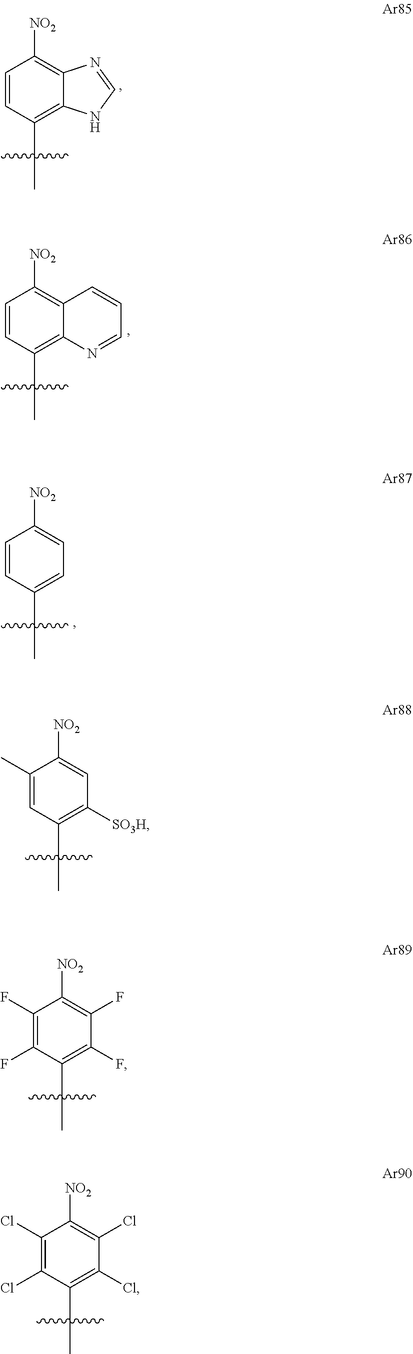 [SMILES: *c1ccc([N+](=O)[O-])c2nc[nH]c12 ; *c1ccc([N+](=O)[O-])c2cccnc12 ; *c1ccc([N+](=O)[O-])cc1 ; *c1cc(C)c([N+](=O)[O-])cc1S(=O)(=O)O ; *c1c(F)c(F)c([N+](=O)[O-])c(F)c1F ; *c1c(Cl)c(Cl)c([N+](=O)[O-])c(Cl)c1Cl]
or salts thereof.

8. The method of claim 1, wherein r is an integer from 1 to 10.

9. The method of claim 1, wherein r is an integer from 1 to 4.

10. The method of claim 1, wherein the linker comprises at least one ethylene glycol unit.

11. The method of claim 1, wherein the linker comprises at least five ethylene glycol units.

12. The method of claim 1, wherein the therapeutic agent is an immune agonist.

13. The method of claim 1, wherein the therapeutic agent is a TLR agonist.

14. The method of claim 13, wherein the TLR agonist is selected from the group consisting of a TLR7 agonist, a TLR8 agonist, and a TLR7/TLR8 agonist.

15. The method of claim 1, wherein the therapeutic agent is an immune antagonist.

16. The method of claim 1, wherein the antibody construct is an antibody.

17. (canceled)

18. The method of claim 1, wherein the antibody construct comprises an antigen binding domain that binds to an antigen selected from the group consisting of CCR8, CDH1, CD19, CD20, CD24, CD29, CD30, CD38, CD40, CD47, EpCAM, MUC1, MUC16, MSLN, PD-L1, EGFR, VEGF, HER2, SLAMF7, PDGFRa, gp75, TROP2, PSMA, 5T4, ANGPT2, ANPEP, B7H3, B7H4, BCMA, CA9, CD125, CD37, CD74, CLDN3, CLEC11A, CLEC5A, CLEC6A, CTAG1B, CTAL4, EPHA2, EPHA4, FGFR3, FOLR1, GD2, GPC3, GPNMB, HLA-DRA, IL-13, IL3RA2, KITLG, L1CAM, LAG3, Lewis-Y antigen, LILRB1, LRRC15, MAGEA3, MAGEA6, MUC1, MUC16, NOTCH, NRP1, NY-ESO-1, P2RX7, PCD1, PSCA, PVRIG, ROR1, SIGLEC10, SIGLEC11, SIGLEC12, SIGLEC14, SIGLEC15, SIGLEC5, SIGLEC6, SIGLEC7, SIGLEC8, SIGLEC9, SIRPA, SLAMF7, SLC39A6, TNFSF10, and WT1.

19. An immunoconjugate or salt thereof prepared from the method of claim 1.

20. A composition comprising a plurality of immunoconjugates or salts thereof prepared from the method of claim 1.

21. A method of treating or preventing a disease or condition comprising administering a therapeutically effective amount of an immunoconjugate or salt thereof according to claim 19 or a composition comprising a plurality of said immunoconjugates or salts thereof to a subject in need thereof.

22.-27. (canceled)


SEQUENCE LISTING (170 sequences)

SEQ ID NO: 1 - protein, 290 aa, Artificial Sequence
  Synthetic
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 11 aa, Artificial Sequence
  Synthetic
RASQDVSTAVA

SEQ ID NO: 3 - protein, 7 aa, Artificial Sequence
  Synthetic
SASFLYS

SEQ ID NO: 4 - protein, 9 aa, Artificial Sequence
  Synthetic
QQYLYHPAT

SEQ ID NO: 5 - protein, 5 aa, Artificial Sequence
  Synthetic
DSWIH

SEQ ID NO: 6 - protein, 17 aa, Artificial Sequence
  Synthetic
WISPYGGSTYYADSVKG

SEQ ID NO: 7 - protein, 9 aa, Artificial Sequence
  Synthetic
RHWPGGFDY

SEQ ID NO: 8 - protein, 23 aa, Artificial Sequence
  Synthetic
DIQMTQSPSSLSASVGDRVTITC

SEQ ID NO: 9 - protein, 15 aa, Artificial Sequence
  Synthetic
WYQQKPGKAPKLLIY

SEQ ID NO: 10 - protein, 32 aa, Artificial Sequence
  Synthetic
GVPSRFSGSGSGTDFTLTISSLQPEDFATYYC

SEQ ID NO: 11 - protein, 117 aa, Artificial Sequence
  Synthetic
FGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 30 aa, Artificial Sequence
  Synthetic
EVQLVESGGGLVQPGGSLRLSCAASGFTFS

SEQ ID NO: 13 - protein, 14 aa, Artificial Sequence
  Synthetic
WVRQAPGKGLEWVA

SEQ ID NO: 14 - protein, 32 aa, Artificial Sequence
  Synthetic
RFTISADTSKNTAYLQMNSLRAEDTAVYYCAR

SEQ ID NO: 15 - protein, 341 aa, Artificial Sequence
  Synthetic
WGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 12 aa, Artificial Sequence
  Synthetic
RASQRVSSSYLA

SEQ ID NO: 17 - protein, 7 aa, Artificial Sequence
  Synthetic
DASSRAT

SEQ ID NO: 18 - protein, 9 aa, Artificial Sequence
  Synthetic
QQYGSLPWT

SEQ ID NO: 19 - protein, 5 aa, Artificial Sequence
  Synthetic
RYWMS

SEQ ID NO: 20 - protein, 17 aa, Artificial Sequence
  Synthetic
NIKQDGSEKYYVDSVKG

SEQ ID NO: 21 - protein, 12 aa, Artificial Sequence
  Synthetic
EGGWFGELAFDY

SEQ ID NO: 22 - protein, 23 aa, Artificial Sequence
  Synthetic
EIVLTQSPGTLSLSPGERATLSC

SEQ ID NO: 23 - protein, 15 aa, Artificial Sequence
  Synthetic
WYQQKPGQAPRLLIY

SEQ ID NO: 24 - protein, 32 aa, Artificial Sequence
  Synthetic
GIPDRFSGSGSGTDFTLTISRLEPEDFAVYYC

SEQ ID NO: 25 - protein, 117 aa, Artificial Sequence; identical to SEQ ID NO: 11
  Synthetic

SEQ ID NO: 26 - protein, 30 aa, Artificial Sequence; identical to SEQ ID NO: 12
  Synthetic

SEQ ID NO: 27 - protein, 14 aa, Artificial Sequence
  Synthetic
WVRQAPGKGLEWVA

SEQ ID NO: 28 - protein, 32 aa, Artificial Sequence
  Synthetic
RFTISRDNAKNSLYLQMNSLRAEDTAVYYCAR

SEQ ID NO: 29 - protein, 341 aa, Artificial Sequence
  Synthetic
WGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 14 aa, Artificial Sequence
  Synthetic
TGTSSDVGGYNYVS

SEQ ID NO: 31 - protein, 7 aa, Artificial Sequence
  Synthetic
DVSNRPS

SEQ ID NO: 32 - protein, 10 aa, Artificial Sequence
  Synthetic
SSYTSSSTRV

SEQ ID NO: 33 - protein, 5 aa, Artificial Sequence
  Synthetic
SYIMM

SEQ ID NO: 34 - protein, 17 aa, Artificial Sequence
  Synthetic
SIYPSGGITFYADTVKG

SEQ ID NO: 35 - protein, 11 aa, Artificial Sequence
  Synthetic
IKLGTVTTVDY

SEQ ID NO: 36 - protein, 22 aa, Artificial Sequence
  Synthetic
QSALTQPASVSGSPGQSITISC

SEQ ID NO: 37 - protein, 15 aa, Artificial Sequence
  Synthetic
WYQQHPGKAPKLMIY

SEQ ID NO: 38 - protein, 32 aa, Artificial Sequence
  Synthetic
GVSNRFSGSKSGNTASLTISGLQAEDEADYYC

SEQ ID NO: 39 - protein, 116 aa, Artificial Sequence
  Synthetic
FGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 40 - protein, 30 aa, Artificial Sequence
  Synthetic
EVQLLESGGGLVQPGGSLRLSCAASGFTFS

SEQ ID NO: 41 - protein, 14 aa, Artificial Sequence
  Synthetic
WVRQAPGKGLEWVS

SEQ ID NO: 42 - protein, 32 aa, Artificial Sequence
  Synthetic
RFTISRDNSKNTLYLQMNSLRAEDTAVYYCAR

SEQ ID NO: 43 - protein, 341 aa, Artificial Sequence
  Synthetic
WGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 214 aa, Artificial Sequence
  Synthetic
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 448 aa, Artificial Sequence
  Synthetic
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 215 aa, Artificial Sequence
  Synthetic
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 451 aa, Artificial Sequence
  Synthetic
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 216 aa, Artificial Sequence
  Synthetic
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 49 - protein, 450 aa, Artificial Sequence
  Synthetic
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 1255 aa, Homo sapiens [sequence omitted]

SEQ ID NO: 51 - protein, 11 aa, Homo sapiens
RASQDVNTAVA

SEQ ID NO: 52 - protein, 7 aa, Homo sapiens
SASFLYS

SEQ ID NO: 53 - protein, 9 aa, Homo sapiens
QQHYTTPPT

SEQ ID NO: 54 - protein, 5 aa, Homo sapiens
DTYIH

SEQ ID NO: 55 - protein, 17 aa, Homo sapiens
RIYPTNGYTRYADSVKG

SEQ ID NO: 56 - protein, 11 aa, Homo sapiens
WGGDGFYAMDY

SEQ ID NO: 57 - protein, 23 aa, Homo sapiens
DIQMTQSPSSLSASVGDRVTITC

SEQ ID NO: 58 - protein, 15 aa, Homo sapiens
WYQQKPGKAPKLLIY

SEQ ID NO: 59 - protein, 32 aa, Homo sapiens
GVPSRFSGSRSGTDFTLTISSLQPEDFATYYC

SEQ ID NO: 60 - protein, 10 aa, Homo sapiens
FGQGTKVEIK

SEQ ID NO: 61 - protein, 30 aa, Homo sapiens
EVQLVESGGGLVQPGGSLRLSCAASGFNIK

SEQ ID NO: 62 - protein, 14 aa, Homo sapiens
WVRQAPGKGLEWVA

SEQ ID NO: 63 - protein, 32 aa, Homo sapiens
RFTISADTSKNTAYLQMNSLRAEDTAVYYCSR

SEQ ID NO: 64 - protein, 11 aa, Homo sapiens
WGQGTLVTVSS

SEQ ID NO: 65 - protein, 11 aa, Homo sapiens
KASQDVSIGVA

SEQ ID NO: 66 - protein, 7 aa, Homo sapiens
SASYRYT

SEQ ID NO: 67 - protein, 9 aa, Homo sapiens
QQYYIYPYT

SEQ ID NO: 68 - protein, 5 aa, Homo sapiens
DYTMD

SEQ ID NO: 69 - protein, 17 aa, Homo sapiens
DVNPNSGGSIYNQRFKG

SEQ ID NO: 70 - protein, 10 aa, Homo sapiens
NLGPSFYFDY

SEQ ID NO: 71 - protein, 23 aa, Homo sapiens
DIQMTQSPSSLSASVGDRVTITC

SEQ ID NO: 72 - protein, 15 aa, Homo sapiens
WYQQKPGKAPKLLIY

SEQ ID NO: 73 - protein, 32 aa, Homo sapiens; identical to SEQ ID NO: 10

SEQ ID NO: 74 - protein, 10 aa, Homo sapiens
FGQGTKVEIK

SEQ ID NO: 75 - protein, 30 aa, Homo sapiens
EVQLVESGGGLVQPGGSLRLSCAASGFTFT

SEQ ID NO: 76 - protein, 14 aa, Homo sapiens
WVRQAPGKGLEWVA

SEQ ID NO: 77 - protein, 32 aa, Homo sapiens
RFTLSVDRSKNTLYLQMNSLRAEDTAVYYCAR

SEQ ID NO: 78 - protein, 11 aa, Homo sapiens
WGQGTLVTVSS

SEQ ID NO: 79 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 450 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 214 aa, Homo sapiens
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 448 aa, Homo sapiens
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 141 aa, Homo sapiens
MGPPSASPHRECIPWQGLLLTASLLNFWNPPTTAKLTIESTPFNVAEGKEVLLLVHNLPQHLFGYSWYKGERVDGNSLIVGYVIGTQQATPGAAYSGRETIYTNASLLIHNVTQNDIGFYTLQVIKSDLVNEEATGQFHVY

SEQ ID NO: 84 - protein, 11 aa, Homo sapiens
KASQDVGTSVA

SEQ ID NO: 85 - protein, 7 aa, Homo sapiens
WTSTRHT

SEQ ID NO: 86 - protein, 8 aa, Homo sapiens
QQYSLYRS

SEQ ID NO: 87 - protein, 5 aa, Homo sapiens
TYWMS

SEQ ID NO: 88 - protein, 17 aa, Homo sapiens
EIHPDSSTINYAPSLKD

SEQ ID NO: 89 - protein, 10 aa, Homo sapiens
LYFGFPWFAY

SEQ ID NO: 90 - protein, 23 aa, Homo sapiens
DIQLTQSPSSLSASVGDRVTITC

SEQ ID NO: 91 - protein, 15 aa, Homo sapiens
WYQQKPGKAPKLLIY

SEQ ID NO: 92 - protein, 32 aa, Homo sapiens
GVPSRFSGSGSGTDFTFTISSLQPEDIATYYC

SEQ ID NO: 93 - protein, 10 aa, Homo sapiens
FGQGTKVEIK

SEQ ID NO: 94 - protein, 30 aa, Homo sapiens
EVQLVESGGGVVQPGRSLRLSCSSSGFDFT

SEQ ID NO: 95 - protein, 14 aa, Homo sapiens
WVRQAPGKGLEWVA

SEQ ID NO: 96 - protein, 32 aa, Homo sapiens
RFTISRDNSKNTLFLQMDSLRPEDTGVYFCAS

SEQ ID NO: 97 - protein, 11 aa, Homo sapiens
WGQGTPVTVSS

SEQ ID NO: 98 - protein, 106 aa, Homo sapiens
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 99 - protein, 119 aa, Homo sapiens
EVQLVESGGGVVQPGRSLRLSCSSSGFDFTTYWMSWVRQAPGKGLEWVAEIHPDSSTINYAPSLKDRFTISRDNSKNTLFLQMDSLRPEDTGVYFCASLYFGFPWFAYWGQGTPVTVSS

SEQ ID NO: 100 - protein, 11 aa, Homo sapiens
KASAAVGTYVA

SEQ ID NO: 101 - protein, 7 aa, Homo sapiens
SASYRKR

SEQ ID NO: 102 - protein, 10 aa, Homo sapiens
HQYYTYPLFT

SEQ ID NO: 103 - protein, 5 aa, Homo sapiens
EFGMN

SEQ ID NO: 104 - protein, 17 aa, Homo sapiens
WINTKTGEATYVEEFKG

SEQ ID NO: 105 - protein, 12 aa, Homo sapiens
WDFAYYVEAMDY

SEQ ID NO: 106 - protein, 23 aa, Homo sapiens
DIQMTQSPSSLSASVGDRVTITC

SEQ ID NO: 107 - protein, 15 aa, Homo sapiens
WYQQKPGKAPKLLIY

SEQ ID NO: 108 - protein, 32 aa, Homo sapiens; identical to SEQ ID NO: 10

SEQ ID NO: 109 - protein, 10 aa, Homo sapiens
FGQGTKLEIK

SEQ ID NO: 110 - protein, 30 aa, Homo sapiens
QVQLVQSGAEVKKPGASVKVSCKASGYTFT

SEQ ID NO: 111 - protein, 14 aa, Homo sapiens
WVRQAPGQGLEWMG

SEQ ID NO: 112 - protein, 32 aa, Homo sapiens
RVTFTTDTSTSTAYMELRSLRSDDTAVYYCAR

SEQ ID NO: 113 - protein, 11 aa, Homo sapiens
WGQGTTVTVSS

SEQ ID NO: 114 - protein, 108 aa, Homo sapiens
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 115 - protein, 121 aa, Homo sapiens
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 116 - protein, 10 aa, Homo sapiens
SASSSVSYMH

SEQ ID NO: 117 - protein, 7 aa, Homo sapiens
STSNLAS

SEQ ID NO: 118 - protein, 9 aa, Homo sapiens
QQRSSYPLT

SEQ ID NO: 119 - protein, 5 aa, Homo sapiens
DSYMH

SEQ ID NO: 120 - protein, 17 aa, Homo sapiens
WIDPENGDTEYAPKFQG

SEQ ID NO: 121 - protein, 11 aa, Homo sapiens
GTPTGPYYFDY

SEQ ID NO: 122 - protein, 23 aa, Homo sapiens
ENVLTQSPSSMSASVGDRVNIAC

SEQ ID NO: 123 - protein, 15 aa, Homo sapiens
WFQQKPGKSPKLWIY

SEQ ID NO: 124 - protein, 32 aa, Homo sapiens
GVPSRFSGSGSGTDYSLTISSMQPEDAATYYC

SEQ ID NO: 125 - protein, 10 aa, Homo sapiens
FGGGTKLEIK

SEQ ID NO: 126 - protein, 30 aa, Homo sapiens
QVKLEQSGAEVVKPGASVKLSCKASGFNIK

SEQ ID NO: 127 - protein, 14 aa, Homo sapiens
WLRQGPGQRLEWIG

SEQ ID NO: 128 - protein, 32 aa, Homo sapiens
KATFTTDTSANTAYLGLSSLRPEDTAVYYCNE

SEQ ID NO: 129 - protein, 11 aa, Homo sapiens
WGQGTLVTVSS

SEQ ID NO: 130 - protein, 106 aa, Homo sapiens
ENVLTQSPSSMSASVGDRVNIACSASSSVSYMHWFQQKPGKSPKLWIYSTSNLASGVPSRFSGSGSGTDYSLTISSMQPEDAATYYCQQRSSYPLTFGGGTKLEIK

SEQ ID NO: 131 - protein, 120 aa, Homo sapiens
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSS

SEQ ID NO: 132 - protein, 10 aa, Homo sapiens
SASSSVPYMH

SEQ ID NO: 133 - protein, 7 aa, Homo sapiens
LTSNLAS

SEQ ID NO: 134 - protein, 9 aa, Homo sapiens
QQRSSYPLT

SEQ ID NO: 135 - protein, 5 aa, Homo sapiens
DSYMH

SEQ ID NO: 136 - protein, 17 aa, Homo sapiens
WIDPENGDTEYAPKFQG

SEQ ID NO: 137 - protein, 11 aa, Homo sapiens
GTPTGPYYFDY

SEQ ID NO: 138 - protein, 23 aa, Homo sapiens
ENVLTQSPSSMSVSVGDRVTIAC

SEQ ID NO: 139 - protein, 15 aa, Homo sapiens
WLQQKPGKSPKLLIY

SEQ ID NO: 140 - protein, 32 aa, Homo sapiens
GVPSRFSGSGSGTDYSLTISSVQPEDAATYYC

SEQ ID NO: 141 - protein, 10 aa, Homo sapiens
FGGGTKLEIK

SEQ ID NO: 142 - protein, 30 aa, Homo sapiens; identical to SEQ ID NO: 126

SEQ ID NO: 143 - protein, 14 aa, Homo sapiens
WLRQGPGQRLEWIG

SEQ ID NO: 144 - protein, 32 aa, Homo sapiens; identical to SEQ ID NO: 128

SEQ ID NO: 145 - protein, 11 aa, Homo sapiens
WGQGTLVTVSS

SEQ ID NO: 146 - protein, 106 aa, Homo sapiens
ENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 147 - protein, 120 aa, Homo sapiens; identical to SEQ ID NO: 131

SEQ ID NO: 148 - protein, 5 aa, Artificial Sequence
  Synthetic
NYGVH

SEQ ID NO: 149 - protein, 16 aa, Artificial Sequence
  Synthetic
VIWSGGNTDYNTPFTS

SEQ ID NO: 150 - protein, 11 aa, Artificial Sequence
  Synthetic
ALTYYDYEFAY

SEQ ID NO: 151 - protein, 11 aa, Artificial Sequence
  Synthetic
RASQSIGTNIH

SEQ ID NO: 152 - protein, 7 aa, Artificial Sequence
  Synthetic
YASESIS

SEQ ID NO: 153 - protein, 9 aa, Artificial Sequence
  Synthetic
QQNNNWPTT

SEQ ID NO: 154 - protein, 7 aa, Artificial Sequence
  Synthetic
SGDYYWT

SEQ ID NO: 155 - protein, 16 aa, Artificial Sequence
  Synthetic
HIYYSGNTNYNPSLKS

SEQ ID NO: 156 - protein, 9 aa, Artificial Sequence
  Synthetic
DRVTGAFDI

SEQ ID NO: 157 - protein, 11 aa, Artificial Sequence
  Synthetic
QASQDISNYLN

SEQ ID NO: 158 - protein, 7 aa, Artificial Sequence
  Synthetic
DASNLET

SEQ ID NO: 159 - protein, 9 aa, Artificial Sequence
  Synthetic
QHFDHLPLA

SEQ ID NO: 160 - protein, 9 aa, Artificial Sequence
  Synthetic
GGSISSGDY

SEQ ID NO: 161 - protein, 5 aa, Artificial Sequence
  Synthetic
YYSGS

SEQ ID NO: 162 - protein, 11 aa, Artificial Sequence
  Synthetic
VSIFGVGTFDY

SEQ ID NO: 163 - protein, 11 aa, Artificial Sequence
  Synthetic
RASQSVSSYLA

SEQ ID NO: 164 - protein, 7 aa, Artificial Sequence
  Synthetic
DASNRAT

SEQ ID NO: 165 - protein, 9 aa, Artificial Sequence
  Synthetic
HQYGSTPLT

SEQ ID NO: 166 - protein, 119 aa, Artificial Sequence
  Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 167 - protein, 107 aa, Artificial Sequence
  Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 168 - protein, 607 aa, Artificial Sequence
  synthetic
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 169 - RNA, 19 nt, Artificial Sequence
  synthetic
  misc_feature (1)..(1): n is guanosine triphosphate
ncaugcgaccucuguuuga

SEQ ID NO: 170 - RNA, 19 nt, Artificial Sequence
  synthetic
ucaaacagaggucgcaugc